(19) United States
(12) Patent Application Publication
(10) Pub. No.: US 2025/0195672 A1
(43) Pub. Date: Jun. 19, 2025

(54) DENDRIMER COMPOSITIONS FOR TARGETED DELIVERY OF THERAPEUTICS TO NEURONS

(71) Applicant: The Johns Hopkins University, Baltimore, MD (US)

(72) Inventors: Kannan Rangaramanujam, Highland, MD (US); Rishi Sharma, Baltimore, MD (US); Anjali Sharma, Baltimore, MD (US); Sujatha Kannan, Highland, MD (US); Nirnath Sah, Baltimore, MD (US); Mira Sachdeva, Baltimore, MD (US); Siva P. Kambhampati, Baltimore, MD (US)

(21) Appl. No.: 18/854,897

(22) PCT Filed: Apr. 5, 2023
(86) PCT No.: PCT/US2023/017548

§ 371 Date: Oct. 7, 2024

Related U.S. Application Data
(60) Provisional application No. 63/327,610, filed on Apr. 5, 2022.

(51) Int. Cl. A61K 47/61; A61K 9/00; A61K 45/06; A61K 49/00; A61P 25/08; C08G 83/00

(52) U.S. Cl. CPC A61K 47/61; A61K 9/0019; A61K 45/06; A61K 49/0054; A61P 25/08; C08G 83/003

(57) ABSTRACT

Glucose dendrimers synthesized using a hypercore and glucose monosaccharide-based branching units significantly enhances accumulation in neurons in the brain and in retina when administered in vivo, as compared with dendrimers without glucose monosaccharide-based branching units such as PAMAM. Compositions of glucose dendrimers conjugated with one or more therapeutic, prophylactic or diagnostic agents to prevent, treat, or diagnose a disease or disorder in a subject in need thereof, and methods of use thereof, have been developed. The compositions are particularly suited for treating and/or ameliorating diseases or disorders associated with diseased neurons in the eye or the brain. Methods of treating a human subject having or at risk of a neurological disease or disorder are provided.

### CROSS-REFERENCE TO RELATED APPLICATIONS

This application claims the benefit of and priority to U.S. Provisional Application No. 63/327,610 filed on Apr. 5, 2022, the contents of which is incorporated herein in its entirety.

### FIELD OF THE INVENTION

The invention is generally in the field of drug delivery and, in particular, dendrimer compositions and methods for delivering drugs selectively to neurons.

### BACKGROUND OF THE INVENTION

Preserving neurons in the context of a brain injury is a major challenge and is a primary goal in the treatment of many neurological disorders. However, targeting injured neurons specifically, whether through local administration into the brain or from systemic administration, has been a major challenge especially considering their relatively low endocytosing rates compared to immune cells (Krol, S., Journal of Controlled Release. 164(2), 145-55(2012)).

For example, brain injury can result in neuronal hyperexcitability that can contribute to several neurologic conditions like epilepsy, chronic pain and Parkinson's disease (Anastacio, et al, Translational Psychiatry 12:1-14 (2022); G. Carola et al., Parkinson's Disease 7:1-14 (2021)). Therapeutic targeting of select hyperactive neurons, not only, can rescue neurons from excitotoxic death, but can also limit disease propagation. However, selective targeting of neurons remains elusive, primarily because of the blood-brain barrier (BBB) and the spectrum of neuronal properties and functions exhibited in different brain regions impacted in diverse neurologic conditions. Systemically administered drugs must cross the blood brain barrier, diffuse freely in the brain tissue and be selectively taken up by the target cells. This is a major challenge, with many ligand, antibody, viral vector-based approaches having been examined, with mixed results (S. Krol, J Control Release 164: 145-155 (2012); E. S. Smith, et al., Advanced drug delivery reviews 148: 181-203 (2019); J. Garcia-Chica et al., Nanomedicine (Lond) 15: 1617-1636 (2020); A. P. Spencer et al., Pharmaceutics:12 (2020)). Even if therapeutic drugs manage to cross the BBB, targeting neurons selectively remains a significant challenge and must be carefully considered as it can result in undesirable side effects. Since neurons function in a complex and diverse fashion in different brain regions, with regionally different overexpression of receptors, targeting subtypes of neurons in different regions of brain using a ligand-based strategy may be impractical for broad applications (J. Garcia-Chica et al., Nanomedicine (Lond) 15: 1617-1636 (2020); F. Zhang, Y. et al., J. Control Release 240: 212-226 (2016)). Among myriads of differences, one common characteristics in most brain injury is higher metabolic activity and demand for glucose in neurons, at least in the acute phase of the injury or neuro-disease. Glucose is the primary metabolic source for brain (B. Siesjö, Journal of neural transmission, 17-22 (1978)) and is transported across the BBB and made available to neurons and glia via specific glucose transporters (L. Pellerin, Proc. Nat. Acad. Sci. 91:10625-10629 (1994); L. K. Bak et al., J. Neurochem.109: 87-93 (2009); Diaz-Garcia et al., Cell metabolism 26: 361-374. e364 (2017); Tredern et al., Cell Reports 36: 109620 (2021)). In the presence of an excitotoxic injury, such as that seen with epilepsy, there is increased glucose transport into the affected neurons due to increased metabolic demand. Glut3 and SGLT transporters expressed by neurons can transport glucose from the interstitium and generate ATP through glycolysis, pentose phosphate pathway and oxidative metabolism depending on the extent of neuronal stimulation (C. M. Díaz-García et al., Cell metabolism 26: 361-374. e364 (2017); I. Lundgaard et al., Nat Commun 6, 6807 (2015); Pellerin et al., Glia, 1251-1262 (2007); Herrero-Mendez et al., Nature cell biology 11:747-752 (2009); J. Jurcovicova, Endocr Regul 48:35-48 (2014)).

At present, the delivery of therapeutics including small molecule drugs and large molecular weight biologics to the nucleus of neurons is a challenge. New systems for targeted delivery to injured neurons and selective delivery of drugs to the site of pathology for treating neurological disorders, such as Alzheimer's, Parkinson's, cerebral palsy, autism, multiple sclerosis, spinal muscular atrophy, traumatic brain injury, glaucoma, and other retina disorders, are needed.

Therefore, it is an object of the present invention to provide compositions that selectively deliver therapeutic, prophylactic, or diagnostic agents to target cells within the site of pathology in the eye, the brain, or the CNS including neurons, and methods of making and using thereof.

It is also an object of the invention to provide compositions for the treatment or prevention of one or more symptoms of neurological disorders and retinal disorders through direct targeting of the diseased cells.

### SUMMARY OF THE INVENTION

Dendrimer compositions, referred to as “glucose dendrimers, that can selectively accumulate inside neurons, particularly in the nucleus of injured and/or hyperactive neurons”, have been developed. These dendrimers can accumulate at a high level inside activated microglia. In contrast to hydroxyl dendrimers which primarily accumulate in microglia, these dendrimers primarily go to neurons.

Glucose dendrimers include (a) a central core, (b) one or more branching units, wherein the branching units are monosaccharide glucose-based branching units, optionally with a linker conjugated thereto; and optionally (c) one or more therapeutic, prophylactic and/or diagnostic agents. Generally, the one or more branching units are conjugated to the central core, and the surface groups of the dendrimer are monosaccharide glucose molecules. In some embodiments, the central core is dipentaerythritol, or a hexa-propargylated derivative thereof. In some embodiments, the branching unit is conjugated to the central core via a linker such as a hydrocarbon or an oligoethylene glycol chain. In a preferred embodiment, the branching units are β-D-Glucopyranoside tetraethylene glycol azide having the following structure,

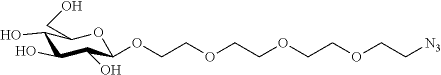

or peracetylated derivatives thereof.

In some embodiments, the glucose dendrimer is a generation 1, generation 2, generation 3, generation 4, generation 5, or generation 6 dendrimer. In one embodiment, the dendrimer is a generation 1 dendrimer having the following structure:

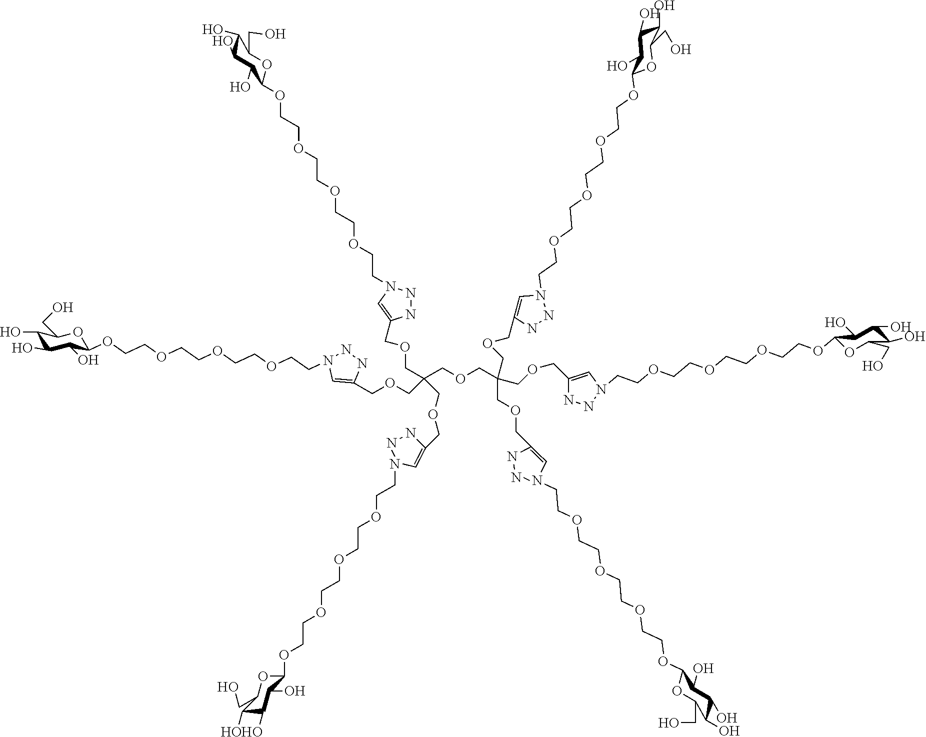

In a preferred embodiment, the dendrimer is a generation 2 dendrimer having the following structure:

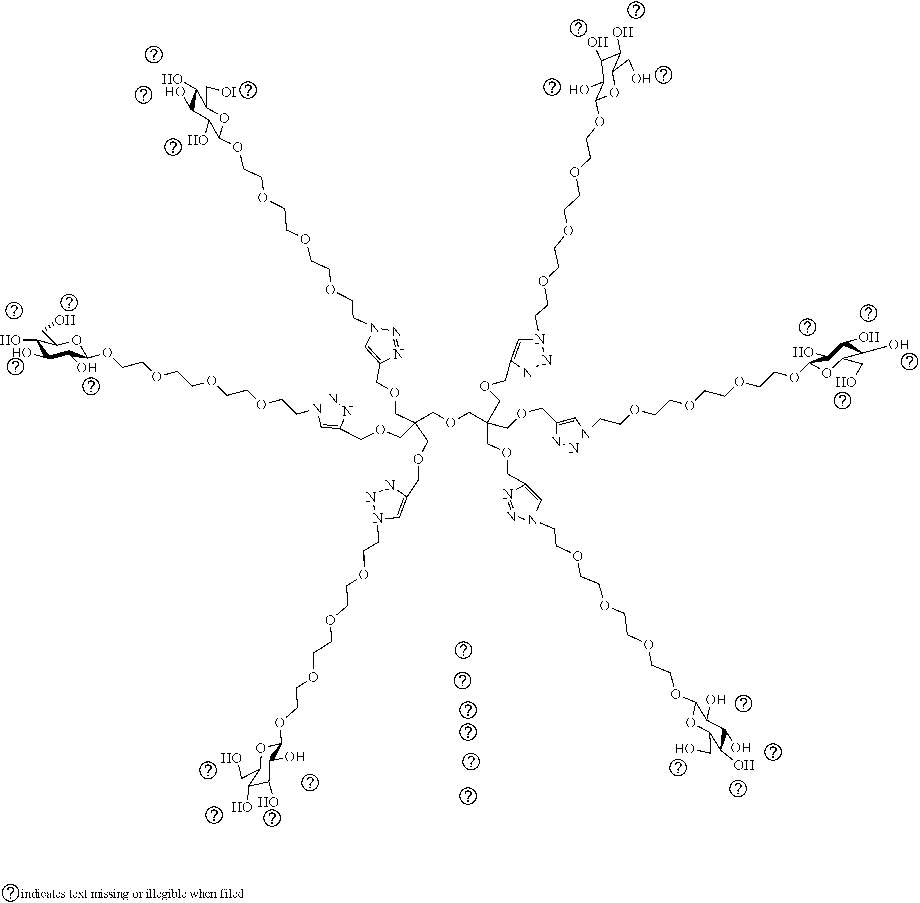

In some embodiments, the one or more therapeutic agents, prophylactic agents, and/or diagnostic agents are encapsulated, associated, and/or conjugated in the dendrimer, at a concentration of between about 0.01% to about 30%, preferably about 1% to about 20%, more preferably about 5% to about 20% by weight. In some embodiments, the dendrimer is conjugated to a small molecule, an antibody or antigen-binding fragment thereof, a nucleic acid, or a polypeptide. In some embodiments, the therapeutic agents conjugated to the dendrimer are anti-inflammatory agents, antioxidant agents, or immune-modulating agents. In other embodiments, the dendrimers are conjugated to one or more diagnostic agents such as fluorescent dyes, near infra-red dyes, SPECT imaging agents, PET imaging agents, and radioisotopes.

In some embodiments, the dendrimer and the therapeutic, prophylactic, or diagnostic agent(s) are conjugated via one or more linkers or coupling agents such as one or more hydrocarbon or oligoethylene glycol chains. Exemplary linkages are disulfide, ester, ether, thioester, and amide linkages.

Pharmaceutical formulations including the glucose dendrimers typically include the dendrimer composition and one or more pharmaceutically acceptable carriers or excipients. In some embodiments, the formulation is formulated for systemic administration. In some embodiments, the formulation is formulated for enteral or parenteral administration such as intramuscular, intraperitoneal, intravenous, or subcutaneous injection administration.

Methods for treating or preventing one or more diseases, conditions, and/or injuries of the eye, the brain and/or the nervous system (CNS) by administering to a subject in need thereof the pharmaceutical formulation of the glucose dendrimers are also provided. Typically, the one or more diseases, conditions, and/or injuries of the eye, the brain and/or the nervous system are diseases, conditions, and injuries associated with neurons and/or activated microglia. In some embodiments, the one or more diseases, conditions, and/or injuries of the eye are eye diseases associated with retinal ganglion cells, for example, glaucoma, diabetic retinopathy, acute retinal ischenia, traumatic optic nerve injury, optic nerve atrophy, and Leber's hereditary optic neuropathy, wherein the one or more therapeutic agents encapsulated, associated, and/or conjugated in the dendrimer are ROCK inhibitors, α-2 adrenergic receptor agonists, or caspase inhibitors. In some embodiments, the one or more diseases, conditions, and/or injuries of the brain and/or the nervous system are neurological and/or neurodegenerative diseases such as traumatic brain injury, demyelinating diseases, epilepsy, neuralgia, Alzheimer's disease, Parkinson's disease, Huntington's disease, cerebral palsy, autism, multiple sclerosis, spinal muscular atrophy, neuronal ceroid lipofuscinoses, and neuronopathic Goucher disease. In these cases, exemplary therapeutic agents encapsulated, associated, and/or conjugated in the dendrimer include calpain inhibitors, GPR52 antagonists, NMDA antagonists, mTOR inhibitors, LLRK2 inhibitors, nuclear factor erythroid 2 related factor 2 activators, and SMN-2 promotors. In other embodiments, the one or more diseases, conditions, and/or injuries of the brain and/or the nervous system are neurological diseases associated with motor neurons such as amyotrophic lateral sclerosis, primary lateral sclerosis, progressive bulbar palsy, pseudo bulbar palsy, progressive muscular atrophy, spinal muscular atrophy, Kennedy's disease. In one embodiment, the neurological disease is spinal muscular atrophy, and optionally the therapeutic agents encapsulated, associated, and/or conjugated in the dendrimer are HDAC inhibitors or antisense oligonucleotides such as nusinersen. The dendrimer formulation can be administered orally, intravenously, intraperitoneally, or intravitreally. In preferred embodiments, the amount of therapeutic, prophylactic or diagnostic agent effective to treat or prevent the one or more symptoms is less than the amount of the same therapeutic, prophylactic or diagnostic agent administered in the absence of the glucose dendrimers, or administered as a formulation in combination with dendrimers in the absence of surface glucose molecules.

Methods for labeling one or more neurons and/or activated microglia associated with one or more diseases, conditions, and/or injuries of the eye, the brain and/or the nervous system (CNS) are also provided. Methods include administering to the subject an effective amount of the pharmaceutical formulation of the glucose dendrimers to label one or more cells associated with the one or more diseases, conditions, and/or injuries of the eye, the brain and/or the nervous system (CNS). In some embodiments, the labeling is used to diagnose or identify the one or more diseases, conditions, and/or injuries of the eye, the brain and/or the nervous system (CNS) in the subject. In other embodiments, the labeling is used to monitor or guide therapy and/or surgery. The dendrimer formulation is administered orally, intravenously, subchondroidally, or intravitreally.

Methods of delivering one or more therapeutic, prophylactic, or diagnostic agents to one or more neurons in a subject in need thereof are also provided. Methods include administering to the subject an effective amount of the pharmaceutical formulation of the glucose dendrimers. In some embodiments, the dendrimers deliver to one or more neurons including cerebral cortex neurons, motor neurons, dopaminergic neurons, hypothalamus neurons, thalamus neurons, brain stem neurons, raphe nucleus neurons, Purkinje neurons, retinal ganglion cells, and other neurons in of the central nervous system. In preferred embodiments, the amount of dendrimer administered results in the one or more therapeutic, prophylactic or diagnostic agents accumulating within the one or more neurons to at least 5-fold, 10-fold, 20-fold, 30-fold, 40-fold, or 50-fold more than the amount of the same therapeutic, prophylactic or diagnostic agent administered in the absence of the dendrimers, or administered as a formulation in combination with dendrimers in the absence of surface glucose molecules.

### BRIEF DESCRIPTION OF THE DRAWINGS

FIGS. 1A-1B are schematics showing molecular structures in a stepwise synthetic route for producing an exemplary glucose dendrimer (FIG. 1A), and a stepwise synthetic route for conjugating an exemplary agent, Cy5, to the dendrimer (FIG. 1B).

FIGS. 2A-2E are schematics showing an exemplary synthetic route for glucose dendrimer-drug conjugates using copper catalyzed alkyne-azide click (CuAAC) chemistry (FIG. 2A), and a stepwise synthetic route for conjugating exemplary therapeutic, prophylactic or diagnostic agents, loperamide (FIG. 2B), rapamycin (FIG. 2C), valproic acid (FIGS. 2D and 2E) to the dendrimer.

FIGS. 3A-3B are schematics showing an exemplary synthetic route for glucose dendrimer-drug conjugates using a combination of click and esterification/amidation reactions (FIG. 3A), and a stepwise synthetic route for conjugating an exemplary active agent, N-acetyl cysteine, to the dendrimer (FIG. 3B).

FIG. 4 is a scheme showing an exemplary synthetic route for glucose dendrimer-drug conjugates using copper-free biorthogonal click chemistry.

FIG. 5 is a bar graph showing mean dendrimer fluorescence intensity (Cy5 channel) from contralateral CA1 neurons in mice with intracranial injection of PAMAM-OH-Cy5 or GD2-Cy5 (n=32 neurons from 2 mice). *p<0.001.

FIG. 6A is a scheme showing Thy1-YFP mouse brain removal and transfer to culture media for incubation with indicated agents prior to formalin fix and imaging analysis. FIG. 6B is a bar graph showing mean dendrimer fluorescence intensity in Mg₂+ free artificial cerebrospinal fluid (ACSF) in control conditions (n=187 neurons), or in samples pre-treated with cytochalasin B (n=104 neurons) or glutor (n=128 neurons) or phlorizin (n=80 neurons), as well as in medium with high Mg₂+ and N-Methyl-D-glucamine (NMDG) (n=69 neurons), *p<0.001.

FIG. 7 is a scheme showing an exemplary synthetic route to make glucose dendrimer (GD) and fluorescently labeled glucose dendrimer (GD-Cy5).

FIGS. 8A and 8B demonstrate that GD2 targets CA1 neurons in mouse model of temporal lobe epilepsy. FIG. 8A is a schemate depicting experimental timeline. PAMAM-OH or GD2 conjugated to Cy5 was intra-cranially administered in the right hemisphere. After overnight recovery, pilocarpine was injected to induce seizures and 30 min upon observing active behavioral seizures (Racine scale 3 or above), mice were sacrificed, perfused and brain collected for immunohistochemistry. FIG. 8B is a bar graph showing the mean dendrimer fluorescence intensity (Cy5 channel) from contralateral CA1 neurons show approximately 100 fold higher uptake of GD2 (n=56 neurons from 2 mice) than PAMAM-OH (n=32 neurons from 2 mice). *p<0.001.

FIGS. 9A and 9B demonstrate that neuronal activity and GLUT transporters mediate GD2 uptake. FIG. 9A is scheme of the experiment in which 300 μm cortical brain sections were pre-treated for 30 minutes with control ACSF (Mg2+ free, increases neuronal firing) or ACSF containing Mg2+/NMDG (suppressed neuronal activity) or control ACSF with either cytochalasin B (5 μM) or glutor (I₁M). After pre-treatment, brain sections were incubated with GD2-Cy5 (10 μg/ml) for 30 minutes followed by 10% formalin fixation and confocal imaging. Mean fluorescence intensities for GD-Cy5 were evaluated from YFP-expressing cortical neurons FIG. 9B is a bar graph showing that GD2-Cy5 uptake was significantly decreased when neuronal activity was suppressed (n=69 neurons), pre-treated with cytochalasin B (n=104 neurons) or glutor (n=128 neurons) compared to control incubation condition (n=187 neurons). *p<0.001

FIGS. 10A and 10B show synthetic route to clickable VPA-azide (FIG. 10A); glucose-dendrimer-VPA conjugate (GD2-VPA) (FIG. 10B). At pH 7.4, GD2-VPA conjugate is stable with no indication of VPA release up to 24 h. Under intracellular conditions, GD2-VPA conjugates show fast release of VPA with ˜15% release in 1-2 hours and 25% in 24 hours.

FIGS. 11A and 11B show that intranasal GD2-VPA decreases seizure severity induced by pilocarpine. FIG. 11A is a scheme timeline of the experiment. FIG. 11B is a bar graph showing that mice treated with GD2-VPA had better mobility and activity than the saline treated animals after 1 hour of pilocarpine administration.

FIGS. 12A-12I show uptake of GD2-Cy5 dendrimer by select neurons (Syngap mouse seizure model). FIG. 12A is an experimental timeline. FIGS. 12B and 12C are bar graphs of the scores for seizure duration and latency to high grade seizures on day 1. FIGS. 12D-12F are bar graphs showing the seizure duration scores for low grade seizures (FIG. 12D), medium grade seizures (FIG. 12E), and high grade seizures (FIG. 12F). FIGS. 12G-12I are bar graphs showing the day 2 seizure duration and latency to high grade seizures for low grade seizures (FIG. 12G), medium grade seizures (FIG. 12H), and high grade seizures (FIG. 12I).

### DETAILED DESCRIPTION OF THE INVENTION

### I. Definitions

The terms “active agent” or “biologically active agent” are used interchangeably to refer to a chemical or biological compound that induces a desired pharmacological and/or physiological effect, which may be prophylactic, therapeutic, or diagnostic. These may be a nucleic acid, a nucleic acid analog, a small molecule having a molecular weight less than 2 kD, more typically less than 1 kD, a peptidomimetic, a protein or peptide, carbohydrate or sugar, lipid, or a combination thereof. The terms also encompass pharmaceutically acceptable, pharmacologically active derivatives of agents, including, but not limited to, salts, esters, amides, prodrugs, active metabolites, and analogs. The term “therapeutic agent” refers to an agent that can be administered to treat one or more symptoms of a disease or disorder. The term “diagnostic agent” generally refers to an agent that can be administered to reveal, pinpoint, and define the localization of a pathological process. The diagnostic agents can label target cells that allow subsequent detection or imaging of these labeled target cells. In some embodiments, diagnostic agents can, via dendrimer, selectively target neurons, particularly neurons within the site of pathology in the eye, the brain, or the CNS. The term “prophylactic agent” generally refers to an agent that can be administered to prevent disease or to prevent certain conditions.

The term “therapeutically effective amount” refers to an amount of the therapeutic agent that, when incorporated into and/or onto dendrimers, produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation. In some embodiments, the term “effective amount” refers to an amount of a therapeutic agent or prophylactic agent to reduce or diminish the symptoms of one or more ocular or neurological diseases.

The terms “inhibit” or “reduce” in the context of inhibition, mean to reduce, or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%. For example, dendrimer compositions including one or more inhibitors may inhibit or reduce the activity and/or quantity of diseased neurons by about 10%, 20%, 30%, 40%, 50%, 75%, 85%, 90%, 95%, or 99% from the activity and/or quantity of the same cells in equivalent tissues of subjects that did not receive, or were not treated with the dendrimer compositions. In some embodiments, the inhibition and reduction are compared at levels of mRNAs, proteins, cells, tissues, and organs. For example, an inhibition and reduction in the rate of neural loss, in the rate of decrease of brain weight, or in the rate of decrease of hippocampal volume, as compared to an untreated control subject.

The term “treating” or “preventing” mean to ameliorate, reduce or otherwise stop a disease, disorder or condition from occurring or progressing in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. For example, an individual is successfully “treated” if one or more symptoms associated with Alzheimer's disease are mitigated or eliminated, including, but are not limited to, reducing the rate of neuronal loss, decreasing symptoms resulting from the disease, increasing the quality of life of those suffering from the disease, decreasing the dose of other medications required to treat the disease, delaying the progression of the disease, and/or prolonging survival of individuals.

The phrase “pharmaceutically acceptable” or “biocompatible” refers to compositions, polymers, and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions, or vehicles, such as a liquid or solid filler, diluent, solvent, or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “biodegradable” generally refers to a material that will degrade or erode under physiologic conditions to smaller units or chemical species that are capable of being metabolized, eliminated, or excreted in vivo. The degradation time is a function of composition and morphology.

The term “dendrimer” includes, but is not limited to, a molecular architecture with an interior core, interior layers, or “generations” of repeating units regularly attached to this initiator core, and an exterior surface of terminal groups attached to the outermost generation.

The term “functionalize” means to modify a compound or molecule in a manner that results in the attachment of a functional group or moiety. For example, a molecule may be functionalized by the introduction of a molecule that makes the molecule a strong nucleophile or strong electrophile.

The term “targeting moiety” refers to a moiety that localizes to or away from a specific location. The moiety may be, for example, a protein, nucleic acid, nucleic acid analog, carbohydrate, or small molecule. The entity may be, for example, a therapeutic compound such as a small molecule, or a diagnostic entity such as a detectable label. The location may be a tissue, a particular cell type, or a subcellular compartment. In one embodiment, the targeting moiety directs the localization of an agent. In preferred embodiment, the dendrimer composition can selectively target neurons, particularly injured/hyperactive neurons, in the absence of an additional targeting moiety.

The term “prolonged residence time” refers to an increase in the time required for an agent to be cleared from a patient's body, or organ or tissue of that patient. In certain embodiments, “prolonged residence time” refers to an agent that is cleared with a half-life that is 10%, 20%, 50% or 75% longer than a standard of comparison such as a comparable agent without conjugation to a delivery vehicle such as a dendrimer. In certain embodiments, “prolonged residence time” refers to an agent that is cleared with a half-life of 2, 5, 10, 20, 50, 100, 200, 500, 1000, 2000, 5000, or 10000 times longer than a standard of comparison such as a comparable agent without a dendrimer that specifically target specific cell types.

The terms “incorporated” and “encapsulated” refer to incorporating, formulating, or otherwise including an agent into and/or onto a composition that allows for release, such as sustained release, of such agent in the desired application. The agent or other material can be incorporated into a dendrimer, by binding to one or more surface functional groups of such dendrimer (by covalent, ionic, or other binding interaction), by physical admixture, by enveloping the agent within the dendritic structure, and/or by encapsulating the agent inside the dendritic structure.

### II. Compositions

Dendrimers suitable for delivering one or more agents to neurons, preferably those within the site of pathology in the eye, the brain, or the CNS, have been developed. These dendrimers are particularly suited for delivering one or more agents to prevent, treat or diagnose one or more ocular diseases, one or more neurological and neurodegenerative diseases, especially dementia, other disorders associated with neuroinflammation.

### A. Dendrimers

Dendrimers are three-dimensional, hyperbranched, monodispersed, globular and polyvalent macromolecules including surface end groups (Tomalia, D. A., et al., Biochemical Society Transactions, 35, 61 (2007); and Sharma, A., et al., ACS Macro Letters, 3, 1079 (2014)). Due to their unique structural and physical features, dendrimers have shown unprecedented potential as nano-carriers for various biomedical applications including targeted drug/gene delivery, imaging and diagnosis (Sharma, A., et al., RSC Advances, 4, 19242 (2014); Caminade, A.-M., et al., Journal of Materials Chemistry B, 2, 4055 (2014); Esfand, R., et al., Drug Discovery Today, 6, 427 (2001); and Kannan, R. M., et al., Journal of Internal Medicine, 276, 579 (2014)).

Dendrimers are useful for various biomedical applications including drug/gene delivery, targeting, imaging and diagnosis (Soliman, G M et al., Chem. Commun. 2011, 47, 9572; and Tomalia, D A et al., Biochem. Soc. Trans. 2007, 35, 61). Among several different types of dendrimers, polyamidoamine (PAMAM) dendrimers have been widely explored for drug delivery applications due to their commercial availability, aqueous solubility and biocompatibility (Tomalia, D A et al., Polym J 1985, 17, 117). The small size and the presence of easily tunable multiple surface groups make these nanoparticles excellent carriers for the transport of drugs to CNS. Earlier studies show that non-cytotoxic, hydroxyl terminated generation 4 PAMAM dendrimers (˜4 nm size, without any targeting ligand) can cross the impaired BBB and target activated microglia at the site of injury in the brain several fold more than the healthy control (Lesniak, W G et al., Mol Pharm 2013, 10). These dendrimers are non-toxic even at intravenous doses greater than 500 mg/kg, and are cleared intact through the kidneys. These findings were validated in various small and large animal models (Kannan, S et al., Sci. Transl. Med. 2012, 4, 130ra46; Kambhampati, S P et al., Invest Ophthalmol Vis Sci 2015, 56; Nance, E et al., J. Control. Release 2015, 214, 112; Mishra, M K et al., ACS Nano 2014, 8, 2134; and Nanomedicine 2010, 5, 1317). The selective uptake and localization of these neutral dendrimers in activated microglia might attribute to their ability to cross the impaired BBB and diffuse rapidly in the brain parenchyma followed by the uptake by constantly phagocytic activated glial cells.

Dendrimer surface groups can have a significant impact on their biodistribution (Nance, E., et al., Biomaterials, 101, 96 (2016)). More specifically, hydroxyl terminating generation 4 PAMAM dendrimers (˜4 nm size) without any targeting ligand have been shown to cross the impaired BBB upon systemic administration in a rabbit model of cerebral palsy (CP) significantly more (>20 fold) as compared to healthy controls, and selectively target activated microglia and astrocytes (Lesniak, W. G., et al., Mol Pharm, 10 (2013); Kannan, S., et al., Science Translational Medicine, 4, 130ra46 (2012); Iezzi, R., et al., Biomaterials, 33, 979 (2012); Mishra, M. K., et al., ACS Nano, 8, 2134 (2014); Kambhampati, S. P., et al., European Journal of Pharmaceutics and Biopharmaceutics, 95, Part B, 239 (2015); Zhang, F., et al., Journal of Controlled Release, 249, 173 (2017); Guo, Y., et al., PLOS ONE, 11, e0154437 (2016); and Inapagolla, R., et al., International Journal of Pharmaceutics, 399, 140 (2010)).

The term “dendrimer” includes, but is not limited to, a molecular architecture with an interior core (“G0”) and layers (or “generations”) of repeating units which are attached to and extend from this interior core, each layer having one or more branching points, and an exterior surface of terminal groups attached to the outermost generation. In some embodiments, dendrimers have regular dendrimeric or “starburst” molecular structures. The dendrimers can have carboxylic, amine, or hydroxyl terminations, and can be of any generation including, but not limited to, generation 1 (“G1”) dendrimers (“D1”), generation 2 (“G2”) dendrimers (“D2”), generation 3 (“G3”) dendrimers (“D3”), generation 4 (“G4”) dendrimers (“D4”), generation 5 (“G5”) dendrimers (“D5”), generation 6 (“G6”) dendrimers (“D6”), generation 7 (“G7”) dendrimers (“D7”), generation 8 (“G8”) dendrimers (“D8”), generation 9 (“G9”) dendrimers (“D9”), or generation 10 (“G10”) dendrimers (“D10”).

Generally, dendrimers have a diameter between about 1 nm and about 50 nm, more preferably between about 1 nm and about 20 nm, between about 1 nm and about 10 nm, or between about 1 nm and about 5 nm. In some embodiments, the diameter is between about 1 nm and about 2 nm, inclusive; between about 2 nm and about 3 nm, inclusive; between about 3 nm and about 5 nm, inclusive; or between about 4 nm and about 5 nm, inclusive. In preferred embodiments, the dendrimers have a diameter effective to penetrate brain tissue and to retain in target cells for a prolonged period of time.

In some embodiments, dendrimers have a molecular weight between about 500 Daltons and about 100,000 Daltons inclusive, preferably between about 500 Daltons and about 50,000 Daltons inclusive, most preferably between about 1,000 Daltons and about 20,000 Daltons inclusive.

In some embodiments, the dendrimers have a hypercore (e.g., dipentaerythritol) and one or more monosaccharide branching units. In some embodiments, the monosaccharide branching units are conjugated to the core or the prior layer of monomers via linkers such as polyethylene glycol chains. In preferred embodiments, the hypercore is dipentaerythritol and the monosaccharide branching unit is glucose-based branching unit such as shown in Structures I-III.

In some embodiments, the dendrimer specifically targets a particular tissue region and/or cell types following administration into the body. In preferred embodiments, the dendrimer specifically targets a particular tissue region and/or cell type without a targeting moiety. In one embodiment, the glucose dendrimer selectively targets or enriched inside neurons, specifically the nucleus of neurons. In a preferred embodiment, the glucose dendrimer selectively targets or enriched inside injured, diseased, and/or hyperactive neurons.

### 1. Central Core

A multifunctional core moiety allows stepwise addition of branching units (i.e., generations) around the core.

Exemplary chemical structures suitable as core moieties include dipentaerythritol, pentaerythritol, 2-(aminomethyl)-2-(hydroxymethyl) propane-1,3-diol, 2-ethyl-2-(hydroxymethyl) propane-1,3-diol, 3,3′,3′″-silanetetrayltetrakis (propane-1-thiol), 3,3-divinylpenta-1,4-diene, 3,3′,3″-nitrilotripropionic acid, 3,3′,3″-nitrilotris(N-(2-aminoethyl)propanamide), 3,3′,3″,3′″-(ethane-1,2-diylbis(azanetriyl)) tetrapropanamide, 3-(carboxymethyl)-3-hydroxypentanedioic acid, 2,2′-((2,2-bis((2-hydroxyethoxy)methyl) propane-1,3-diyl)bis(oxy))bis(ethan-1-ol), tetrakis(3-(trichlorosilyl) propyl)silane, 1-Thioglycerol, 2,2,4,4,6,6-hexachloro-1,3,5,215,415,615-triazatriphosphinine, 3-(hydroxymethyl)-5,5-dimethylhexane-2,4-diol, 4,4′,4″-(ethane-1,1,1-triyl)triphenol, 2,4,6-trichloro-1,3,5-triazine, 5-(hydroxymethyl) benzene-1,2,3-triol, 5-(hydroxymethyl)benzene-1,3-diol, 1,3,5-tris(dimethyl(vinyl)silyl)benzene, Carbosiloxane core, nitrilotrimethanol, ethylene diamine, propane-1,3-diamine, butane-1,4-diamine, 2,2′,2″-nitrilotris(ethan-1-ol), alpha cyclodextrin, beta cyclodextrin, gamma cyclodextrin, Cucurbituril, benzene-1,2,3,4,5,6-hexathiol, monosaccharide, disaccharides, trisaccharides, oligosaccharides, or azide-, alkyne-modified moieties thereof. In some embodiments, the core moiety is chitosan. Thus, azide-modified chitosan, or alkyne-modified chitosan are suitable for conjugating to branching units using click chemistry.

In preferred embodiments, the core moiety is dipentaerythritol.

### 2. Branching Units

Exemplary chemical structures suitable as branching units include monosaccharide. In some embodiments, the monosaccharide branching units are conjugated to the core or the prior layer of monomers via linkers such as polyethylene glycol chains. In preferred embodiments, the monosaccharide branching units are glucose-based branching units. Exemplary glucose-based branching units are shown in Structures I-III.

These are spacer molecules, so can also be alkyl (CH₂)ₙ—hydrocarbon-like units.

The branching units are the PEG or alkyl chain linkers between different dendrimer generations, for example, the glucose layers are connected via PEG linkers and triazole rings.

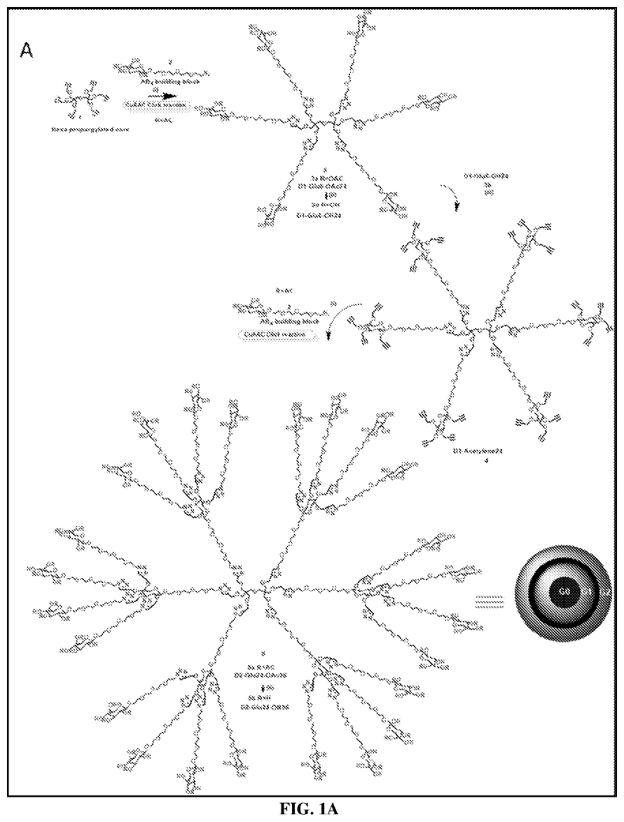

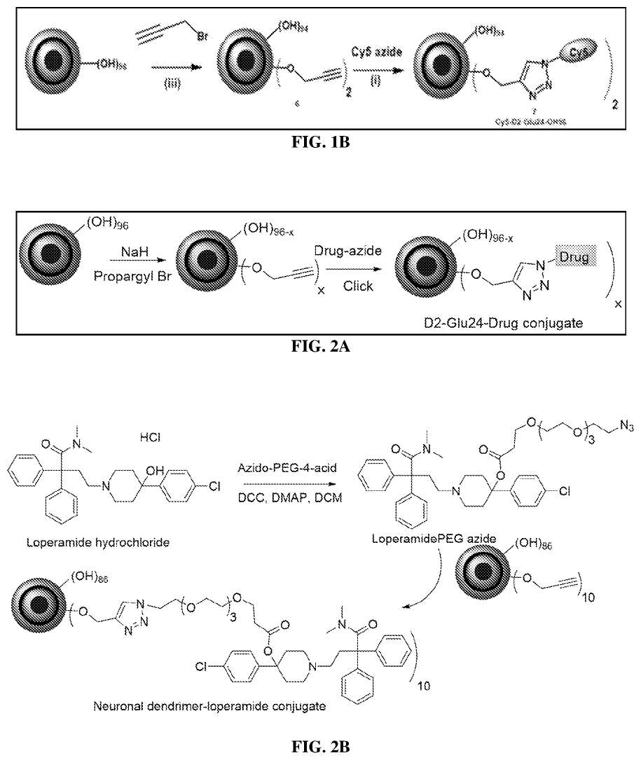

In preferred embodiments, the branching units are the same for each generation of dendrimers generated from the core. Therefore, in one embodiment, the branching units are glucose-based branching units for generating generation 1 dendrimers as shown in Structures IV-VI, and for generating generation 2 dendrimers as shown in FIGS. 1A and 1B.

In some embodiments, the branching units are hyper-monomers i.e., ABₙ building blocks. Exemplary hyper-monomers include AB₄, AB₅, AB₆, AB₇, AB₈ building blocks. Hyper-monomer strategy drastically increases the number of available end groups. An exemplary AB₄ hypermonomer is peracetylated β-D-Glucopyranoside tetraethylene glycol azide as shown in Structure II.

### 3. Surface Groups

Surface groups or terminal functional groups are preferably hydroxyl groups of the terminal glucose of the branching units. In some embodiments, desired surface groups can be modified or added via one of the conjugation methods for the core and branching unit. Exemplary surface groups include hydroxyl end groups, amine end groups, carboxylic acid end groups, and thiol ends.

In some embodiments, the dendrimers are able to specifically target a particular tissue region and/or cell type, preferably the cells and tissues of the central nervous system (CNS) and the eye. In some embodiments, the dendrimers specifically target neurons of the CNS and the eye. Unmodified PAMAN dendrimers with hydroxyl end groups do not enrich in the neurons of brain and/or retinal ganglion cells (RGCs) in the eye as much as these glucose dendrimers. The glucose dendrimers with terminal glucose monosaccharide and a high density of hydroxyl functional groups effectively target the neurons in a generation dependent manner. Examples demonstrate efficacy with generation 2 (G2), and G3 and G4 should be efficacious. G5 and above are more difficult to use.

In preferred embodiments, the dendrimers include an effective number of terminal glucose and/or hydroxyl groups for targeting to one or more neurons of the CNS, or the eye. The hydroxyl groups on the dendrimer surface are part of glucose molecules. There are no extra hydroxyls in addition to the glucose molecules on the surface. The number of sugar molecules on the surface is determined by the generation number. All generations are expected to target neurons.

In some embodiments, dendrimers are made of glucose and oligoethylene glycol building blocks. Exemplary glucose dendrimers are shown in the Examples, for example, generation 1 dendrimers as shown in Structures IV-VI, and generation 2 dendrimers as shown in FIGS. 1A and 1B. Some exemplary glucose dendrimers include a generation 1 glucose dendrimer having 24 hydroxyl (—OH) end groups, a generation 2 glucose dendrimer having 96 hydroxyl (—OH) end groups, a generation 3 glucose dendrimer having 396 hydroxyl (—OH) end groups, and generation 4 glucose dendrimer having 1584 hydroxyl (—OH) end groups. In a preferred embodiment, the glucose dendrimer is a generation 2 glucose based dendrimer that has 24 glucose molecules at the periphery and 6 embedded glucose molecules in the backbone held together by PEG segments.

### B. Linking Chemistry and Spacers

Dendrimer conjugates can be formed of therapeutic, prophylactic and/or diagnostic agents or compounds conjugated or attached to a glucose dendrimer. Optionally, the therapeutic, prophylactic or diagnostic agents are conjugated to the dendrimers via one or more spacers/linkers via different linkages such as disulfide, ester, carbonate, carbamate, thioester, hydrazine, hydrazides, and amide linkages. The one or more spacers/linkers between a dendrimer and an therapeutic, prophylactic or diagnostic agent can be designed to provide a releasable or non-releasable form of the dendrimer-active complexes in vivo. In some embodiments, the attachment occurs via an appropriate spacer that provides an ester bond between the agent and the dendrimer. In some embodiments, one or more spacers/linkers between a dendrimer and an agent are added to achieve desired and effective release kinetics in vivo. These may be cleavable (Ester, S-S) or non-cleavable (amide, ether), The linking chemistry can be click chemistry, acid-amine coupling, Steglich esterification etc.

### 1. Coupling Agents

In some embodiments, the therapeutic, prophylactic or diagnostic agents are attached to the dendrimer via a linking moiety that is designed to be cleaved in vivo. The linking moiety can be designed to be cleaved hydrolytically, enzymatically, or by a combination thereof, to provide for the sustained release of the agents in vivo. Both the composition of the linking moiety and its point of attachment to the agent are selected so that cleavage of the linking moiety releases either an therapeutic, prophylactic or diagnostic agent or a prodrug thereof. The composition of the linking moiety can also be selected in view of the desired release rate of the agents.

In some embodiments, the attachment occurs via one or more of disulfide, ester, ether, thioester, carbamate, carbonate, hydrazine, or amide linkages. In preferred embodiments, the attachment occurs via an appropriate spacer that provides an ester bond or an amide bond between the agent and the dendrimer depending on the desired release kinetics of the agent.

Linking moieties generally include one or more organic functional groups. Examples of suitable organic functional groups include secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)-G-), ureas (—NHCONH—; —NRCONH—; —NHCONR—, —NRCONR—), carbinols (—CHOH—, —CROH—), disulfide groups, hydrazones, hydrazides, ethers (—O—), and esters (—COO—, —CH₂O₂C—, CHRO₂C—), wherein R is an alkyl group, an aryl group, or a heterocyclic group. In general, the identity of the one or more organic functional groups within the linking moiety is chosen in view of the desired release rate of the agents. In addition, the one or more organic functional groups can be selected to facilitate the covalent attachment of the agents to the dendrimers.

### 2. Spacers

In certain embodiments, the linking moiety includes one or more of the organic functional groups described above in combination with a spacer group. The term “spacers” includes compositions used for linking a therapeutic, prophylactic and/or diagnostic agent to the dendrimer. The spacer can be either a single chemical entity or two or more chemical entities linked together to bridge the polymer and the therapeutic agent or imaging agent. The spacers can include any small chemical entity, peptide or polymers having sulfhydryl, thiopyridine, succinimidyl, maleimide, vinylsulfone, and carbonate terminations.

In some embodiments, the spacer group is composed of an assembly of atoms, including oligomeric and polymeric chains; however, the total number of atoms in the spacer group is preferably between 3 and 200 atoms, more preferably between 3 and 150 atoms, more preferably between 3 and 100 atoms, most preferably between 3 and 50 atoms. Examples of suitable spacer groups include alkyl groups, heteroalkyl groups, alkylaryl groups, oligo- and polyethylene glycol chains, and oligo- and poly(amino acid) chains. Variation of the spacer group provides additional control over the release of the agents in vivo. In embodiments where the linking moiety includes a spacer group, one or more organic functional groups will generally be used to connect the spacer group to both the therapeutic, prophylactic or diagnostic agent and the dendrimers. In some embodiments, the spacer is chosen from among a class of compounds terminating in sulfhydryl, thiopyridine, succinimidyl, maleimide, vinylsulfone and carbonate group. In some embodiments, the spacer includes thiopyridine terminated compounds such as dithiodipyridine, N-Succinimidyl 3-(2-pyridyldithio)-propionate (SPDP), Succinimidyl 6-(3-[2-pyridyldithio]-propionamido)hexanoate LC-SPDP or Sulfo-LC-SPDP. In some embodiments, the spacer includes peptides wherein the peptides are linear or cyclic, having sulfhydryl groups such as glutathione, homocysteine, cysteine and its derivatives, arg-gly-asp-cys (RGDC), cyclo(Arg-Gly-Asp-d-Phe-Cys) (c(RGDfC)), cyclo(Arg-Gly-Asp-D-Tyr-Cys), cyclo(Arg-Ala-Asp-d-Tyr-Cys). In some embodiments, the spacer is a mercapto acid derivative such as 3 mercapto propionic acid, mercapto acetic acid, 4 mercapto butyric acid, thiolan-2-one, 6 mercaptohexanoic acid, 5 mercapto valeric acid orother mercapto derivatives such as 2 mercaptoethanol and 2 mercaptoethylamine. In some embodiments, the spacer is thiosalicylic acid or its derivatives, (4-succinimidyloxycarbonyl-methyl-alpha-2-pyridylthio)toluene, (3-[2-pyridithio]propionyl hydrazide. In other embodiments, the spacer has maleimide terminations wherein the spacer includes polymer or small chemical entity such as bis-maleimido diethylene glycol and bis-maleimido triethylene glycol, bis-maleimidoethane, bismaleimidohexane. In some embodiments, the spacer includes vinylsulfone such as 1,6-Hexane-bis-vinylsulfone. In some embodiments, the spacer is a thioglycoside such as thioglucose. In some embodiments, the spacer is a reduced protein such as bovine serum albumin and human serum albumin, any thiol terminated compound capable of forming disulfide bonds. In some embodiments, the spacer includes polyethylene glycol having maleimide, succinimidyl and thiol terminations.

### C. Therapeutic, Prophylactic and Diagnostic Agents

The glucose dendrimers are complexed to, covalently conjugated to, or having intra-molecularly dispersed or encapsulated therein one or more therapeutic, prophylactic and/or diagnostic agents.

A wide range of agents may be included in the particles to be delivered. The agents can be proteins or peptides, sugars or carbohydrate, nucleic acids or oligonucleotides, lipids, small molecules, or combinations thereof. The nucleic acid can be an oligonucleotide encoding a protein, for example, a DNA expression cassette or an mRNA. Representative oligonucleotides include siRNAs, microRNAs, DNA, RNA, and aptamers. In some embodiments, the therapeutic, prophylactic or diagnostic agent is a therapeutic antibody. One or more types of therapeutic, prophylactic or diagnostic agents can be encapsulated, complexed, or conjugated to the dendrimer. For example, the dendrimer is conjugated to one or more NAC molecules via disulfide bridge and one or more antibodies via amide linkages.

Exemplary therapeutic agents include anti-inflammatory drugs, anti-proliferatives, chemotherapeutics, vasodilators, neuroactive agents, and anti-infective agents. In some embodiments, the dendrimer is linked to the targeting moiety, imaging agents, and/or therapeutic agents.

### 1. Therapeutic Agents

One or more therapeutic agents can be complexed with, covalently attached to or intra-molecularly dispersed or encapsulated within the dendrimer. In some embodiments, two or more different therapeutic agents can be associated, via covalent and/or non-covalent interactions, with the dendrimer.

The dendrimer conjugates, when administered by intravenous injection, can preferentially cross the blood brain barrier (BBB). Preferably the agent(s) is attached or conjugated to the dendrimers, which are capable of preferentially releasing the drug at the target site i.e., site of disease, and/or injury. For example, some drugs can be released intracellularly under the reduced conditions found in vivo. The dendrimer conjugates linked to an agent can be used to perform several functions including targeting, localization at a diseased site, releasing the drug, and imaging purposes. The dendrimer complexes can be tagged with or without targeting moieties.

In some embodiments, one or more therapeutic agents targeting the underlying cause of the disease or condition, and one or more therapeutic agents relieving one or more symptoms of the disease or condition.

Preferred therapeutic or prophylactic agents include agents that reduce neuroinflammation (e.g., N-acetyl cysteine, Pioglitazone, Vitamin E) and RNA oligonucleotides that interfere with gene transcription or translation. In particularly preferred embodiments, the agent is N-acetylcysteine, 4-phenylbutyrate, bezafibrate, thyroid hormone (T3), sobetirome, pioglitazone, resveratrol, VBP15, Vitamin E, erucic acid, Coenzyme Q10, clemastine, galactosylceramidase (GALC), Aspartoacylase (ASPA), or Arylsulfatase A (ARSA). Other suitable agents, include anti-inflammatory, neuroactive and imaging agents. The dendrimer can be conjugated to more than one agent and more than one type of agent.

a. Anti-Inflammatory Agents

In some embodiments, the compositions include one or more anti-inflammatory agents. Anti-inflammatory agents reduce inflammation and include steroidal and non-steroidal drugs.

A preferred anti-inflammatory is an antioxidant drug including N-acetylcysteine. Preferred NSAIDS include mefenamic acid, aspirin, diflunisal, salsalate, ibuprofen, naproxen, fenoprofen, ketoprofen, deacketoprofen, flurbiprofen, oxaprozin, loxoprofen, indomethacin, sulindac, etodolac, ketorolac, diclofenac, nabumetone, piroxicam, meloxicam, tenoxicam, droxicam, lornoxicam, isoxicam, meclofenamic acid, flufenamic acid, tolfenamic acid, elecoxib, rofecoxib, valdecoxib, parecoxib, lumiracoxib, etoricoxib, firocoxib, sulphonanilides, nimesulide, niflumic acid, and licofelone.

Representative small molecules include steroids such as methyl prednisone, dexamethasone, non-steroidal anti-inflammatory agents including COX-2 inhibitors, corticosteroid anti-inflammatory agents, gold compound anti-inflammatory agents, immunosuppressive, anti-inflammatory and anti-angiogenic agents, anti-excitotoxic agents such as valproic acid, D-aminophosphonovalerate, D-aminophosphonoheptanoate, inhibitors of glutamate formation/release, such as baclofen, NMDA receptor antagonists, salicylate anti-inflammatory agents, ranibizumab, anti-VEGF agents, including aflibercept, and rapamycin. Other anti-inflammatory drugs include nonsteroidal drug such as indomethacin, aspirin, acetaminophen, diclofenac sodium and ibuprofen. The corticosteroids can be fluocinolone acetonide and methylprednisolone.

Exemplary immune-modulating drugs include cyclosporine, tacrolimus and rapamycin. In some embodiments, anti-inflammatory agents are biologic drugs that block the action of one or more immune cell types such as T cells, or block proteins in the immune system, such as tumor necrosis factor-alpha (TNF-alpha), interleukin 17-A, interleukins 12 and 23.

In some embodiments, the anti-inflammatory drug is a synthetic or natural anti-inflammatory protein. Antibodies specific to select immune components can be added to immunosuppressive therapy. In some embodiments, the anti-inflammatory drug is an anti-T cell antibody (e.g., anti-thymocyte globulin or Anti-lymphocyte globulin), anti-IL-2Rα receptor antibody (e.g., basiliximab or daclizumab), or anti-CD20 antibody (e.g., rituximab).

In preferred embodiments, the one or more anti-inflammatory drugs are released from the dendrimeric conjugates after administration to a mammalian subject in an amount effective to inhibit inflammation for at least 1 day, 2 days, 3 days, 4 days, 5 days, 6 days, preferably at least a week, 2 weeks, or 3 weeks, more preferably at least a month, two months, three months, four months, five months, six months.

b. Neuroactive Agents

A number of drugs have been developed to interrupt, influence, or temporarily halt the glutamate excitotoxic cascade toward neuronal injury. One strategy is the “upstream” decrease of glutamate release. This category of drugs includes riluzole, lamotrigine, and lifarizine, which are sodium channel blockers. The commonly used nimodipine is a voltage-dependent channel (L-type) blocker. Some agents affect the sites of the coupled glutamate receptor. Some of these drugs include felbamate, ifenprodil, magnesium, memantine, and nitroglycerin. These “downstream” drugs attempt to influence such intracellular events as free radical formation, nitric oxide formation, proteolysis, endonuclease activity, and ICE-like protease formation (an important component in the process leading to programmed cell death, or apoptosis).

Agents for the treatment of neurodegenerative diseases are well known in the art and can vary based on the symptoms and disease to be treated. For example, conventional treatment for Parkinson's disease can include levodopa (usually combined with a dopa decarboxylase inhibitor or COMT inhibitor), a dopamine agonist, or an MAO-B inhibitor.

Treatment for Huntington's disease can include a dopamine blocker to help reduce abnormal behaviors and movements, or a drug such as amantadine and tetrabenazine to control movement, etc. Other drugs that help to reduce chorea include neuroleptics and benzodiazepines. Compounds such as amantadine or remacemide have shown positive results. Hypokinesia and rigidity, especially in juvenile cases, can be treated with anti-Parkinsonian drugs, and myoclonic hyperkinesia can be treated with valproic acid. Psychiatric symptoms can be treated with medications similar to those used in the general population. Selective serotonin reuptake inhibitors and mirtazapine have been recommended for depression, while atypical antipsychotic drugs are recommended for psychosis and behavioral problems.

Riluzole (RILUTEK©) (2-amino-6-(trifluoromethoxy) benzothiazole), an anti-excitotoxin, has yielded improved survival time in subjects with ALS. Other medications and interventions can reduce symptoms due to ALS. Some treatments improve quality of life, and a few appear to extend life. Common ALS-related therapies are reviewed in Gordon, Aging and Disease, 4(5):295-310 (2013), see, e.g., Table 1 therein. A number of other agents have been tested in one or more clinical trials with efficacies ranging from non-efficacious to promising. Exemplary agents are reviewed in Carlesi, et al., Archives Italiennes de Biologie, 149:151-167 (2011). For example, therapies may include an agent that reduces excitotoxicity such as talampanel (8-methyl-7H-1,3-dioxolo(2,3)benzodiazepine), a cephalosporin such as ceftriaxone, or memantine; an agent that reduces oxidative stress such as coenzyme Q10, manganoporphyrins, KNS-760704 [(6R)-4,5,6,7-tetrahydro-N6-propyl-2,6-benzothiazole-diamine dihydrochloride, RPPX], or edaravone (3-methyl-1-phenyl-2-pyrazolin-5-one, MCI-186); an agent that reduces apoptosis such as histone deacetylase (HDAC) inhibitors including valproic acid, TCH346 (Dibenzo(b,f)oxepin-10-ylmethyl-methylprop-2-ynylamine), minocycline, or tauroursodeoxycholic Acid (TUDCA); an agent that reduces neuroinflammation such as thalidomide and celastol; a neurotropic agent such as insulin-like growth factor 1 (IGF-1) or vascular endothelial growth factor (VEGF); a heat shock protein inducer such as arimoclomol; or an autophagy inducer such as rapamycin or lithium.

Treatment for Alzheimer's Disease can include, for example, an acetylcholinesterase inhibitor such as tacrine, rivastigmine, galantamine or donepezil; an NMDA receptor antagonist such as memantine; or an antipsychotic drug.

Treatment for Dementia with Lewy Bodies can include, for example, acetylcholinesterase inhibitors such as tacrine, rivastigmine, galantamine or donepezil; the N-methyl d-aspartate receptor antagonist memantine; dopaminergic therapy, for example, levodopa or selegiline; antipsychotics such as olanzapine or clozapine; REM disorder therapies such as clonazepam, melatonin, or quetiapine; anti-depression and antianxiety therapies such as selective serotonin reuptake inhibitors (citalopram, escitalopram, sertraline, paroxetine, etc.) or serotonin and noradrenaline reuptake inhibitors (venlafaxine, mirtazapine, and bupropion) (see, e.g., Macijauskiene, et al., Medicina (Kaunas), 48(1):1-8 (2012)).

Exemplary neuroprotective agents include, for example, glutamate antagonists, antioxidants, and NMDA receptor stimulants. Other neuroprotective agents and treatments include caspase inhibitors, trophic factors, anti-protein aggregation agents, therapeutic hypothermia, and erythropoietin.

Other common therapeutic, prophylactic or diagnostic agents for treating neurological dysfunction include amantadine and anticholinergics for treating motor symptoms, clozapine for treating psychosis, cholinesterase inhibitors for treating dementia, and modafinil for treating daytime sleepiness.

c. Anti-Infective Agents

Antibiotics include beta-lactams such as penicillin and ampicillin, cephalosporins such as cefuroxime, cefaclor, cephalexin, cephydroxil, cepfodoxime and proxetil, tetracycline antibiotics such as doxycycline and minocycline, macrolide antibiotics such as azithromycin, erythromycin, rapamycin and clarithromycin, fluoroquinolones such as ciprofloxacin, enrofloxacin, ofloxacin, gatifloxacin, levofloxacin and norfloxacin, tobramycin, colistin, or aztreonam as well as antibiotics which are known to possess anti-inflammatory activity, such as erythromycin, azithromycin, or clarithromycin.

### 2. Diagnostic Agents

In some cases, the agents delivered to the target cells or tissues via glucose dendrimer are diagnostic agents. Examples of diagnostic agents that can be delivered to the brain by glucose dendrimer conjugates include paramagnetic molecules, fluorescent compounds, magnetic molecules, and radionuclides, x-ray imaging agents, and contrast media. Glucose dendrimer conjugates can include agents useful for determining the location of administered compositions. Agents useful for this purpose include fluorescent tags, radionuclides and contrast agents.

Exemplary diagnostic agents include dyes, fluorescent dyes, near infra-red dyes, SPECT imaging agents, PET imaging agents and radioisotopes. Representative dyes include carbocyanine, indocarbocyanine, oxacarbocyanine, thuicarbocyanine and merocyanine, polymethine, coumarine, rhodamine, xanthene, fluorescein, boron-dipyrromethane (BODIPY), Cy5, Cy5.5, Cy7, VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IRDye 800CW, IRDye 800RS, IRDye 700DX, ADS780WS, ADS830WS, and ADS832WS.

Exemplary SPECT or PET imaging agents include chelators such as di-ethylene tri-amine penta-acetic acid (DTPA), 1,4,7,10-tetra-azacyclododecane-1,4,7,10-tetraacetic acid (DOTA), di-amine dithiols, activated mercaptoacetyl-glycyl-glycyl-gylcine (MAG3), and hydrazidonicotinamide (HYNIC).

Exemplary isotopes include Tc-94m, Tc-99m, In-111, Ga-67, Ga-68, Gd3+, Y-86, Y-90, Lu-177, Re-186, Re-188, Cu-64, Cu-67, Co-55, Co-57, F-18, Sc-47, Ac-225, Bi-213, Bi-212, Pb-212, Sm-153, Ho-166, and Dy-166.

In preferred embodiments, the dendrimer compositions include one or more radioisotopes suitable for positron emission tomography (PET) imaging. Exemplary positron-emitting radioisotopes include carbon-11 (¹¹C), copper-64 (⁶⁴Cu), nitrogen-13 (¹³N), oxygen-15 (¹⁵O), gallium-68 (⁶⁸Ga), and fluorine-18 (¹⁸F), e.g., 2-deoxy-2-¹⁸F-fluoro-β-D-glucose (¹⁸F-FDG).

In further embodiments, a singular glucose dendrimer conjugate composition can simultaneously treat and/or diagnose a disease or a condition at one or more locations in the body.

### D. Dendrimer Conjugates

The surface groups allow for the attachment of small molecules, imaging agents, and small biological agents such as siRNA regardless of the payload's charge or aqueous solubility. Glucose dendrimers can include one or more therapeutic or prophylactic agents complexed, covalently conjugated, or intra-molecularly dispersed or encapsulated with the dendrimer.

In some embodiments, one or more agents are covalently attached to one or more terminal groups of the glucose dendrimer. In some embodiments, glucose dendrimer conjugates include one or more therapeutic, prophylactic or diagnostic agents conjugated or complexed with the glucose dendrimer via one or more linking moieties. In further embodiments, the linking moieties incorporate or are conjugated with one or more spacer moieties. The linking and/or spacer moieties can be cleavable, for example, by exposure to the intracellular compartments of target cells in vivo. The therapeutic, prophylactic or diagnostic agent and/or targeting moiety can be either covalently attached or intra-molecularly dispersed or encapsulated. The glucose dendrimer is preferably a generation 2, generation 3, generation 4, generation 5, generation 6, and up to generation 10, having hydroxyl surface groups on the terminal glucose monosaccharides. In preferred embodiments, the glucose dendrimer is linked to agents via a spacer ending in disulfide, ester, or amide bonds.

The optimal drug loading will necessarily depend on many factors, including the choice of drug, dendrimer structure and size, and tissues to be treated. In some embodiments, the one or more therapeutic, prophylactic, or diagnostic agents are encapsulated, associated, and/or conjugated to the dendrimer at a concentration between about 0.01% and about 45%, inclusive; preferably between about 0.1% and about 30%, inclusive; between about 0.1% and about 20%, inclusive; between about 0.1% and about 10%, inclusive; between about 1% and about 10%, inclusive; between about 1% and about 5%, inclusive; between about 3% and about 20% by weight, inclusive; and between about 3% and about 10% by weight, inclusive. However, optimal drug loading for any given drug, dendrimer, and site of target can be identified by routine methods, such as those described.

In some embodiments, the conjugation of agents/linkers occurs via about 1%, 2%, 3%, 4%, or 5% of the total available surface functional groups, preferably hydroxyl groups, of the dendrimers prior to the conjugation. In other embodiments, the conjugation of agents/linkers occurs on less than 5%, less than 10%, less than 15%, less than 20%, less than 25%, less than 30%, less than 35%, less than 40%, less than 45%, less than 50%, less than 55%, less than 60%, less than 65%, less than 70%, less than 75% total available surface functional groups of the dendrimers prior to the conjugation and/or the modification with therapeutic, prophylactic or diagnostic agents. In preferred embodiments, dendrimer complexes retain an effective amount of surface functional groups for targeting to target cells, whilst conjugated to an effective amount of agents for treat, prevent, and/or image the disease or disorder.

Typically, dendrimer conjugates have a hydrodynamic volume in the nanometer range. For example, in some embodiments, the glucose dendrimer complex including one or more therapeutic, prophylactic or diagnostic agents complexed with or conjugated to the dendrimer has a diameter of about 2 nm to about 100 nm, or more than 100 nm, up to 500 nm, depending upon the generation of dendrimer, the chemical composition and amount of therapeutic, prophylactic or diagnostic agent loaded. Preferably, a glucose dendrimer conjugate including one or more therapeutic, prophylactic or diagnostic agents complexed with or conjugated to the dendrimer has a diameter effective to penetrate brain tissue and to retain in target cells for a prolonged period of time.

The presence of therapeutic, prophylactic or diagnostic agents can affect the zeta-potential or the surface charge of the dendrimer conjugates. In one embodiment, the zeta potential of the dendrimer conjugated or complexed with therapeutic, prophylactic or diagnostic agent(s) is between −100 mV and 100 mV, between −50 mV and 50 mV, between −25 mV and 25 mV, between −20 mV and 20 mV, between −10 mV and 10 mV, between −10 mV and 5 mV, between −5 mV and 5 mV, or between −2 mV and 2 mV. The range above is inclusive of all values from −100 mV to 100 mV. In a preferred embodiment, the surface charge is neutral or near-neutral, i.e., from about −10 mV to about 10 mV, inclusive.

### III. Methods of Making Glucose Dendrimers

Glucose dendrimers can be prepared via a variety of chemical reaction steps. Dendrimers are usually synthesized according to methods allowing for the control of the dendrimer structure at every stage. The dendrimeric structures are primarily synthesized by one of two different approaches: divergent or convergent.

In some embodiments, dendrimers are prepared using divergent methods, in which the dendrimer is assembled from a multifunctional core, which is extended outward by a series of reactions. The strategy involves the coupling of monomeric molecules that possesses reactive and protective groups with the multifunctional core moiety, which leads to stepwise addition of generations around the core followed by removal of protecting groups. For example, dendrimers are first synthesized by coupling AB₄ peracetylated β-D glucose-PEG4-azide monomers to hexapropargylated core.

In some embodiments, the core of the dendrimer, one or more branching units, one or more linkers/spacers, and/or one or more surface groups are modified to allow conjugation to further functional groups (branching units, linkers/spacers, surface groups, etc.), monomers, and/or agents via click chemistry, employing one or more Copper-Assisted Azide-Alkyne Cycloaddition (CuAAC), Diels-Alder reaction, thiol-ene and thiol-yne reactions, and azide-alkyne reactions (Arseneault M et al., Molecules. 2015 May 20; 20(5):9263-94). ‘Click chemistry’ involves the coupling of two different moieties (e.g., a core group and a branching unit; or a branching unit and a surface group) via a 1,3-dipolar cycloaddition reaction between an alkyne moiety (or equivalent thereof) on the surface of the first moiety and an azide moiety (e.g., present on a triazine composition or equivalent thereof), or any active end group such as, for example, a primary amine end group, a hydroxyl end group, a carboxylic acid end group, a thiol end group, etc.) on the second moiety.

In some embodiments, dendrimer synthesis relies upon one or more reactions such as thiol-ene click reactions, thiol-yne click reactions, CuAAC, Diels-Alder click reactions, azide-alkyne click reactions, Michael Addition, epoxy opening, esterification, silane chemistry, and a combination thereof.

In some embodiments, methods involve one or more protection and deprotection steps of the function groups (e.g., hydroxyl groups) on the central core, branching units, and/or therapeutic, prophylactic or diagnostic agents to facilitate addition of branching units to generate desired dendrimer molecules, or addition of therapeutic, prophylactic or diagnostic agents to generate desired dendrimer conjugates. In the case of hydroxyl groups, they may be protected by formation of an ether, an ester, or an acetal. Other exemplary protection groups include Boc and Fmoc.

### 1. Synthesis of Hypercore

In preferred embodiments, the hypercore is prepared from dipentaerythritol, for example by performing propargylation of dipentaerythritol to achieve the hexa-propargylated core.

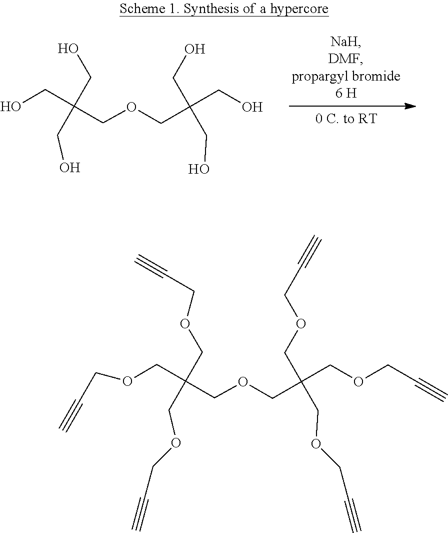 [SMILES: OCC(CO)(CO)COCC(CO)(CO)CO ; C#CCOCC(COCC#C)(COCC#C)COCC(COCC#C)(COCC#C)COCC#C]

### 2. Synthesis of Hypermonomer

In some embodiments, the branching units are hypermonomers i.e., ABₙ building blocks. Exemplary hypermonomers include AB₃, AB₄, AB₅, AB₆, AB₇, AB₅ building blocks. Hypermonomer strategy drastically increases the number of available end groups. An exemplary hypermonomer is AB₄ orthogonal hypermonomer including one azide functional group and four allyl groups prepared from dipentaerythritol with five allyl groups reacted with mono tosylated triethylene glycol azide (Scheme 2).

In some embodiments, the branching unit is polyethylene glycerol linear or branched as shown in Formula II. Other monomers include disaccharides and oligosaccharides, as well as sacchardides such as fructose, lactose, and sucrose.

a. Synthesis of AB₄ Building Block

Some exemplary synthesis methods of hypermonomer AB₄ are described below. In some embodiments, the hypermonomer AB₄ is based on glucose molecules. In preferred embodiments, the hypermonomer AB₄ is conjugated to a polyethylene glycerol, for example, tetraethylene glycol (PEG4). In one embodiment, the hypermonomer AB₄ is peracetylated β-D-Glucopyranoside tetraethylene glycol azide.

In some embodiments, the synthesis of glucose-OAc-TEG-OTs involves the following steps: a solution of peracetylated β-D-glucopyranoside (10 g, 25.6 mmol) was dissolved in 50 mL of anhydrous dichloromethane (DCM) followed by addition of 2-(2-(2-(2-hydroxyethoxy)ethoxy)ethoxy)ethyl 4-methylbenzenesulfonate (6.2 g, 17.9 mmol) and the reaction mixture was cooled to 0° C. Boron trifluoride diethyl etherate (2.5 eq.) was added and the reaction was allowed to come to room temperature. The reaction was monitored with the help of TLC and quenched after 5 hrs by the addition of saturated sodium bicarbonate solution at 0° C. After 10 minutes of stirring, DCM (300 mL) was added and the organic layer was washed with saturated sodium bicarbonate solution 3 times until the effervescence was quenched. The reaction mixture was dried over sodium sulfate, filtered, and evaporated under reduced pressure. The crude product was purified by combiflash chromatography using ethyl acetate/hexanes (70:30) mixture as eluents. The desired compound was achieved in 60% yield. Structure of glucose-OAc-TEG-OTs is shown below:

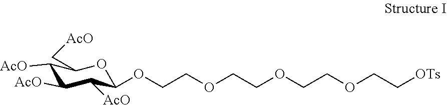

In some embodiments, the synthesis of glucose-OAc-TEG-N₃ involves the following steps: a solution of glucose-OAc-TEG-OTs (6 g, 8.8 mmoles) is dissolved in 40 mL of anhydrous DMF followed by the addition of sodium azide (2eq) and the reaction mixture is heated to 50° C. for overnight. Upon completion, the reaction mixture is filtered and DMF is evaporated. Once dried, the crude reaction mixture is passed through combiflash using ethyl acetate:hexane (70:30) as eluent. Structure of glucose-OAc-TEG-N3 is shown below:

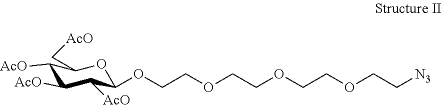

In some embodiments, the synthesis of glucose-OH-TEG-N₃ involves the following steps: the peracetylated β-D-Glucopyranoside tetraethylene glycol azide is dissolved in anhydrous methanol and sodium methoxide is added to adjust the pH around 8.5-9. The reaction is stirred overnight at room temperature, then diluted with methanol and pH is adjusted with Amberlist IR-120+ around 6-7. The reaction mixture is separated by filtration and the solvent removed by rotary evaporation. Structure of glucose-OH-TEG-N₃ is shown below.

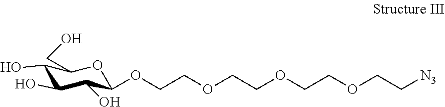

### 3. Synthesis of Glucose Dendrimers

In some embodiments, glucose dendrimers are synthesized by coupling AB₄ peracetylated j-D glucose-PEG4-azide monomers to hexapropargylated core as shown in FIG. 1A. In preferred embodiments, the hexapropargylated core (1) is linked to AB₄ β-D-glucose-PEG4-azide building block (2) via click reaction to obtain generation 1 dendrimer.

In some embodiments, generation one dendrimer D1-Glu6-OAc24 (compound 3a of FIG. 1A) is prepared according to the following: Hexapropargylated compound (0.5 g, 1 mmoles) and an azido derivative ((4.1 g, 7.4 mmoles) 1.2 eq. per acetylene) are suspended in a 1:1 mixture of DMF and water in a 20 mL microwave vial equipped with a magnetic stir bar. CuSO₄·5H₂O (5 mol %/acetylene, 75 mg) and sodium ascorbate (5 mol %/acetylene, 60 mg) dissolved in the minimum amount of water are added. The reaction is irradiated in a microwave at 50° C. for 6 h. The reaction mixture is dialyzed against DMF followed by water dialysis containing EDTA. The EDTA is further removed by extensive water dialysis. The product is lyophilized to obtain D1-Glu6-OAc24. Structure of D1-Glu6-OAc24 is shown below.

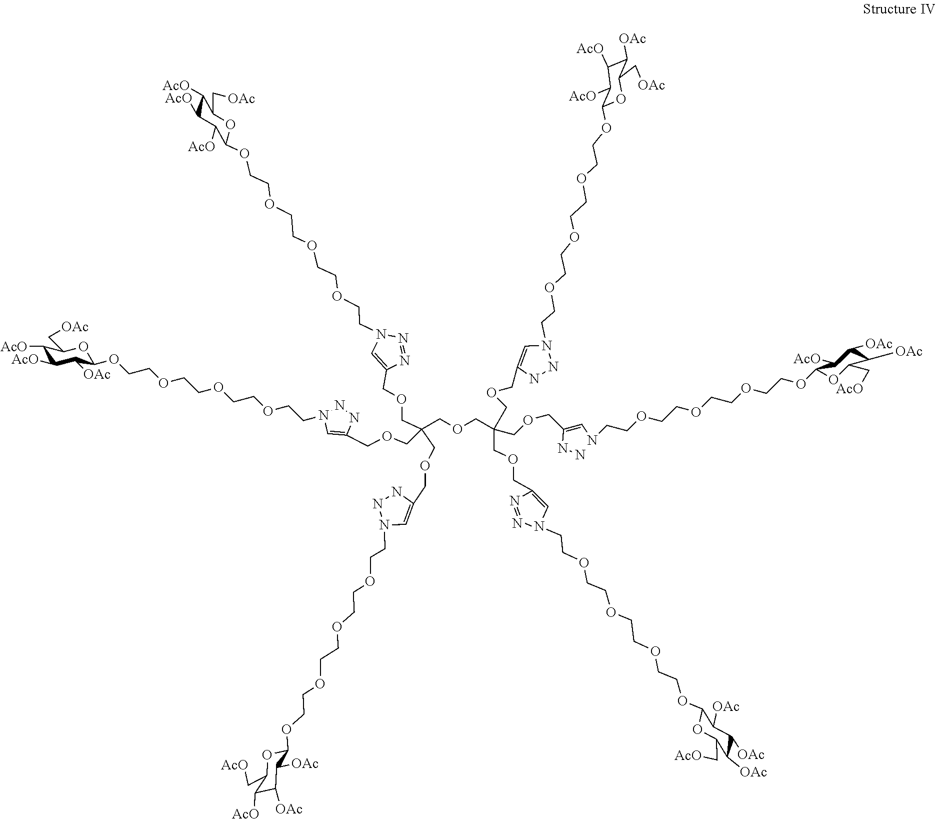

In some embodiments, generation one dendrimer D1-Glu₆-OH₂₄ (compound 3b of FIG. 1A) is prepared according to the following: the peracetylated generation 1 glucose dendrimer (1 g, 0.26 mmoles) is dissolved in anhydrous methanol and sodium methoxide is added to adjust the pH to around 8.5-9. The reaction is stirred overnight at room temperature, then diluted with methanol and pH is adjusted with AMBERLIST® IR-120+ around 6-7. The reaction mixture is separated by filtration and the solvent removed by rotary evaporation, followed by water dialysis. Structure of D1-Glu6-OH24 is shown below.

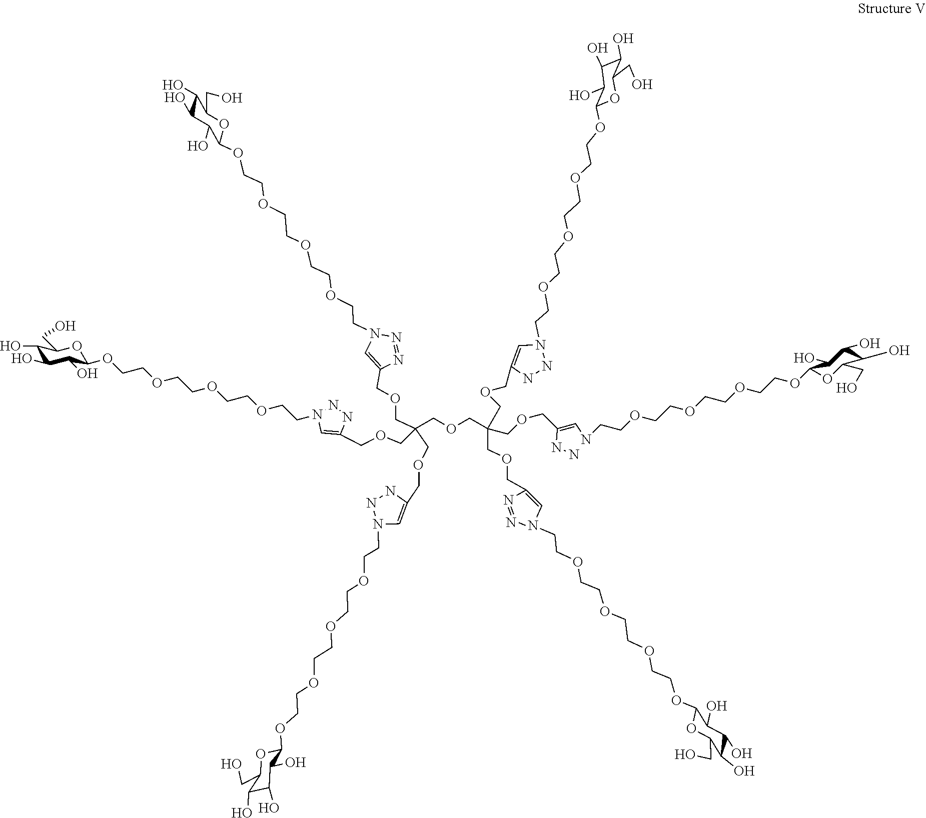

In some embodiments, generation one dendrimer D1-Glu6-OH24 is propargylated to provide D1-Acetylene24 (compound 4 of FIG. 1A) according to the following: D1-GLu6-OH24 (2 g, 0.721 mmol) was dissolved in anhydrous dimethylformamide (DMF, 50 mL) by sonication. Sodium hydride [160% dispersion in mineral oil](951 mg, 39.65 mmol) is slowly added in portions at 0° C. to the solution with stirring. The solution is stirred for an addition 15 minutes at 0° C. This is followed by the addition of propargyl bromide (3.85 mL, 34.608 mmol, 80% w/w solution in toluene) at 0° C. and the stirring is continued at room temperature for another 6 h. The reaction mixture is quenched with ice and water, filtered, and dialyzed against DMF, followed by the water dialysis to afford D1-acetylene24. Structure of D1-acetylene24 is shown below.

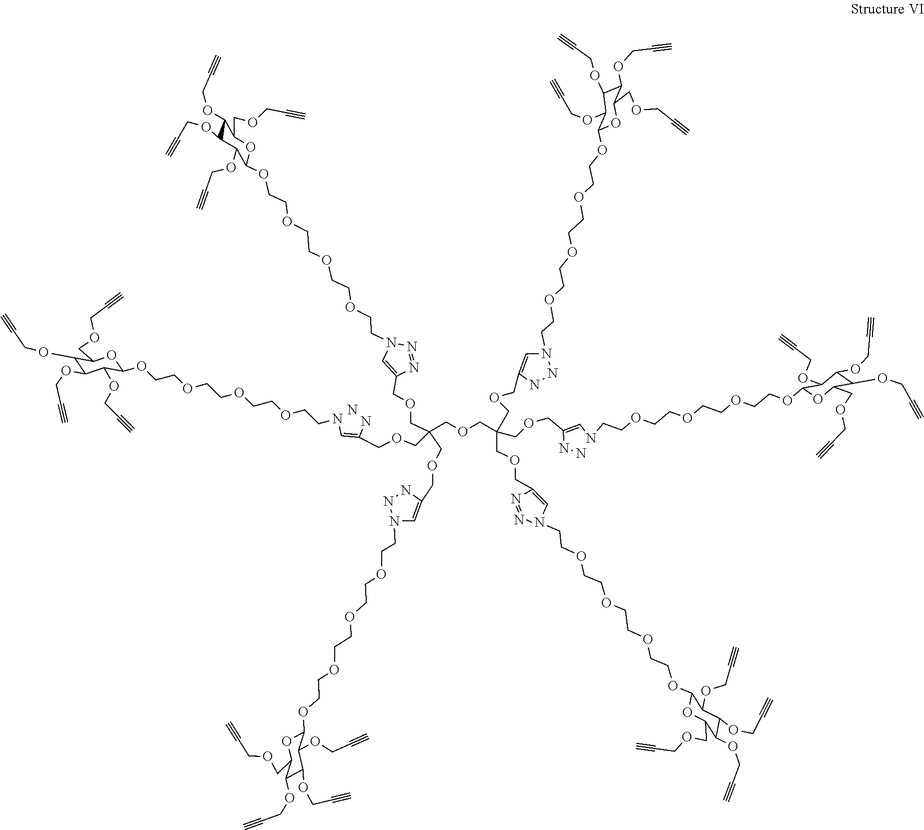

In some embodiments, generation one dendrimer D1-acetylene24 is further reacted with AB₄ β-D-glucose-PEG4-azide to provide generation 2 dendrimer with 24 glucose molecules containing 96 surface hydroxyl groups.

An exemplary generation two dendrimer D2-Glu24-OAc96 (compound 5a of FIG. 1A) is prepared according to the following: D1-acetylene dendrimer24 (0.5 g, 0.13 mmoles) and glucose-OAc-TEG-azide (2.2 g, 4 mmoles) are suspended in a 1:1 mixture of DMF and water in a 20 mL microwave vial equipped with a magnetic stir bar. To this CuSO₄·5H₂O (5 mol %/acetylene, 5 mg) and sodium ascorbate (5 mol %/acetylene, 10 mg) dissolved in the minimum amount of water are added. The reaction is irradiated in a microwave at 50° C. for 8 h. Upon completion, the reaction mixture is dialyzed against DMF followed by water dialysis containing EDTA. The EDTA is further removed by extensive water dialysis. The product is lyophilized to obtain D2-Glu24-OAc96.

In some embodiments, generation two dendrimer D2-Glu24-OH96 (compound 5b of FIG. 1A) is prepared according to the following: the peracetylated generation 2 glucose dendrimer D2-Glu24-OH96 is dissolved in anhydrous methanol and sodium methoxide is added to adjust the pH around 8.5-9.0. The reaction is stirred overnight at room temperature, then diluted with methanol and pH is adjusted with AMBERLIST® IR-120+ around 6-7. The reaction mixture is filtered to remove the resin and the filtrate is evaporated by rotary evaporation followed by water dialysis to obtain the product as off-white solid.

In some embodiments, generation two dendrimer D2-Glu24-OH96 is propargylated at one or more terminal hydroxyl groups suitable for further conjugation to one or more therapeutic, prophylactic or diagnostic agents. In some embodiments, one or more terminal hydroxyl groups of generation two dendrimer D2-Glu24-OH96 is propargylated according to the following: D2-Glu24-OH96 (5b) (200 mg, 0.016 mmol) is dissolved in anhydrous dimethylformamide (DMF, 10 mL) by sonication. To this stirring solution, sodium hydride [60% dispersion in mineral oil](22 mg, 0.934 mmol) is slowly added in portions at 0° C. The solution is additionally stirred for 15 minutes at 0° C. This is followed by the addition of propargyl bromide (18.0 μL, 80% w/w solution in toluene) at 0° C. and the stirring is continued at room temperature for another 6 h. The solvent is evaporated using V10 evaporator system and the crude product is purified by passing through PD10 SEPHADEX® G25 M column. The aqueous solution is lyophilized to afford the product as off-white solid.

In some embodiments, one or more fluorescent dyes such as infrared fluorescent Cy5 dyes are conjugated to generation two dendrimer D2-Glu24-OH96. In one embodiment, Cy5-D2-Glu24-OH96 (compound 7 of FIG. 1B) is prepared according to the following: Compound 6 (200 mg, 0.016 mmol) and Cy5 azide (20.7 mg, 0.02 mmol) are suspended in a 1:1 mixture of DMF and water in a 25 mL round bottom flask equipped with a magnetic stir bar. To this, CuSO₄·5H₂O (5 mol %/acetylene, 0.3 mg) and sodium ascorbate (10 mol %/acetylene, 0.5 mg) dissolved in the minimum amount of water are added. The reaction is stirred at room temperature for 24 h. Upon completion, the DMF is evaporated using V10 and the purification is performed using PD10 Sephadex G25 M column. The aqueous solution is lyophilized to afford the product as blue solid.

In some embodiments, the total hydroxyl groups for further conjugation to active agents including therapeutic and/or diagnostic agents are about 1-30, 2-20, or 5-10 out of total 96 available hydroxyl groups of the exemplary generation 2 dendrimer with 24 glucose molecules containing 96 surface hydroxyl groups.

### 4. Synthesis of Glucose Dendrimer Conjugates

Methods for conjugating agents with dendrimers are known in the art and for example, as described in U.S. Published Application Nos. US 2011/0034422, US 2012/0003155, and US 2013/0136697.

Reactions and strategies useful for the covalent attachment of agents to dendrimers are known in the art. See, for example, March, “Advanced Organic Chemistry,” 5th Edition, 2001, Wiley-Interscience Publication, New York) and Hermanson, “Bioconjugate Techniques,” 1996, Elsevier Academic Press, U.S.A. Appropriate methods for the covalent attachment of a given agent can be selected in view of the linking moiety desired, as well as the structure of the agents and dendrimers as it relates to compatibility of functional groups, protecting group strategies, and the presence of labile bonds.

In preferred embodiments, glucose dendrimer-drug conjugates are prepared using click chemistry. The click chemistry makes the synthesis facile and robust, thereby providing good control on synthesis and ligand loading (Sharma, R. et al., Journal of Controlled Release 2020; Sharma, R. et al., Bioconjugate Chemistry 2017, 28 (11), 2874-2886; Sharma, A. et al., Theranostics 2018, 8 (20), 5529). Techniques to obtain highly pure dendrimer drug conjugates using CuAAC chemistry have been published (Sharma, A. et al., Science Advances 2020, 6 (4), eaay8514; Sharma, R. et al., Biomacromolecules 2021, 22 (8), 3574-3589; Sharma, A. et al., Biomacromolecules 2020, 21 (12), 5148-5161; and Sharma, A. et al., Journal of Controlled Release 2018, 283, 175-189).

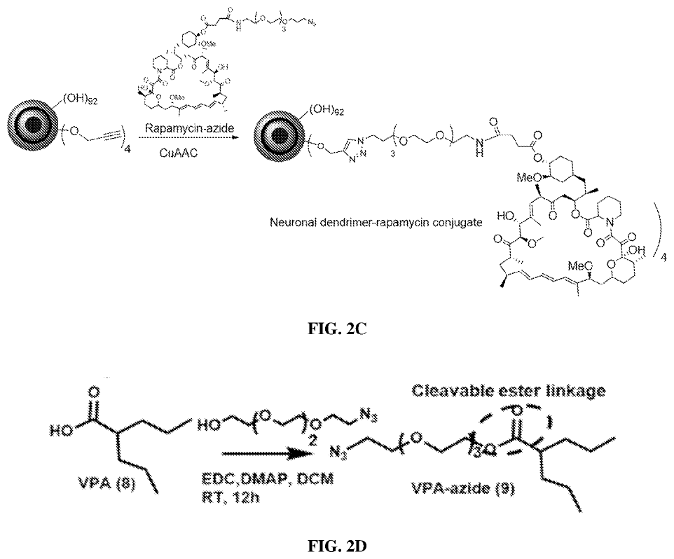

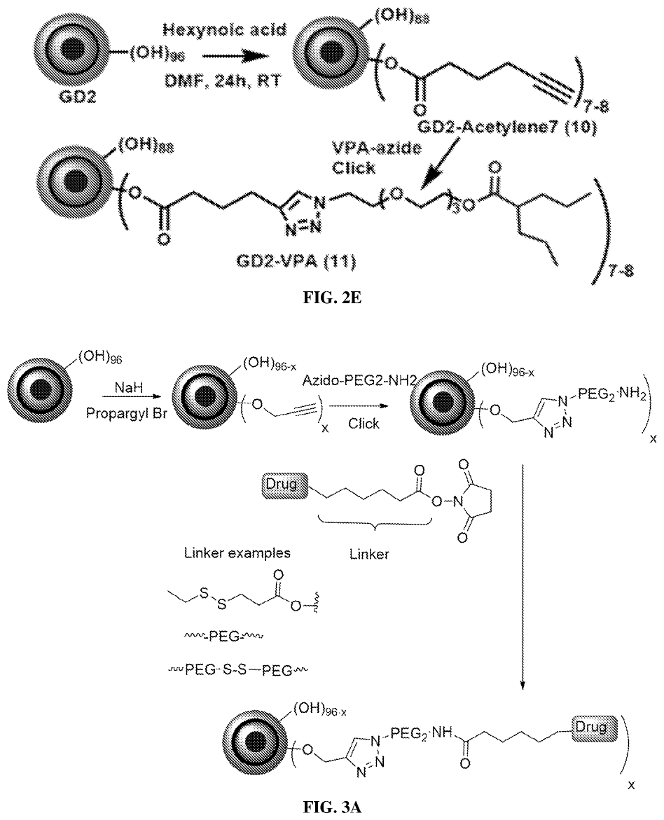

Exemplary glucose dendrimer conjugates are shown in FIGS. 2A-2C, 3A-3B, and 4. The structure confirmation and the purity of the conjugate can be determined by the ¹H NMR and HPLC respectively.

a. Synthesis Using Copper Catalyzed Alkyne-Azide Click (CuAAC)

In some embodiments, glucose dendrimer-drug conjugates are prepared using copper catalyzed alkyne-azide click (CuAAC). In some embodiments, the synthesis of glucose dendrimer drug conjugates begins with the partial modification of OH groups to bring propargyl groups (FIG. 2A). On the other hand, the drugs are modified using releasable or non-releasable chemical linkages to bring an azide terminal groups through polyethylene glycol linkers. An exemplary synthesis of glucose dendrimer-loperamide conjugate is shown in FIG. 2B: Loperamide hydrochloride is reacted with azido-PEG4-acid in the presence of DCC and DMAP in DCM at room temperature to afford loperamide-PEG-azide. On the other hand, partial modification of OH groups of the glucose dendrimer is carried out to bring approximately 10 propargyl groups, which are reacted with loperamide-PEG-azide to get glucose dendrimer-loperamide conjugate.

An exemplary synthesis of rapamycin-azide is achieved using the protocol of Sharma, A. et al., Biomacromolecules 2020, 21, (12), 5148-5161. On the other hand, partial modification of OH groups of glucose dendrimers is carried out to bring ˜4 propargyl groups, which are reacted with rapamycin-azide to get glucose dendrimer-r conjugate (FIG. 2C). The structure confirmation and the purity of the conjugate are achieved by the 1H NMR and HPLC, respectively.

b. Synthesis Using a Combination of Click and Esterification/Amidation Reactions

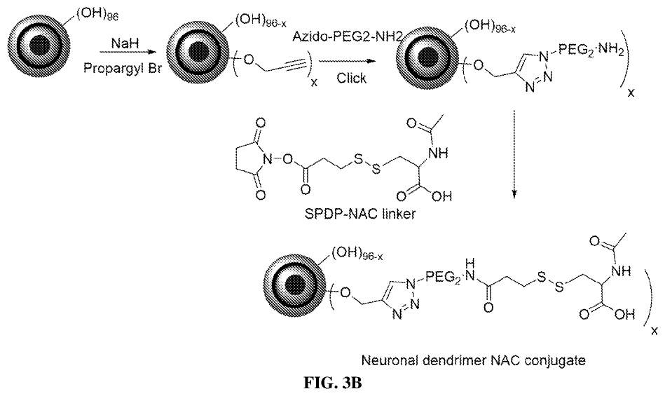

In some embodiments, glucose dendrimer-drug conjugates are prepared using a combination of click and esterification/amidation reactions. The synthesis is achieved by partial modification of hydroxyl (—OH) groups of glucose dendrimers is to bring propargyl groups, which are reacted with linker containing azide and amine termini to bring surface amine groups (FIG. 3A). The drug is modified using linker (hydrocarbon or PEG chains containing disulfide, ester, or amide linkages) with carboxylic acid, or —NHS ester terminal. The drug and dendrimer are then reacted using amidation reaction using coupling agents such as EDC and DMAP.

An exemplary synthesis of glucose dendrimer NAC conjugate is achieved by the partial modification of OH groups of glucose dendrimers to bring propargyl groups, which are reacted with linker containing azide and amine termini to bring surface amine groups (FIG. 3B). The SPDP-NAC is obtained by the previously published procedure. NAC-SPDP and dendrimer are the reacted at pH 7.4 to obtain glucose dendrimer NAC conjugate.

c. Synthesis Using Copper-Free Biorthogonal Click Chemistry

In some embodiments, glucose dendrimer-drug conjugates are prepared using copper-free biorthogonal click chemistry. In preferred embodiments, the copper-free click reactions such as TCO-triazine (FIG. 4), strain promoted azide-alkyne, Staudinger ligation, DBCO-azide click reactions are used to attach drugs, siRNA, peptides, mRNAs, oligonucleotides, antibodies, and other biologics, where presence of copper and reducing agent can reduce the effect of the therapeutic, prophylactic or diagnostic agent.

### IV. Pharmaceutical Formulations

Pharmaceutical compositions including glucose dendrimers and one or more therapeutic, prophylactic or diagnostic agents may be formulated in a conventional manner using one or more physiologically acceptable carriers, optionally including excipients and auxiliaries which facilitate processing of the active compounds into preparations which can be used pharmaceutically, for oral, intranasal, subcutaneous, intraperitoneal, or intramuscular administration.

Proper formulation is dependent upon the route of administration chosen. In preferred embodiments, the compositions are formulated for parenteral delivery. In some embodiments, the compositions are formulated for intravenous injection. Typically, the compositions will be formulated in sterile saline or buffered solution for injection into the tissues or cells to be treated. The compositions can be stored lyophilized in single use vials for rehydration immediately before use. Other means for rehydration and administration are known to those skilled in the art.

Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS) and may be administered to an individual without causing undesirable biological side effects or unwanted interactions.

Generally, pharmaceutically acceptable salts can be prepared by reaction of the free acid or base forms of an agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, MD, 2000, p. 704. Examples of ophthalmic drugs sometimes administered in the form of a pharmaceutically acceptable salt include timolol maleate, brimonidine tartrate, and sodium diclofenac.

The compositions of glucose dendrimer are preferably formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rabbits, dogs, or pigs. The animal model is also used to achieve a desirable concentration range and route of administration. Such information should then be useful to determine effective doses and routes for administration in humans. Therapeutic efficacy and toxicity of conjugates can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and is expressed as the ratio, LD₅₀/ED₅₀. Pharmaceutical compositions which exhibit large therapeutic indices are preferred. The data obtained from cell culture assays and animal studies can be used in formulating a range of dosages for human use.

In certain embodiments, the compositions of glucose dendrimer are administered locally, for example, by injection directly into a site to be treated. In some embodiments, the compositions are injected, topically applied, or otherwise administered directly into the vasculature onto vascular tissue at or adjacent to a site of injury, surgery, or implantation. For example, in embodiments, the compositions are topically applied to vascular tissue that is exposed, during a surgical procedure. Typically, local administration causes an increased localized concentration of the compositions, which is greater than that which can be achieved by systemic administration.

Pharmaceutical compositions of glucose dendrimer formulated for administration by parenteral (intramuscular, intraperitoneal, intravenous or subcutaneous injection) and enteral routes of administration are described.

### A. Parenteral Administration

The compositions of glucose dendrimer can be administered parenterally. The phrases “parenteral administration” and “administered parenterally” are art-recognized terms, and include modes of administration other than enteral and topical administration. The dendrimers can be administered orally, intranasally, subcutaneously, intraperitoneally, or intramuscularly. For liquid formulations, pharmaceutically acceptable carriers may be, for example, aqueous or non-aqueous solutions, suspensions, emulsions, or oils. Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media. The dendrimers can also be administered in an emulsion, for example, water in oil. Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Formulations suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, electrolyte replenishers such as those based on Ringer's dextrose. In general, water, saline, aqueous dextrose and related sugar solutions, and glycols such as propylene glycols or polyethylene glycol are preferred liquid carriers, particularly for injectable solutions.

Injectable pharmaceutical carriers for injectable compositions are well-known to those of ordinary skill in the art (see, e.g., Pharmaceutics and Pharmacy Practice, J. B. Lippincott Company, Philadelphia, PA, Banker and Chalmers, eds., pages 238-250 (1982), and ASHP Handbook on Injectable Drugs, Trissel, 15th ed., pages 622-630 (2009)).

### B. Enteral Administration

The compositions of glucose dendrimer can be administered enterally. The carriers or diluents may be solid carriers such as capsule or tablets or diluents for solid formulations, liquid carriers or diluents for liquid formulations, or mixtures thereof.

For liquid formulations, pharmaceutically acceptable carriers may be, for example, aqueous or non-aqueous solutions, suspensions, emulsions, or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Vehicles include, for example, sodium chloride solution, Ringer's dextrose, dextrose and sodium chloride, lactated Ringer's and fixed oils. Formulations include, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Vehicles can include, for example, fluid and nutrient replenishers, electrolyte replenishers such as those based on Ringer's dextrose. In general, water, saline, aqueous dextrose and related sugar solutions are preferred liquid carriers. These can also be formulated with proteins, fats, saccharides and other components of infant formulas.

In preferred embodiments, the compositions are formulated for oral administration. Oral formulations may be in the form of chewing gum, gel strips, tablets, capsules or lozenges. Encapsulating substances for the preparation of enteric-coated oral formulations include cellulose acetate phthalate, polyvinyl acetate phthalate, hydroxypropyl methylcellulose phthalate and methacrylic acid ester copolymers. Solid oral formulations such as capsules or tablets are preferred. Elixirs and syrups also are well known oral formulations.

### V. Methods of Use

Methods of using glucose dendrimer compositions are described. In preferred embodiments, the glucose dendrimer conjugates cross the blood brain barrier (BBB) and selectively target or enriched within neurons, preferably within the nucleus of neurons of injured/hyperactive neurons. In further embodiments, the glucose dendrimer conjugates also accumulate in activated microglia and astrocytes.

### A. Methods of Treatment

The formulations can be administered to treat disorders associated with infection, inflammation, or cancer, particular those having systemic inflammation that extends to the nervous system, especially the CNS and in the eye. More specifically, the formulations envisaged may be especially effective during early stages of brain injuries and ischemic injuries, where prevention of early neuronal death is critical.

Typically, an effective amount of dendrimer complexes including a combination of a dendrimer with one or more therapeutic, prophylactic, and/or diagnostic active agents are administered to an individual in need thereof. The dendrimers may also include a targeting agent, but as demonstrated by the examples, these are not required for delivery to neurons in the spinal cord and the brain and/or to retinal ganglion cells (RGCs) of the eye.

In some embodiments, the dendrimer conjugates are capable of releasing the therapeutic, prophylactic or diagnostic agents intracellularly under the conditions found in vivo. The amount of dendrimer conjugates administered to the subject is selected to deliver an effective amount to reduce, prevent, or otherwise alleviate one or more clinical or molecular symptoms of the disease or disorder to be treated compared to a control, for example, a subject treated with the therapeutic, prophylactic or diagnostic agent without dendrimer. In some embodiments, the methods including a step of selecting a subject who is likely to benefit from treatment with the glucose dendrimer compositions.

### B. Conditions to be Treated

The compositions are suitable for treating one or more diseases, conditions, and injuries in the eye, the brain, and the nervous system, particularly those associated with pathological activation of neurons, microglia and/or astrocytes. The compositions can also be used for treatment of ocular diseases and treatment of other tissues where the nerves play a role in the disease or disorder. The compositions and methods are also suitable for prophylactic use.

The dendrimer complex composition selectively targets neurons, which play a key role in the pathogenesis of many disorders and conditions including neurodevelopmental, neurodegenerative diseases, and brain cancer. Thus, the dendrimer complexes are administered in a dosage unit amount effective to treat or alleviate conditions associated with the pathological conditions of neurons. Generally, by targeting these cells, the dendrimers deliver agent specifically to treat diseased neurons.

In preferred embodiments, the dendrimers are administered in an amount effective to treat diseased neuron-mediated pathology in the subject in need thereof without any associated toxicity.

In some embodiments, the subject to be treated is a human. In some embodiments, the subject to be treated is a child, or an infant. All the methods can include the step of identifying and selecting a subject in need of treatment, or a subject who would benefit from administration with the described compositions.

### 1. Ocular Diseases and Injuries

The compositions and methods are suitable for treatment of discomfort, pain, dryness, excessive tearing, injuries, infections, burns associated with the eye.

Examples of eye disorders that may be treated include amoebic keratitis, fungal keratitis, bacterial keratitis, viral keratitis, onchorcercal keratitis, bacterial keratoconjunctivitis, viral keratoconjunctivitis, corneal dystrophic diseases, Fuchs' endothelial dystrophy, meibomian gland dysfunction, anterior and posterior blepharitis, conjunctival hyperemia, conjunctival necrosis, cicatrical scaring and fibrosis, punctate epithelial keratopathy, filamentary keratitis, corneal erosions, thinning, ulcerations and perforations, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, anterior uveitis, posterior uveitis (including toxoplasmosis), pan-uveitis, inflammatory disease of the vitreous or retina, endophthalmitis prophylaxis and treatment, macular edema, macular degeneration, age-related macular degeneration, proliferative and non-proliferative diabetic retinopathy, hypertensive retinopathy, an autoimmune disease of the retina, primary and metastatic intraocular melanoma, other intraocular metastatic tumors, glaucoma, open angle glaucoma, closed angle glaucoma, pigmentary glaucoma and combinations thereof. Other disorders include injury, burn, or abrasion of the cornea, cataracts and age-related degeneration of the eye or vision associated therewith.

In preferred embodiments, the eye disorder to be treated is age-related macular degeneration (AMD). Age-related macular degeneration (AMD) is a neurodegenerative, neuroinflammatory disease of the macula, which is responsible for central vision loss. The pathogenesis of age-related macular degeneration involves chronic neuroinflammation in the choroid (a blood vessel layer under the retina), the retinal pigment epithelium (RPE), a cell layer under the neurosensory retina, Bruch's membrane, and the neurosensory retina, itself.

### 2. Neurological and Neurodegenerative Diseases

The glucose dendrimer compositions and formulations thereof can be used to diagnose and/or to treat one or more neurological and neurodegenerative diseases. The compositions and methods are particularly suited for treating one or more neurological, or neurodegenerative diseases associated with defective or diseased neurons. In some embodiments, the disease or disorder is selected from, but not limited to, some psychiatric (e.g., depression, schizophrenia (SZ), alcohol use disorder, and morphine antinociceptive tolerance), neurological and neurodegenerative (e.g., Alzheimer's disease (AD), Parkinson disease (PD), Amyotrophic Lateral Sclerosis (ALS)) disorders. In one embodiment, the dendrimer complexes are used to treat Alzheimer's Disease (AD) or dementia.

Neurodegenerative diseases are chronic progressive disorders of the nervous system that affect neurological and behavioral function and involve biochemical changes leading to distinct histopathologic and clinical syndromes (Hardy H, et al., Science. 1998; 282:1075-9). Abnormal proteins resistant to cellular degradation mechanisms accumulate within the cells. The pattern of neuronal loss is selective in the sense that one group gets affected, whereas others remain intact. Often, there is no clear inciting event for the disease. The diseases classically described as neurodegenerative are Alzheimer's disease, Huntington's disease, and Parkinson's disease.

The compositions and methods can also be used to deliver agents for the treatment of a neurological or neurodegenerative disease or disorder or central nervous system disorder. In preferred embodiments, the compositions and methods are effective in treating, and/or alleviating neuroinflammation associated with a neurological or neurodegenerative disease or disorder or central nervous system disorder. The methods typically include administering to the subject an effective amount of the composition to increase cognition or reduce a decline in cognition, increase a cognitive function or reduce a decline in a cognitive function, increase memory or reduce a decline in memory, increase the ability or capacity to learn or reduce a decline in the ability or capacity to learn, or a combination thereof.

Neurodegeneration refers to the progressive loss of structure or function of neurons, including death of neurons. For example, the compositions and methods can be used to treat subjects with a disease or disorder, such as Parkinson's Disease (PD) and PD-related disorders, Huntington's Disease (HD), Amyotrophic Lateral Sclerosis (ALS), Alzheimer's Disease (AD) and other dementias, Prion Diseases such as Creutzfeldt-Jakob Disease, Corticobasal Degeneration, Frontotemporal Dementia, HIV-Related Cognitive Impairment, Mild Cognitive Impairment, Motor Neuron Diseases (MND), Spinocerebellar Ataxia (SCA), Spinal Muscular Atrophy (SMA), Friedreich's Ataxia, Lewy Body Disease, Alpers' Disease, neuronal ceroid lipofuscinoses, Batten Disease, Cerebro-Oculo-Facio-Skeletal Syndrome, Corticobasal Degeneration, Gerstmann-Straussler-Scheinker Disease, Kuru, Leigh's Disease, Monomelic Amyotrophy, Multiple System Atrophy, Multiple System Atrophy With Orthostatic Hypotension (Shy-Drager Syndrome), Multiple Sclerosis (MS), Duchenne muscular dystrophy (DMD), Neurodegeneration with Brain Iron Accumulation, Opsoclonus Myoclonus, Posterior Cortical Atrophy, Primary Progressive Aphasia, Progressive Supranuclear Palsy, Vascular Dementia, Progressive Multifocal Leukoencephalopathy, Dementia with Lewy Bodies (DLB), Lacunar syndromes, Hydrocephalus, Wernicke-Korsakoff's syndrome, post-encephalitic dementia, cancer and chemotherapy-associated cognitive impairment and dementia, and depression-induced dementia and pseudodementia.

Ina preferred embodiment, the disease or disorder is spinal muscular atrophy. In such cases, HDAC inhibitors, antisense oligonucleotide (ASO) drug nusinersen, or gene therapy drug ZOLGENSMA® can be conjugated to glucose dendrimers for delivery to neurons or nucleus of neurons for the treatment of spinal muscular atrophy.

In other embodiments, the disease or disorder is injection-localized amyloidosis, cerebral amyloid angiopathy, myopathy, neuropathy, brain trauma, frontotemporal dementia, Pick's disease, multiple sclerosis, prion disorders, diabetes mellitus type 2, fatal familial insomnia, cardiac arrhythmias, isolated atrial amyloidosis, atherosclerosis, rheumatoid arthritis, familial amyloid polyneuropathy, hereditary non-neuropathic systemic amyloidosis, Finnish amyloidosis, lattice corneal dystrophy, systemic AL amyloidosis, neuronopathic Gaucher disease, or Down syndrome. In preferred embodiments, the disease or disorder is Alzheimer's disease or dementia.

Criteria for assessing improvement in a particular neurological factor include methods of evaluating cognitive skills, motor skills, memory capacity or the like, as well as methods for assessing physical changes in selected areas of the central nervous system, such as magnetic resonance imaging (MRI) and computed tomography scans (CT) or other imaging methods. Such methods of evaluation are well known in the fields of medicine, neurology, psychology and the like, and can be appropriately selected to diagnosis the status of a particular neurological impairment. To assess a change in Alzheimer's disease, or related neurological changes, the selected assessment or evaluation test, or tests, are given prior to the start of administration of the dendrimer compositions. Following this initial assessment, treatment methods for the administration of the dendrimer compositions are initiated and continued for various time intervals. At a selected time interval subsequent to the initial assessment of the neurological defect impairment, the same assessment or evaluation test (s) is again used to reassess changes or improvements in selected neurological criteria.

a. Alzheimer's Disease and Dementia

The dendrimer compositions are suitable for reducing or preventing one or more pathological processes associated with the development and progression of neurological diseases such as Alzheimer's disease and dementia. Thus, methods for treatment, reduction, and prevention of the pathological processes associated with Alzheimer's disease include administering the dendrimer compositions in an amount and dosing regimen effective to reduce brain and/or serum exosomes, brain and/or serum ceramide levels, serum anti-ceramide IgG, glial activation, total A042 and plaque burden, tau phosphorylation/propagation, and improved cognition in a learning task, such as a fear-conditioned learning task, in an individual suffering from Alzheimer's disease or dementia are provided. Methods for reducing, preventing, or reversing the learning and/or memory deficits in an individual suffering from Alzheimer's disease or dementia are provided.

In some embodiments, the dendrimer compositions are administered in an amount and dosing regimen effective to induce neuro-enhancement in a subject in need thereof. Neuro-enhancement resulting from the administration of the dendrimer compositions includes the stimulation or induction of neural mitosis leading to the generation of new neurons, i.e., exhibiting a neurogenic effect, prevention or retardation of neural loss, including a decrease in the rate of neural loss, i.e., exhibiting a neuroprotective effect, or one or more of these modes of action. The term “neuroprotective effect” includes prevention, retardation, and/or termination of deterioration, impairment, or death of an individual's neurons, neurites, and neural networks. Administration of the compositions leads to an improvement, or enhancement, of neurological function in an individual with a neurological disease, neurological injury, or age-related neuronal decline or impairment.

Neural deterioration can be the result of any condition which compromises neural function which is likely to lead to neural loss. Neural function can be compromised by, for example, altered biochemistry, physiology, or anatomy of a neuron, including its neurite. Deterioration of a neuron may include membrane, dendritic, or synaptic changes, which are detrimental to normal neuronal functioning. The cause of the neuron deterioration, impairment, and/or death may be unknown. Alternatively, it may be the result of age-, injury- and/or disease-related neurological changes that occur in the nervous system of an individual.

In Alzheimer's patients, neural loss is most notable in the hippocampus, frontal, parietal, and anterior temporal cortices, amygdala, and the olfactory system. The most prominently affected zones of the hippocampus include the CA1 region, the subiculum, and the entorhinal cortex. Memory loss is considered the earliest and most representative cognitive change because the hippocampus is well known to play a crucial role in memory.

Neural loss through disease, age-related decline or physical insult leads to neurological disease and impairment. The compositions can counteract the deleterious effects of neural loss by promoting development of new neurons, new neurites and/or neural connections, resulting in the neuroprotection of existing neural cells, neurites and/or neural connections, or one or more these processes. Thus, the neuro-enhancing properties of the compositions provide an effective strategy to generally reverse the neural loss associated with degenerative diseases, aging and physical injury or trauma.

Administration of the glucose dendrimer compositions to an individual who is undergoing or has undergone neural loss, as a result of Alzheimer's disease reduces any one or more of the symptoms of Alzheimer's disease, or associated cognitive disorders, including dementia. Clinical symptoms of AD or dementia that can be treated, reduced or prevented include clinical symptoms of mild AD, moderate AD, and/or severe AD or dementia.

In mild Alzheimer's disease, a person may seem to be healthy but has more and more trouble making sense of the world around him or her. The realization that something is wrong often comes gradually to the person and their family. Exemplary symptoms of mild Alzheimer's disease/mild dementia include memory loss; poor judgment leading to bad decisions; loss of spontaneity and sense of initiative; taking longer to complete normal daily tasks; repeating questions; trouble handling money and paying bills; wandering and getting lost; losing things or misplacing them in odd places; mood and personality changes, and increased anxiety and/or aggression.

Symptoms of moderate Alzheimer's disease/moderate dementia include forgetfulness; increased memory loss and confusion; inability to learn new things; difficulty with language and problems with reading, writing, and working with numbers; difficulty organizing thoughts and thinking logically; shortened attention span; problems coping with new situations; difficulty carrying out multistep tasks, such as getting dressed; problems recognizing family and friends; hallucinations, delusions, and paranoia; impulsive behavior such as undressing at inappropriate times or places or using vulgar language; inappropriate outbursts of anger; restlessness, agitation, anxiety, tearfulness, wandering (especially in the late afternoon or evening); repetitive statements or movement, occasional muscle twitches.

Symptoms of severe Alzheimer's disease/severe dementia include inability to communicate; weight loss; seizures; skin infections; difficulty swallowing; groaning, moaning, or grunting; increased sleeping; loss of bowel and bladder control.

Physiological symptoms of Alzheimer's disease/dementia include reduction in brain mass, for example, reduction in hippocampal volume. Therefore, in some embodiments, methods of administering the dendrimer compositions to increase the brain mass, and/or reduce or prevent the rate of decrease in brain mass of a subject; increase the hippocampal volume of the subject, reduce or prevent the rate of decrease of hippocampal volume, as compared to an untreated control subject.

The dendrimer compositions are administered to provide an effective amount of one or more therapeutic agents upon administration to an individual. As used in this context, an “effective amount” of one or more therapeutic agents is an amount that is effective to improve or ameliorate one or more symptoms associated with Alzheimer's disease or dementia, including neurological defects or cognitive decline or impairment. Such a therapeutic effect is generally observed within about 12 to about 24 weeks of initiating administration of a composition containing an effective amount of one or more neuro-enhancing agents, although the therapeutic effect may be observed in less than 12 weeks or greater than 24 weeks.

The individual is preferably an adult human, and more preferably, a human over the age of 30, who has lost some amount of neurological function as a result of Alzheimer's disease or dementia. Generally, neural loss implies any neural loss at the cellular level, including loss in neurites, neural organization or neural networks.

In other embodiments, the methods including selecting a subject who is likely to benefit from treatment with the dendrimer compositions. For example, ceramide levels in the CSF of a patient are first determined and compared to that of a healthy control. In some embodiments, the dendrimer compositions are administered to a patient having an elevated concentration of ceramide in the CSF or in the serum relative to that of a healthy control. In other embodiments, the dendrimer compositions are administered to a patient with increased quantity of brain and/or serum exosomes relative to that of a healthy control. In other embodiments, the dendrimer compositions are administered to a patient with increased levels of serum anti-ceramide IgG relative to that of a healthy control.

In some embodiments, the subject has a nervous system disorder or is in need of neuroprotection. Exemplary conditions and/or subjects include, but are not limited to, subjects having had, subjects with, or subjects likely to develop or suffer from a stroke, a traumatic brain injury, a spinal cord injury, post-traumatic stress syndrome, or a combination thereof.

In some embodiments, the compositions and methods are administered to a subject in need thereof in an effective amount to reduce, or prevent one or more molecular or clinical symptoms of a neurodegenerative disease, or one or more mechanisms that cause neurodegeneration.

Agents for the treatment of neurodegenerative diseases are well known in the art and can vary based on the symptoms and disease to be treated. For example, conventional treatment for Parkinson's disease can include levodopa (usually combined with a dopa decarboxylase inhibitor or COMT inhibitor), a dopamine agonist, or an MAO-B inhibitor.

Treatment for Huntington's disease can include a dopamine blocker to help reduce abnormal behaviors and movements, or a drug such as amantadine and tetrabenazine to control movement, etc. Other drugs that help to reduce chorea include neuroleptics and benzodiazepines. Compounds such as amantadine or remacemide have shown preliminary positive results. Hypokinesia and rigidity, especially in juvenile cases, can be treated with antiparkinsonian drugs, and myoclonic hyperkinesia can be treated with valproic acid. Psychiatric symptoms can be treated with medications similar to those used in the general population. Selective serotonin reuptake inhibitors and mirtazapine have been recommended for depression, while atypical antipsychotic drugs are recommended for psychosis and behavioral problems.

Riluzole (RILUTEK®) (2-amino-6-(trifluoromethoxy) benzothiazole), an antiexcitotoxin, has yielded improved survival time in subjects with ALS. Other medications, most used off-label, and interventions can reduce symptoms due to ALS. Some treatments improve quality of life and a few appear to extend life. Common ALS-related therapies are reviewed in Gordon, Aging and Disease, 4(5):295-310 (2013), see, e.g., Table 1 therein. A number of other agents have been tested in one or more clinical trials with efficacies ranging from non-efficacious to promising. Exemplary agents are reviewed in Carlesi, et al., Archives Italiennes de Biologie, 149:151-167 (2011). For example, therapies may include an agent that reduces excitotoxicity such as talampanel (8-methyl-7H-1,3-dioxolo(2,3)benzodiazepine), a cephalosporin such as ceftriaxone, or memantine; an agent that reduces oxidative stress such as coenzyme Q10, manganoporphyrins, KNS-760704 [(6R)-4,5,6,7-tetrahydro-N6-propyl-2,6-benzothiazole-diamine dihydrochloride, RPPX], or edaravone (3-methyl-1-phenyl-2-pyrazolin-5-one, MCI-186); an agent that reduces apoptosis such as histone deacetylase (HDAC) inhibitors including valproic acid, TCH346 (Dibenzo(b,f)oxepin-10-ylmethyl-methylprop-2-ynylamine), minocycline, or tauroursodeoxycholic Acid (TUDCA); an agent that reduces neuroinflammation such as thalidomide and celastol; a neurotropic agent such as insulin-like growth factor 1 (IGF-1) or vascular endothelial growth factor (VEGF); a heat shock protein inducer such as arimoclomol; or an autophagy inducer such as rapamycin or lithium.

Treatment for Dementia with Lewy Bodies can include, for example, acetylcholinesterase inhibitors such as tacrine, rivastigmine, galantamine or donepezil; the N-methyl d-aspartate receptor antagonist memantine; dopaminergic therapy, for example, levodopa or selegiline; antipsychotics such as olanzapine or clozapine; REM disorder therapies such as clonazepam, melatonin, or quetiapine; anti-depression and antianxiety therapies such as selective serotonin reuptake inhibitors (citalopram, escitalopram, sertraline, paroxetine, etc.) or serotonin and noradrenaline reuptake inhibitors (venlafaxine, mirtazapine, and bupropion) (see, e.g., Macijauskiene, et al., Medicina (Kaunas), 48(1):1-8 (2012)).

Other common therapeutic, prophylactic or diagnostic agents for treating neurological dysfunction include amantadine and anticholinergics for treating motor symptoms, clozapine for treating psychosis, cholinesterase inhibitors for treating dementia, and modafinil for treating daytime sleepiness.

### 3. Neurodevelopmental Disorder

Neurodevelopmental disorder generally implies that the brain is not formed normally from the beginning. Abnormal regulation of fundamental neurodevelopmental processes may occur, or there may be disruption by insult that may take various forms. Autism and attention deficit hyperactivity disorder have been classically described as neurodevelopmental disorders.

Cerebral palsy (CP) is one of the most common pediatric neurological/neurodevelopmental disorder, currently estimated to affect approximately 2 to 3 per thousand live births (Kirby, R S et al., Research in Developmental Disabilities, 32, 462 (2011)). CP is recognized in early childhood and the condition persists throughout the life. The most common causes of CP include prematurity, hypoxia-ischemia and placental insufficiency, birth asphyxia and maternal-fetal inflammation (Dammann, O. Acta Pcediatrica 2007, 96, 6; Yoon, B H et al., American Journal of Obstetrics and Gynecology 2000, 182, 675; and O'Shea, T M et al., Journal of child neurology 2012, 27, 22). Although CP is heterogeneous in etiology and mechanism of disease is very complex, however, neuroinflammation is a common pathophysiologic mechanism that is involved irrespective of the etiology. Targeting neuroinflammation and delivering drugs directly at the injured site can be beneficial.

The compositions and methods can also be used to deliver therapeutic, prophylactic or diagnostic agents for the treatment of a neurodevelopmental disorder, such as cerebral palsy. In preferred embodiments, the compositions and methods are effective in treating, and/or alleviating neuroinflammation associated with a neurodevelopmental disorder, such as cerebral palsy.

In some embodiments, the dendrimer complexes are effective to treat, image, and/or prevent inflammation of the brain in neurodevelopmental disorders, including, for example Rett syndrome. In a preferred embodiment, the dendrimer complex would be used to deliver an anti-inflammatory agent (D-NAC) and anti-excitotoxic and D-anti-glutamate agents. Preferred candidates are: MK801, Memantine, 1-MT.

In some embodiments, the dendrimer complexes are effective to treat, image, and/or prevent inflammation of the brain in autism spectrum disorders. The term “spectrum” refers to the wide range of symptoms, skills, and levels of impairment or disability that children with ASD can have. Some children are mildly impaired by their symptoms, while others are severely disabled. The latest edition of the Diagnostic and Statistical Manual of Mental Disorders (DSM-5) no longer includes Asperger's syndrome; although the characteristics of Asperger's syndrome are included within the broader category of ASD.

At this time, the only medications approved by the FDA to treat aspects of ASD are the antipsychotics risperidone (Risperdal) and aripripazole (Abilify). Some medications that may be prescribed off-label for children with ASD include the following:

Antipsychotic medications are more commonly used to treat serious mental illnesses such as schizophrenia. These medicines may help reduce aggression and other serious behavioral problems in children, including children with ASD. They may also help reduce repetitive behaviors, hyperactivity, and attention problems.

Antidepressant medications, such as fluoxetine or sertraline, are usually prescribed to treat depression and anxiety but are sometimes prescribed to reduce repetitive behaviors. Some antidepressants may also help control aggression and anxiety in children with ASD.

Stimulant medications, such as methylphenidate (RITALIN®), are safe and effective in treating people with attention deficit hyperactivity disorder (ADHD). Methylphenidate has been shown to effectively treat hyperactivity in children with ASD as well. But not as many children with ASD respond to treatment, and those who do have shown more side effects than children with ADHD and not ASD.

### 4. Brain Tumors

The compositions and methods should be useful for treating subjects having benign or malignant tumors by delaying or inhibiting the growth of a tumor in a subject, reducing the growth or size of the tumor, inhibiting, or reducing metastasis of the tumor, and/or inhibiting or reducing symptoms associated with tumor development or growth. In preferred embodiments, the tumors to be treated are neuronal and mixed neuronal-glial tumors. Neuronal and mixed neuronal-glial tumors are types of rare tumors that occur in the brain or spinal cord. In most cases the tumor is not cancerous (benign), but a tumor can press on nearby brain tissue and cause problems such as seizures.

Thus, glucose dendrimer conjugates can be administered in combination with one or more additional therapeutically active agents, which are known to be capable of treating brain tumors or one or more symptoms associated therewith.

For example, the dendrimers may be administered to the brain via intravenous administration or during surgery to remove all or a part of the tumor. The dendrimers may be used to deliver chemotherapeutic agents, agents to enhance adjunct therapy such as of a subject undergoing radiation therapy, wherein the hydroxyl-terminated dendrimers are covalently linked to at least one radiosensitizing agent, in an amount effective to suppress or inhibit the activity of DDX3 in the proliferative disease in the brain.

It will be understood by those of ordinary skill in the art, that in addition to chemotherapy, surgical intervention and radiation therapy are also used in treatment of cancers of the nervous system. Radiation therapy means administering ionizing radiation to the subject in proximity to the location of the cancer in the subject. In some embodiments, the radiosensitizing agent is administered in two or more doses and subsequently, ionizing radiation is administered to the subject in proximity to the location of the cancer in the subject. In further embodiments, the administration of the radiosensitizing agent followed by the ionizing radiation can be repeated for 2 or more cycles.

Typically, the dose of ionizing radiation varies with the size and location of the tumor, but is dose is in the range of 0.1 Gy to about 30 Gy, preferably in a range of 5 Gy to about 25 Gy.

In some embodiments, the ionizing radiation is in the form of sterotactic ablative radiotherapy (SABR) or sterotactic body radiation therapy (SBRT).

### C. Dosage and Effective Amounts

Dosage and dosing regimens are dependent on the severity and location of the disorder or injury and/or methods of administration, as well as the therapeutic or prophylactic agent being delivered. This can be determined by those skilled in the art. A therapeutically effective amount of the glucose dendrimer composition used in the treatment of a proliferative disease or disorder in the brain is typically sufficient to reduce or alleviate one or more symptoms of brain cancer and/or proliferative disorder in the brain. Typically, doses would be in the range from microgram/kg up to about 100 mg/kg of body weight.

Preferably, the therapeutic, prophylactic or diagnostic agents do not target or otherwise modulate the activity or quantity of healthy cells not within or associated with the diseased/damaged tissue, or do so at a reduced level compared to cells associated with a disease or disorder such as a cancer and/or proliferative disorder. In this way, by-products and other side effects associated with the compositions are reduced. Therefore, in preferred embodiments, glucose dendrimer compositions are administered in an amount that leads to an improvement, or enhancement, function in an individual with a disease or disorder, such as a cancer and/or proliferative disorder.

The actual effective amounts of glucose dendrimer composition can vary according to factors including the specific agent administered, the particular composition formulated, the mode of administration, and the age, weight, condition of the subject being treated, as well as the route of administration and the disease or disorder. Generally, for intravenous injection or infusion, the dosage will be lower than for oral administration.

Dosage can vary and can be administered in one or more dose administrations daily, for one or several days. Guidance can be found in the literature for appropriate dosages for given classes of pharmaceutical products. Optimal dosing schedules can be calculated from measurements of drug accumulation in the body of the subject or patient. Persons of ordinary skill in the art can easily determine optimum dosages, dosing methodologies and repetition rates. Optimum dosages can vary depending on the relative potency of individual pharmaceutical compositions, and can generally be estimated based on EC₅₀s found to be effective in in vitro and in vivo animal models.

Dosage forms of the pharmaceutical composition including the dendrimer compositions are also provided. “Dosage form” refers to the physical form of a dose of a therapeutic compound, such as a capsule or vial, intended to be administered to a patient. The term “dosage unit” refers to the amount of the therapeutic compounds to be administered to a patient in a single dose.

In general, the timing and frequency of administration will be adjusted to balance the efficacy of a given treatment or diagnostic schedule with the side effects of the given delivery system. Exemplary dosing frequencies include continuous infusion, single and multiple administrations such as hourly, daily, weekly, monthly, or yearly dosing.

In some embodiments, dosages are administered daily, biweekly, weekly, every two weeks or less frequently in an amount to provide a therapeutically effective increase in the blood level of the therapeutic agent. Where the administration is by other than an oral route, the compositions may be delivered over a period of more than one hour, e.g., 3-10 hours, to produce a therapeutically effective dose within a 24-hour period. Alternatively, the compositions can be formulated for controlled release, wherein the composition is administered as a single dose that is repeated on a regimen of once a week, or less frequently.

It will be understood by those of ordinary skill that a dosing regimen can be any length of time sufficient to treat the disorder in the subject. In some embodiments, the regimen includes one or more cycles of a round of therapy followed by a drug holiday (e.g., no drug). The drug holiday can be 1, 2, 3, 4, 5, 6, or 7 days; or 1, 2, 3, 4 weeks, or 1, 2, 3, 4, 5, or 6 months.

In preferred embodiments, methods for treating or preventing one or more symptoms of an injury, a disorder, or a disease in the brain/CNS of a subject in need thereof include administering to the subject a formulation including glucose dendrimers complexed to, covalently conjugated to, or having intra-molecularly dispersed or encapsulated therein one or more therapeutic or prophylactic agents in an amount effective to treat or prevent one or more symptoms of the injury, disorder, or disease in the brain/CNS of the subject. It will be understood by those of ordinary skill that a dosing regimen will be for an amount and for a length of time sufficient to treat an injury, a disorder, or a disease in the brain/CNS to alleviate one or more symptoms such as swelling, pain, or seizures. Physicians routinely determine the length and amounts of therapy to be administered. Typically, the glucose dendrimer conjugates including the one or more therapeutic, prophylactic or diagnostic agents are administered systemically, and are transported across the blood-brain-barrier (BBB) to enter the brain and are selectively taken up by injured and/or diseased neurons. Typically, the glucose dendrimer conjugates accumulate within nucleus of the neurons and deliver the therapeutic, prophylactic or diagnostic agents to these cells. The accumulation of glucose dendrimer conjugates in neurons is up to 100 times that of dendrimer conjugates without glucose-based monosaccharide branching units such as PAMAM. Therefore, in some embodiments, the effective amount of therapeutic, prophylactic or diagnostic agent required for treatment or prevention of an injury, a disorder, or a disease in the brain/CNS is up to one hundredth (100 times less) than the amount required when using PAMAM dendrimer conjugates, or the therapeutic, prophylactic or diagnostic agent alone, for example one quarter, one half, one fifth, one tenth, one twentieth, on thirtieth, one fortieth, one fiftieth, one sixtieth, one seventieth, one eightieth, one ninetieth, or one hundredth of the amount required when using PAMAM dendrimer conjugates, or the therapeutic, prophylactic or diagnostic agent alone.

### D. Combination Therapies and Procedures

The glucose dendrimer compositions can be administered alone or in combination with one or more conventional therapies. In some embodiments, the conventional therapy includes administration of one or more of the compositions in combination with one or more additional therapeutic, prophylactic or diagnostic agents. The combination therapies can include administration of the therapeutic, prophylactic or diagnostic agents together in the same admixture, or in separate admixtures. Therefore, in some embodiments, the pharmaceutical composition contains more than one therapeutic, prophylactic or diagnostic agent. Such formulations typically include an effective amount of an agent targeting the site of treatment. The additional therapeutic, prophylactic or diagnostic agent(s) can have the same or different mechanisms of action. In some embodiments, the combination results in an additive effect on the treatment of the disease or condition. In some embodiments, the combinations result in a more than additive effect on the treatment of the disease or disorder.

In some embodiments, the glucose dendrimer composition is administered prior to, in conjunction with, subsequent to, or in alternation with, treatment with one or more additional therapies or procedures. In some embodiments, the additional therapy is performed between drug cycles or during a drug holiday that is part of the composition dosage regime. For example, in some embodiments, the additional therapy or procedure is surgery, a radiation therapy, or chemotherapy. Examples of preferred additional therapeutic agents include other conventional therapies known in the art for treating the desired disease, disorder or condition.

In the context of Alzheimer's disease, the other therapeutic agents can include one or more of acetylcholinesterase inhibitors (such as tacrine, rivastigmine, galantamine or donepezil), beta-secretase inhibitors such as JNJ-54861911, antibodies such as aducanumab, agonists for the 5-HT2A receptor such as pimavanserin, sargramostim, AADvacl, CAD106, CNP520, gantenerumab, solanezumab, and memantine.

In the context of Dementia with Lewy Bodies, the other therapeutic agents can include one or more of acetylcholinesterase inhibitors such as tacrine, rivastigmine, galantamine or donepezil; the N-methyl d-aspartate receptor antagonist memantine; dopaminergic therapy, for example, levodopa or selegiline; antipsychotics such as olanzapine or clozapine; REM disorder therapies such as clonazepam, melatonin, or quetiapine; anti-depression and antianxiety therapies such as selective serotonin reuptake inhibitors (citalopram, escitalopram, sertraline, paroxetine, etc.) or serotonin and noradrenaline reuptake inhibitors (venlafaxine, mirtazapine, and bupropion) (see, e.g., Macijauskiene, et al., Medicina (Kaunas), 48(1):1-8 (2012)).

Exemplary neuroprotective agents are also known in the art in include, for example, glutamate antagonists, antioxidants, and NMDA receptor stimulants. Other neuroprotective agents and treatments include caspase inhibitors, trophic factors, anti-protein aggregation agents, therapeutic hypothermia, and erythropoietin.

Other common therapeutic, prophylactic or diagnostic agents for treating neurological dysfunction include amantadine and anticholinergics for treating motor symptoms, clozapine for treating psychosis, cholinesterase inhibitors for treating dementia, and modafinil for treating daytime sleepiness.

In the context of cancer treatment, the other therapies include one or more of conventional chemotherapy, inhibition of checkpoint proteins, adoptive T cell therapy, radiation therapy, and surgical removal of tumors.

### E. Controls

The therapeutic result of the glucose dendrimer compositions including one or more therapeutic, prophylactic or diagnostic agents can be compared to a control. Suitable controls are known in the art and include, for example, an untreated subject, or a placebo-treated subject. A typical control is a comparison of a condition or symptom of a subject prior to and after administration of glucose dendrimer compositions. The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known the art. In some embodiments, an untreated control subject suffers from the same disease or condition as the treated subject.

In some embodiments, a control includes an equivalent amount of therapeutic, prophylactic or diagnostic agent delivered alone, or bound to dendrimers without glucose-based branching units such as PAMAM dendrimers of a similar generation, molecular weight, and/or surface hydroxyl density.

### VI. Kits

The compositions can be packaged in kit. The kit can include a single dose or a plurality of doses of a composition including one or more therapeutic, prophylactic or diagnostic agents, encapsulated in, associated with, or conjugated to a dendrimer (e.g., one or more glucose dendrimers as described in the Examples), and instructions for administering the compositions. Specifically, the instructions direct that an effective amount of the dendrimer composition be administered to an individual with a particular disease/disorder as indicated. The composition can be formulated as described above with reference to a particular treatment method and can be packaged in any convenient manner.

The present invention will be further understood by reference to the following non-limiting examples.

### EXAMPLES

### Example 1: Synthesis of Glucose Dendrimer

### Materials and Methods

### Synthesis of AB₄ Building Block

Synthesis of glucose-OAc-TEG-OTs: A solution of peracetylated β-D-glucopyranoside (10 g, 25.6 mmol) was dissolved in 50 mL of anhydrous dichloromethane (DCM) followed by addition of 2-(2-(2-(2-hydroxyethoxy)ethoxy)ethoxy)ethyl 4-methylbenzenesulfonate (6.2 g, 17.9 mmol) and the reaction mixture was cooled to 0° C. Boron trifluoride diethyl etherate (2.5 eq.) was added and the reaction was allowed to come to room temperature. The reaction was monitored with the help of TLC and quenched after 5 hrs by the addition of saturated sodium bicarbonate solution at 0° C. After 10 minutes of stirring, DCM (300 mL) was added and the organic layer was washed with saturated sodium bicarbonate solution 3 times until the effervescence was quenched. The reaction mixture was dried over sodium sulfate, filtered, and evaporated under reduced pressure. The crude product was purified by combiflash chromatography using ethyl acetate/hexanes (70:30) mixture as eluents. The desired compound was achieved in 60% yield. Structure of glucose-OAc-TEG-OTs is shown below:

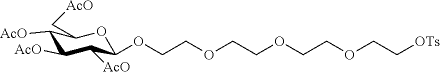

Synthesis of glucose-OAc-TEG-N3: A solution of glucose-OAc-TEG-OTs (6 g, 8.8 mmoles) was dissolved in 40 mL of anhydrous DMF followed by the addition of sodium azide (2eq) and the reaction mixture was heated to 50° C. for overnight. Upon completion, the reaction mixture is filtered and DMF was evaporated. Once dried, pass the crude reaction mixture through combiflash using ethyl acetate:hexane (70:30) as eluent. Structure of glucose-OAc-TEG-N₃ is shown below:

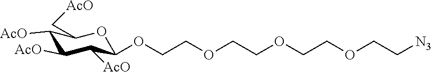

Synthesis of glucose-OH-TEG-N3: The peracetylated f-D-Glucopyranoside tetraethylene glycol azide was dissolved in anhydrous methanol and sodium methoxide was added to adjust the pH around 8.5-9. The reaction was stirred overnight at room temperature, then diluted with methanol and pH was adjusted with AMBERLIST® IR-120+ around 6-7. The reaction mixture was separated by filtration and the solvent removed by rotary evaporation. Structure of glucose-OH-TEG-N₃ is shown below.

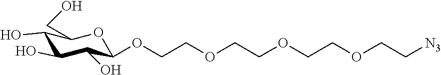

Synthesis of D1-Glu6-OAc24 (compound 3a): Hexapropargylated compound (0.5 g, 1 mmoles) and an azido derivative ((4.1 g, 7.4 mmoles) 1.2 eq. per acetylene) were suspended in a 1:1 mixture of DMF and water in a 20 mL microwave vial equipped with a magnetic stir bar. To this were added CuSO₄·5H₂O (5 mol %/acetylene, 75 mg) and sodium ascorbate (5 mol %/acetylene, 60 mg) dissolved in the minimum amount of water. The reaction was irradiated in a microwave at 50° C. for 6 h. The reaction mixture was dialyzed against DMF followed by water dialysis containing EDTA. The EDTA was further removed by extensive water dialysis. The product was lyophilized to obtain D1-Glu6-OAc24. Structure of D1-Glu6-OAc24 is shown below.

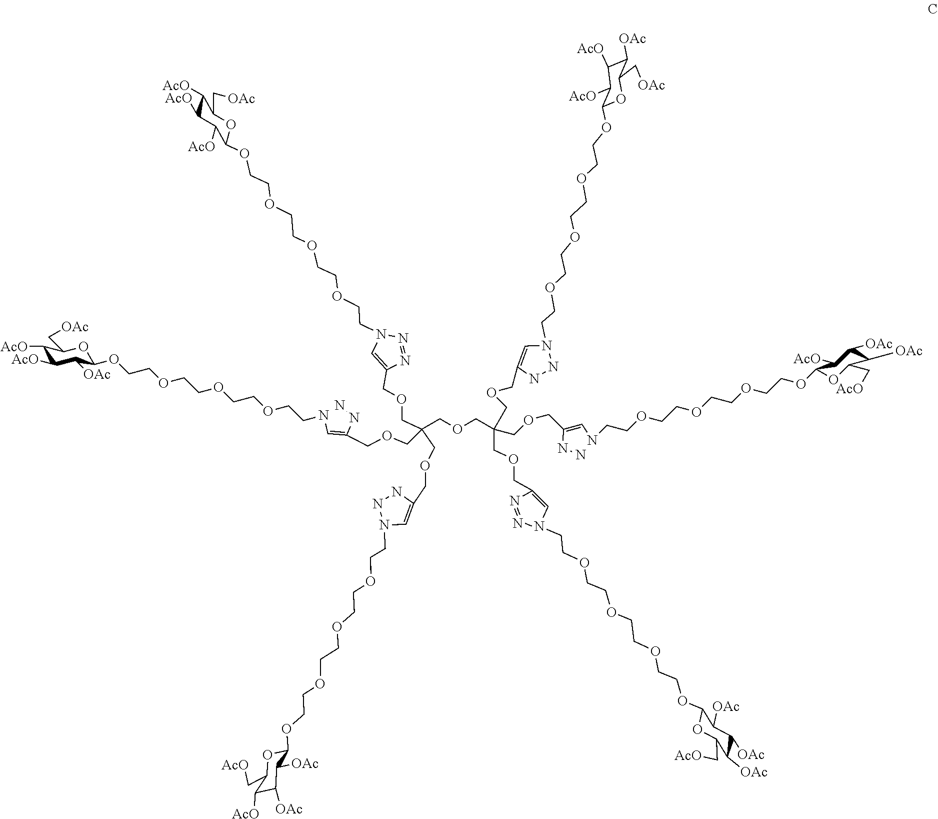

Synthesis of D1-Glu6-OH24 (compound 3b): The peracetylated generation 1 glucose dendrimer (1 g, 0.26 mmoles) was dissolved in anhydrous methanol and sodium methoxide was added to adjust the pH around 8.5-9. The reaction was stirred overnight at room temperature, then diluted with methanol and pH was adjusted with AMBERLIST® IR-120+ around 6-7. The reaction mixture was separated by filtration and the solvent removed by rotary evaporation, followed by water dialysis. Structure of D1-Glu6-OH₂₄ is shown below.

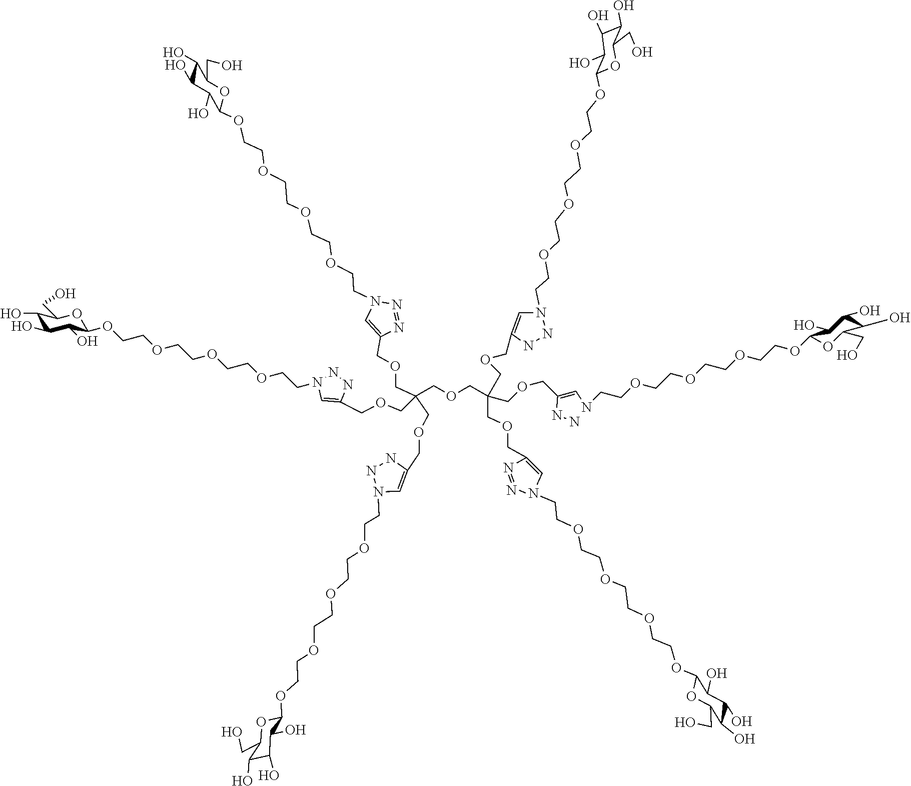

Synthesis of D1-Acetylene24 (compound 4): D1-GLu6-OH24 (2 g, 0.721 mmol) was dissolved in anhydrous dimethylformamide (DMF, 50 mL) by sonication. To this stirring solution, sodium hydride [60% dispersion in mineral oil](951 mg, 39.65 mmol) was slowly added in portions at 0° C. The solution was additionally stirred for 15 minutes at 0° C. This was followed by the addition of propargyl bromide (3.85 mL, 34.608 mmol, 80% w/w solution in toluene) at 0° C. and the stirring was continued at room temperature for another 6 h. The reaction mixture was quenched with ice and water, filtered and dialyzed against DMF, followed by the water dialysis to afford D1-acetylene24. Structure of D1-acetylene24 is shown below.

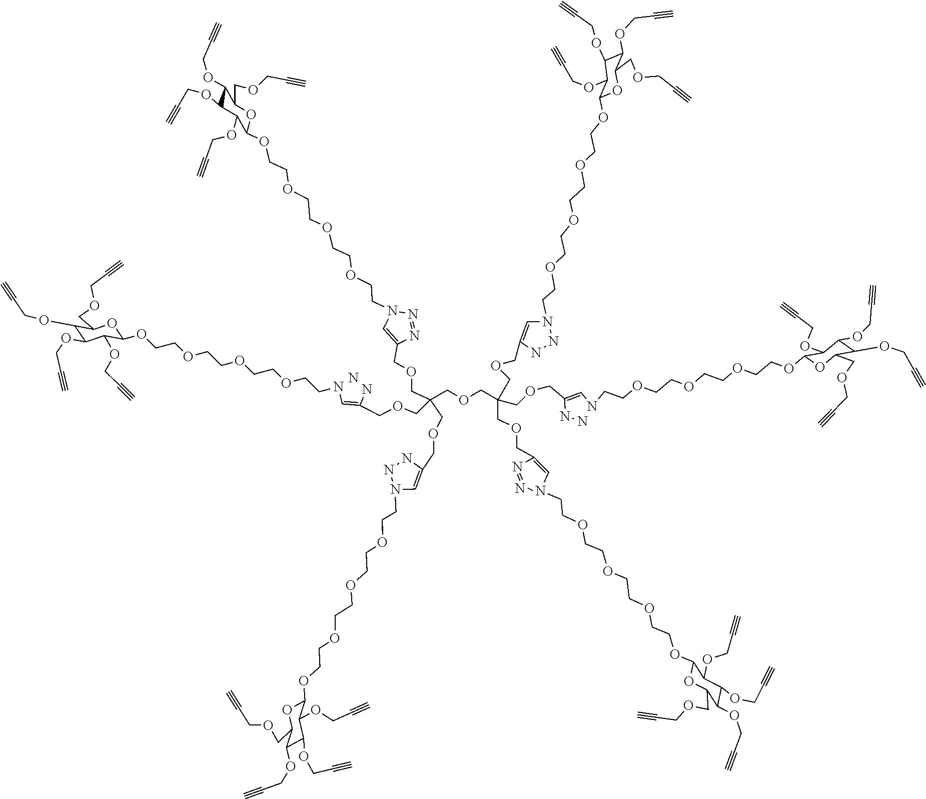

Synthesis of D2-Glu24-OAc96 (compound 5a): D1-acetylene dendrimer24 (0.5 g, 0.13 mmoles) and glucose-OAc-TEG-azide (2.2 g, 4 mmoles) were suspended in a 1:1 mixture of DMF and water in a 20 mL microwave vial equipped with a magnetic stir bar. To this CUSO₄·5H₂O (5 mol %/acetylene, 5 mg) and sodium ascorbate (5 mol %/acetylene, 10 mg) dissolved in the minimum amount of water were added. The reaction was irradiated in a microwave at 50° C. for 8 h. Upon completion, the reaction mixture was dialyzed against DMF followed by water dialysis containing EDTA. The EDTA was further removed by extensive water dialysis. The product was lyophilized to obtain D2-Glu24-OAc96.

Synthesis of D2-Glu24-OH96 (compound 5b): The peracetylated generation 2 glucose dendrimer D2-Glu24-OH96 was dissolved in anhydrous methanol and sodium methoxide was added to adjust the pH around 8.5-9.0. The reaction was stirred overnight at room temperature, then diluted with methanol and pH was adjusted with AMBERLIST® IR-120+ around 6-7. The reaction mixture was filtered to remove the resin and the filtrate was evaporated by rotary evaporation followed by water dialysis to obtain the product as off-white solid.

Synthesis of compound 6: D2-Glu24-OH96 (5b) (200 mg, 0.016 mmol) was dissolved in anhydrous dimethylformamide (DMF, 10 mL) by sonication. To this stirring solution, sodium hydride [60% dispersion in mineral oil](22 mg, 0.934 mmol) was slowly added in portions at 0° C. The solution was additionally stirred for 15 minutes at 0° C. This was followed by the addition of propargyl bromide (18.0 μL, 80% w/w solution in toluene) at 0° C. and the stirring was continued at room temperature for another 6 h. The solvent was evaporated using V10 evaporator system and the crude product was purified by passing through PD10 Sephadex G25 M column. The aqueous solution was lyophilized to afford the product as off-white solid.

Synthesis of Cy5-D2-Glu24-OH96 (compound 7): Compound 7 (200 mg, 0.016 mmol) and Cy5 azide (20.7 mg, 0.02 mmol) were suspended in a 1:1 mixture of DMF and water in a 25 mL round bottom flask equipped with a magnetic stir bar. To this CuSO₄·5H2O (5 mol %/acetylene, 0.3 mg) and sodium ascorbate (10 mol %/acetylene, 0.5 mg) dissolved in the minimum amount of water were added. The reaction was stirred at room temperature for 24 h. Upon completion, the DMF was evaporated using V10 and the purification was performed using PD10 SEPHADEX® G25 M column. The aqueous solution was lyophilized to afford the product as blue solid.

### Results

### Synthesis of Glucose Dendrimer

The synthesis of glucose dendrimer began with the construction of hexapropargylated core (1) and AB₄ peracetylated f-D glucose-PEG4-azide building blocks using Sharma, A. et al., Science Advances 2020, 6, (4), eaay8514; Sharma, R. et al., Chemical Communications 2014, 50(87), 13300-13303; and Sharma, R. et al., Polymer Chemistry 2015, 6(9), 1436-1444. To construct the dendrimer, the CuAAC click reaction was performed between the hexa-propargylated core (1) and the peracetylated β-Glucose-PEG₄-azide (2) using classical click reagents, a catalytic amount of copper sulfate pentahydrate and sodium ascorbate to achieve D1-GLU6-OAc24 (3a, FIG. 1A). The treatment of peracetylated dendrimer D1-GLU6-OAc24 (3a) under typical Zemplén conditions provided D1-GLU6-OH24 (3b). Complete acetate deprotection was confirmed by the absence of acetate protons in ¹H NMR spectrum. The terminal OH groups in G1 dendrimer 3b were then modified using NaH and propargyl bromide to obtain D1-Acetylene24 (4). The successful propargylation was confirmed by the appearance of alkyne peak corresponding at 62.4 ppm in NMR. The terminal alkyne groups in dendrimer 4 were the reacted with peracetylated D-glucose-PEG₄-azide (2) to produce D2-GLU24-OAc96 (5a). ¹H NMR clearly confirmed the formation of the product by the complete disappearance of 24 propargyl protons. The deprotection of acetate groups was carried out via Zemplén reaction to obtain the final dendrimer D2-GLU24-OH96 (5b). The disappearance of acetate peaks in the proton NMR confirmed the product formation. All the intermediates and final dendrimer were characterized via NMR, Mass and HPLC. The purity of D2-GLu24-OH96 is >99% based on HPLC analysis. The size and zeta potential of the final dendrimer was measured using dynamic light scattering. D2-GLU24-OH96 demonstrated a size of 4.36±0.38 nm with nearly neutral zeta potential of −6.19±0.56 mV. The physicochemical characterization of D2-GLU24-OH96 is presented in Table 1. The dendrimer D2-GLU24-OH96 is highly soluble in water/saline (>200 mg/mL).

TABLE 1
Physicochemical characterization of D2-GLU24-OH96

| | |
|---|---|
| Size | 4.4 nm |
| Zeta potential | −6.2 mV |
| No. of glucose | 24 (surface glucose) |
| HPLC purity | >99% |
| Molecular weight | 12.8 kDa |

Synthesis of fluorescently Labeled Glucose Dendrimer

To evaluate the targeting capability of D2-GLU24-OH96 via confocal microscopy and fluorescence spectroscopy, a near infra-red dye cyanine 5 (Cy5) waw attached on its surface. Two or three OH groups on the surface of D2-GLU24-OH96 (5b) were modified by reacting with propargyl bromide in the presence of sodium hydride to get compound 6 (FIG. 1B) which was further reacted with was reacted with Cy5-azide using CuAAC click reaction to obtain fluorescently labelled Cy5-D2-GLU24-OH96 (7).

The dendrimer D2-GLU24-OH96 is very stable in mouse and human plasma at physiological conditions for >72 h without showing any sign of degradation via HPLC. Moreover, the liver and kidney extracts from mice (intranasal) and rabbits (intravenous) administered with Cy5-D2-GLU24-OH96 showed intact dendrimer 4 h and 24 h post administration via HPLC, demonstrating that the dendrimer is not degrading in vivo and clearing intact.

### Example 2: Synthesis of Glucose Dendrimer-Drug Conjugates

### 1) Glucose Dendrimer-Drug Conjugates Using Copper Catalyzed Alkyne-Azide Click (CuAAC)

The synthesis of glucose dendrimer drug conjugates begins with the partial modification of OH groups to bring propargyl groups (FIG. 2A). On the other hand, the drugs are modified using releasable or non-releasable chemical linkages to bring an azide terminal groups through polyethylene glycol linkers. Few examples of glucose dendrimer-drug conjugates via CuAAC are described below.

### i) Synthesis of Glucose Dendrimer-Loperamide Conjugate

Procedure for synthesis of glucose dendrimer-loperamide conjugate (FIG. 2B): Loperamide hydrochloride was reacted with azido-PEG4-acid in the presence of DCC and DMAP in DCM at room temperature to afford loperamide-PEG-azide. On the other hand, partial modification of OH groups of the glucose dendrimer is carried out to bring approximately 10 propargyl groups, which are reacted with loperamide-PEG-azide to get glucose dendrimer-loperamide conjugate. The structure confirmation and the purity of the conjugate are achieved by the ¹H NMR and HPLC respectively.

### ii) Synthesis of Glucose Dendrimer-Rapamycin Conjugate

The synthesis of rapamycin-azide is achieved using Sharma, A. et al., Biomacromolecules 2020, 21, (12), 5148-5161.4 On the other hand, partial modification of OH groups of glucose dendrimer is carried out to bring ˜4 propargyl groups, which are reacted with rapamycin-azide to get glucose dendrimer-r conjugate (FIG. 2C). The structure confirmation and the purity of the conjugate are achieved by the ¹H NMR and HPLC, respectively.

iii) Synthesis of Glucose Dendrimer-Valproic Acid

This was achieved by first attaching an enzyme-sensitive clickable linker on VPA (FIG. 2D). The carboxylic acid group of VPA (compound 8 of FIG. 2D) was reacted with tetraethyleneglycol azide to obtain VPA-azide (compound 9 of FIG. 2D). On the other hand, dendrimer D2-GLU24-OH96 was partially modified by reacting approximately 7-8 hydroxyl groups with hexynoic acid in the presence of coupling agents DCC, DMAP to obtain an alkyne-terminating D2-Acetylene7 (10, FIG. 2D) which was further reacted with VPA-azide using Cu(I) catalyzed click (CuAAC) reaction in the presence of catalytic amount of CuSO₄·5H2O and sodium ascorbate to obtain D2-VPA (11, FIG. 2D) with on an average ˜7-8 molecules of VPA attached on the surface of dendrimer (FIG. 2D). The traces of copper were removed by dialyzing with ethylenediaminetetraacetic acid (EDTA). The final D2-VPA conjugate was thoroughly characterized by NMR and HPLC, and had an HPLC purity greater than 98%. The D2-VPA is highly stable at plasma conditions at pH (7.4) up to >48 hours, while the conjugate releases ˜15% of VPA in 1.5 hours, ˜33% in 48 hours at intracellular conditions (pH 5.5 plus esterases, Table 2) and the rest over a sustained period.

TABLE 2
In vitro drug release from glucose dendrimer
conjugates of D2-VPA

| | 4% Drug Release (37° C.) | |
|---|---|---|
| Time | pH 5.5 + Esterase | pH 7.4 |
| (Hours) | (intracellular condition) | (plasma condition) |
| 0 | <0.5% | <0.5% |
| 1.5 | ~17% | <0.5% |
| 4 | ~21% | <0.5% |
| 24 | ~24% | <0.5% |
| 48 | ~33% | <0.5% |

### 2) Glucose Dendrimer Conjugates Using a Combination of Click and Esterification/Amidation Reactions

The synthesis is achieved by partial modification of OH groups of glucose dendrimer is to bring propargyl groups, which are reacted with linker containing azide and amine termini to bring surface amine groups (FIG. 3A). On the other hand, the drug is modified using linker (hydrocarbon or PEG chains containing disulfide, ester, or amide linkages) with carboxylic acid, or —NHS ester terminal. The drug and dendrimer are the reacted using amidation reaction using coupling agents such as EDC and DMAP.

### i) Synthesis of Glucose Dendrimer-N Acetyl Cysteine (NAC) Conjugate:

The synthesis of glucose dendrimer NAC conjugate is achieved by the partial modification of OH groups of glucose dendrimers to bring propargyl groups, which are reacted with linker containing azide and amine termini to bring surface amine groups (FIG. 3B). On the other hand, the SPDP-NAC is obtained by the published procedure. NAC-SPDP and dendrimer are the reacted at pH 7.4 to obtain glucose dendrimer NAC conjugate.

### 3) Glucose Dendrimer Conjugates Using Copper-Free Biorthogonal Click Chemistry

The copper-free click reactions such as TCO-triazine (FIG. 4), strain promoted azide-alkyne, Staudinger ligation, DBCO-azide click reactions are used to attach drugs, siRNA, peptides, mRNAs, oligonucleotides, antibodies, and other biologics, where presence of copper and reducing agent can reduce the effect of the therapeutic, prophylactic or diagnostic agent.

### Example 3: In Vivo Brain Distribution of Glucose Dendrimer in a Mouse Seizure Model

Glucose dendrimers (GD) and PAMAM-GLU were further evaluated in a mouse seizure model to assess whether the specific colocalization with neurons extends to in vivo disease models. For this purpose, pilocarpine induced status epilepticus model was employed. 5 μl of either GD or PAMAM-GLU (40 μg/μl) was injected in right hemisphere of the brain using stereotaxic surgery. After the dendrimer delivery, mice were allowed to recover for 24 hours. Subsequently, pilocarpine was administered (300 mg/kg, i.p.) that resulted in behavioral seizures. Following 30 minutes of behavioral seizures, mice were euthanized, perfused, and fixed for histology. Immunofluorescent detection of nucleus (DAPI), neuron (Thy-1 YFP), microglia (IBA1 antibody) and GD/PAMAM-GLU (Cy5-conjugate) facilitated colocalization studies conducted on contralateral hemisphere to avoid effects of mechanical brain injury induced by stereotaxic injections. Both the GD and PAMAM-GLU were able to target neurons along with microglia in this seizure model. GD primarily localized in neuronal and microglial nucleus. However, in microglia significant levels of cytoplasmic GD is observed, unlike neurons. PAMAM-GLU is mainly distributed in the perinuclear cytoplasm of neurons and microglia.

These differences in GD and PAMAM-GLU distribution suggest that glucose dendrimer is a novel and unique nanocarrier with abilities to translocate into the nucleus and could be a promising platform for targeting nuclear processes.

### Example 4: In Vivo Distribution of Glucose Dendrimer in the Retina in a Mouse Model of Diabetic Retinopathy (DR)

Diabetes was induced in wildtype C57BL/6J mice with daily intraperitoneal injections of 60 mg/kg streptozotocin (STZ) for five consecutive days. Blood glucose levels were measured on day 3 after the final STZ injection, and 3 additional days of STZ treatment were performed if glucose levels were below 300 mg/dl. Animals were considered diabetic if blood glucose levels >350 mg/dL for 1 week. Four months (16-17 weeks) following induction of diabetes, 20 μg in 1 μl of the glucose dendrimer (or 1 μl of sterile saline as a control) was injected into the vitreous cavity of each mouse eye using a 30-gauge needle on a Hamilton syringe and the eyes were harvested 72 hours later. At the time of harvest, each retina was dissected from the eye and immediately processed for whole mount preparation to visualize en face the innermost retinal layer, comprised predominantly of neuronal cells, i.e., retinal ganglion cell (RGC) bodies and their axons which join to form the optic nerve. Immunofluorescence staining of the retinal whole mounts demonstrates the presence of the Cy5-labelled GD in Tuj-positive RGCs, indicating robust neuronal uptake. Moreover, the pattern of intracellular uptake clearly suggests the nuclear colocalization of GD in RGCs. The uptake of GD was also seen in microglia, labelled with Ibal, which mostly reside in the deeper synaptic layers of the retina but become activated in DR.

### Example 5: In Vivo Distribution of Glucose Dendrimer in the Retina in a Mouse Model of Oxygen Induced Retinopathy (OIR)

Neonatal C57BL/6J mice exposed to hyperoxia (75% 02) from day 7 to day 12 post birth were used in this study. Glucose dendrimer (5 ug/uL) was injected intravitreally on day 15 and on day 17 the mice were sacrificed, and the retinal tissues were obtained and fixed with 2% PFA for 24 hrs for immunohistochemistry. The retinal tissues were stained for Tuj-1 (1:500, abcam-ab18207) for ganglionic and bipolar cells and Iba-1 (1:500, Dako) for labelling microglia/macrophages for 12 hrs. at 4° C. For secondary antibodies anti-rabbit Cy3 and anti-rat 488 were used. The stained tissues were prepared for retinal flat mounts and imaged under confocal 710 microscope. Both 20× and 40× tile-Z stack images were obtained and processed using Zen software. The Z-stack images were processed using Imaris and 3D rendering images were constructed for demonstrating colocalization in microglia and ganglionic cells. Tiled-Z stack images confirm the glucose dendrimer demonstrate targeting and co-localization in both retinal neuronal (ganglionic and bipolar cells) and retinal microglia/macrophages which is different from our PAMAM dendrimers targeting and co-localizing in activated microglia/macrophages in this model. Hyperoxia exposure results in microglia activation and its distribution in all the layers of the retina whereas in normal retina, microglia are only found in outer retina.

### Example 6: Glucose Dendrimer is Taken Up by Glutamate Injured Neurons in Primary Neuronal Cell Cultures

Primary neuronal cultures were incubated with 10 μM glutamate and 10 g/ml dendrimer-Cy5 for 24 hrs. Subsequently, cultures were stained with anti-tubulin antibody (tuj1) to identify neurons. Confocal images were analyzed for the presence of Cy5-dendrimer.

Following 24 hours of treatment, generation 2 glucose dendrimer, Cy5-dendrimer, showed significant accumulation in neurons. Hydroxyl PAMAM-OH uptake was negligible in neurons in this in vitro glutamate-injury model.

### Example 7: Intracranial Administration of Glucose Dendrimer but not Hydroxyl PAMAM-OH Dendrimer Targets CA1 Hippocampal Pyramidal Neurons in the Pilocarpine Model of Seizures in Mice

Intracranial injection of PAMAM-OH-Cy5 or GD2-Cy5 (4 μl of 50 μg/μl) was followed by seizure induction with 300 mg/kg of pilocarpine IP after 24 hours. 30 mins after seizure induction (Racine scale 3 seizure), the mice were sacrificed, and brains perfused for immunohistochemistry. Generation 2 glucose dendrimer (GD2) showed significant uptake in contralateral CA1 neurons. On the other hand, PAMAM-OH uptake was negligible in the contralateral CA1 neurons, but mostly showed diffused microglial uptake in the ipsilateral side.

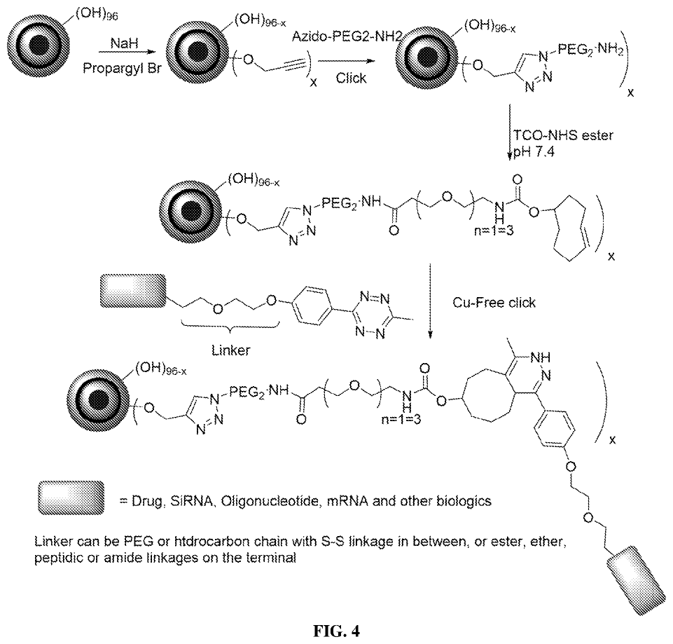

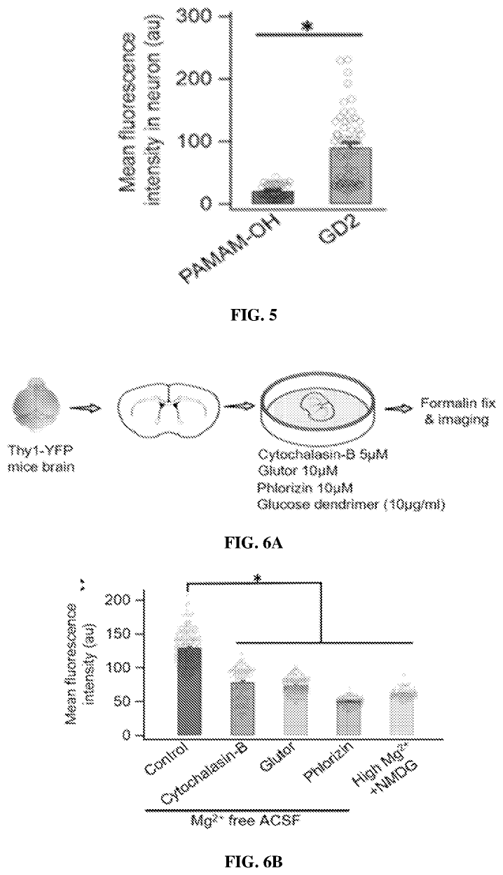

The neuronal fluorescence in the contralateral side CA1 neurons was >100-fold higher than that of hydroxyl PAMAM dendrimer (FIG. 5). In healthy animals (control mice without seizure induction), the contra-lateral CA1 neurons did not show GD2 colocalization indicating no neuronal uptake in these neurons.

### Example 8: GD2 Uptake is Dependent on Neuronal Activity and GLUT Transporter

### Methods

300 μm cortical brain sections were pre-treated for 30 minutes with Mg₂+ free artificial cerebrospinal fluid (ACSF) (increases neuronal firing) or ACSF containing Mg₂+/NMDG (suppressed neuronal activity) or Mg2+ free ACSF with cytochalasin B (5 μM) or glutor (10 μM) or phlorizin (10 μM). After pre-treatment, brain sections were incubated with GD2-Cy5 (10 μg/ml) for 30 minutes followed by formalin fixation and confocal imaging. Mean fluorescence intensities for GD-Cy5 were evaluated in YFP-expressing cortical neurons. Confocal images of fixed sections showing DAPI (nucleus), YFP (neurons) and GD2-Cy5 with different treatments were taken and analyzed.

### Results

Higher metabolic activity by injured/hyperactive neurons can increase glucose requirement and thus can increase generation 2 glucose dendrimer (GD2) uptake. Accordingly, it was hypothesized that suppression of neuronal activity will inhibit GD2 dendrimer uptake. Indeed, it was observed that a significant decrease in GD2 colocalization in neurons when incubated with buffer solution containing N-Methyl-D-glucamine (instead of NaCl) and high MgCl₂ (5 mM) known to suppress neuronal activity (Dribben, W. H. et al., Cell Death & Disease 2010, 1 (8), e63-e63; Stanojevic, M. et al., Journal of Elementology 2016, 21 (1), 221-230) (FIGS. 6A and 6B). Furthermore, blocking glucose transporters (GLUT) using two different pharmacological antagonists: cytochalasin B (non-specific GLUT inhibition) and glutor (GLUT 1-3 inhibitor) or blocking SGLT 1 and 2 by Phlorizin diminished GD2 uptake by neurons, suggesting involvement of glucose transporters in uptake (FIG. 6B).

### Example 9: GD2 Targets Select Neurons in Acute Brain Slices Ex Vivo in a Rabbit Model of Cerebral Palsy

Acute hippocampal brain sections from newborn rabbits with cerebral palsy when incubated with GD2-Cy5 (20 μg/ml) for 45 mins in artificial cerebrospinal fluid (ACSF) took up GD2 as evidenced by confocal images (n=3 rabbit kits). The confocal images show the CA1 pyramidal neuron layer with GD2-Cy5 accumulation in select neurons (stained with PGP). This model demonstrates delayed neuronal injury following intrauterine endotoxin insult (Balakrishnan, B. et al., Developmental neuroscience 2013, (5), 396-405; and Kannan, S. et al., Sci Transl Med 2012, 4 (130), 130ra46-130ra46). A similar uptake in neurons is seen in vivo in a juvenile rabbit model of controlled cortical impact induced traumatic brain injury. This suggests that the neuronal targeting occurs irrespective of the mechanism of injury and the species.

### Example 10: GD2 Localizes in Neurons Upon Intranasal Delivery in a Mouse Model of Pilocarpine Induced Seizures

Generation 2 glucose dendrimer labeled with Cy5 (GD2-Cy5) was administered intranasally (100 μg in 10 μl) following IP injection of 300 mg/kg of pilocarpine. Mice dosed with GD2-Cy5 were perfused and fixed after 4 hours. Confocal images show Cy5 intensities localized in neuronal layer in olfactory bulb, cortex and hippocampal CA1 regions, known to be affected by pilocarpine. This indicates that intranasal administration is a viable option for delivery of glucose dendrimers to the brain.

### Example 11: GD2-VPA Conjugate Treatment Reduces Seizure Severity Induced by Pilocarpine Injection

Seizure was induced using pilocarpine. After visual verification of active seizures (Racine scale 3 and above), 100 μg of GD2-VPA (in 101 of saline which contains ˜0.3 mg/kg of VPA) was administered intra-nasally (15 minutes after pilocarpine injection). 1 μl of saline or GD2-VPA was administered in each nostril every 2 minutes. GD2-VPA treated mice showed faster recovery after seizure induction. The characteristic posture loss and tail stiffening in seizure was comparably lower in GD2-VPA treated mice compared to saline-administered mice. Mice treated with GD2-VPA had better mobility and activity sooner than the saline treated animals. When analyzed 2 hours post-intranasal drug administration, GD2-VPA treated mice displayed minimal splaying and tail stiffness. Increased motility was seen for the GD2-VPA mice at 1 hour post induction of surgery when compared to saline and VPA treated mice.

In pilot studies, intranasal GD2-VPA treatment decreases frequency of spike-wave discharges acutely and prevents ictal events after pilocarpine administration. Wireless electroencephalogram (EEG) recording devices were implanted in adult mice (25-30 g) and were allowed to recover over 3 days. Seizures were induced with pilocarpine while EEG were being recorded. Five minutes of EEG recordings immediately before and 15 minutes after intra-nasal saline or GD2-VPA administration were analyzed for electrographic events. Spike wave discharges increased in count and mean amplitude in the saline-treated animals while both the count and mean amplitude of spike-wave depolarizations decreased in mice treated with intranasal GD2-VPA. Mice were administered a second dose of pilocarpine after 24 hours and EEG was recorded for 3 hours after. Saline-treated mice showed ictal events with high frequency spiking for more than >150 seconds while GD2-VPA treated animals did not develop any ictal events. This indicates that a single dose of GD2-VPA demonstrates a sustained effect even after 24 hours.

In summary, these data demonstrate that glucose dendrimers localize primarily in neurons and the uptake appears to be mediated by glucose transporter(s). Moreover, treatment with intranasal GD2-VPA led to improvement in seizure frequency and mobility in the acute phase, indicating that this is a powerful platform to deliver drugs specifically to the neurons. Experimental data have shown that glucose dendrimers are primarily taken up by injured neurons unlike hydroxyl PAMAM dendrimers that target ‘activated’ microglia. Previously published work has shown in multiple models that hydroxyl PAMAM-OH dendrimers do not target neurons, but primarily target only activated microglia/macrophages in the injured area (Iezzi, R. et al., Biomaterials 2012, 33 (3), 979-988; Kambhampati, S. P. et al., Investigative ophthalmology & visual science 2015, 56 (8), 4413-4424; Kannan, S. et al., Science Translational Medicine 2012, 4 (130), 130ra46; Khoury, E. S. et al., Theranostics 2020, 10 (13), 5736-5748; Liaw, K. et al., Bioengineering & translational medicine 2021, 6 (2), e10205; Mishra, M. K. et al., ACS nano 2014, 8 (3), 2134-2147; Sharma, A. et al., Biomacromolecules 2020, 21 (9), 3909-3922; Sharma, A. et al., Theranostics 2018, 8 (20), 5529-5547; Sharma, R. et al., Journal of Controlled Release 2020, 323, 361-375). The hydroxyl PAMAM dendrimer is used as a ‘control’ to establish the differential cellular targeting of the two types of dendrimers.

When administered intranasally, both glucose dendrimers and hydroxyl PAMAM dendrimers would be transported to the brain, consistent with prior findings. However, in the healthy brain there is no uptake or retention at 8-24 hour period. This suggests that affinity of the glucose dendrimer to GLUT/SGLT in healthy brain cells is not strong enough compared to its rapid diffusion rate to enable uptake. In contrast, in the presence of seizure induced activity, cerebral palsy, or traumatic brain injury, the increased activity of glucose transporters on injured neurons enables the specific uptake and retention. This is unlikely to be a size or surface hydroxyl effect since hydroxyl PAMAM dendrimers do not show uptake in neurons or microglia in seizing animals. Increased ‘non-specific endocytosis’ of activated neurons is also unlikely to be the reason for uptake, since hydroxyl PAMAM would have also shown uptake if that was the case. The presence of surface glucose on GD is important.

### Example 12: Glucose Dendrimers for Targeted Drug Delivery to Hyperactive Neurons

### Materials and Methods

### GD2 and Sodium-Valproate (VPA).

### Release Study of GD2-VPA Conjugates Under Intracellular Conditions

pH 7.4: stable conjugate with no indication of VPA release up to 24 h. Intracellular conditions: Fast release with ˜15% release in 1-2 h and 25% in 24 h.

### Mice

Thy1-YFP and wild type C57BL/6 mice were purchased from Jackson Laboratoeis and were subsequently bred and housed in the animal facility with a 12-h light and 12-h dark cycle. Thy1-YFP mice were used for in vivo dendrimer localization studies according to the approved protocol from the Johns Hopkins University Animal Care and Use Committee (IACUC). Thy1-YFP mice were also used for acute brain slice experiments. Wild type C57BL/6 mice were used for pilocarpine-induced seizure studies as described by Arshad, Bio-protocol 10 (2020). Scopolamine methyl nitrate was injected intraperitoneally (2 mg/kg, Sigma-Aldrich S2250), followed by pilocarpine hydrochloride (i.p.; Sigma-Aldrich P6503 at 300 mg/kg) after 30 minutes. Behavioral seizures were monitored and scored using modified Racine's scale (M. N. Arshad, J. R. Naegele, Bio-protocol 10 (2020)).

Isolation and Culture of Primary Neurons from Rabbit Kit Brain Tissue

Primary hippocampal neuron cultures were used in this study using standard procedure. Briefly, brain hippocampi were micro dissected from postnatal day 1 rabbit kit followed by removing blood vessels and meninges in ice-cold dissection solution containing 1× Hank's Balanced Salt solution, 1× penicillin/streptomycin, 1 mM sodium pyruvate, 10 mM HEPES, and 30 mM glucose. Subsequently, hippocampi were chopped and digested using the Papain Dissociation kit according to the manufacturer's protocol (Worthington, USA). The digested tissue in the buffer was triturated with a sterile fire-polished glass pipette to dissociate tissue clumps and cells and then centrifuged at 4° C. for 5 minutes at 300 g. The pellet was resuspended in Earle's Balanced Salt Solution containing ovomucoid protease inhibitor with bovine serum albumin and deoxyribonuclease. Next, a discontinuous density gradient was prepared by pipetting the cell suspension onto a 5-ml layer of albumin-ovomucoid inhibitor solution and centrifuged at 70 g for 6 minutes at room temperature to remove the supernatant containing noncellular debris. The resulting cell pellet was resuspended in neurobasal medium supplemented with 1× GlutaMAX, 2% B27, 1% penicillin-streptomycin, and 1% heat-inactivated horse serum. 50,000 cells were seeded on poly-D-lysine- and laminin-coated coverslips and incubated at 37° C. After 24 hours, media was replaced with fresh media containing 5 μM cytosine arabinoside. Half of the culture medium was changed every week.

For immunocytochemistry, neuron cultures were washed with PBS and fixed with formalin for 10 min followed by another PBS wash. Next, the cultures were blocked with 10% donkey serum for 30 minutes and subsequently incubated overnight with anti-beta III tubulin antibody (1:1000, Abcam, MA. USA). Upon washing the culture coverslips, they were incubated with alexa fluor conjugated secondary antibody (donkey anti-rabbit AF488, 1:250) for 1 hour. The coverslips were then treated with DAPI (1:5000) for 5 min and washed and mounted.

### Animal Model of Cerebral Palsy

Time pregnant New Zealand white rabbits (Robinson Services Inc) underwent laparotomy surgery at gestational day 28 according to the protocol approved by the Johns Hopkins University Animal Care and Use Committee (IACUC). Briefly, during laparotomy, rabbits received a total of 1800 EU (endotoxin units) of LPS (Escherichia coli serotype 0127:B8, Sigma-Aldrich, St. Louis, MO) injection along the wall of the uterus (S. Kannan et al., Science translational medicine vol. 4, 130ra146 (2012)). Rabbits were induced on G30 with intravenous injection of Pitocin (0.5 U/kg) (JHP Pharmaceuticals, Rochester, MI) and sacrificed for live brain slice experiments on day of birth.

### Behavioral Seizures

For efficacy studies, a single dose of pilocarpine 300 mg/Kg i.p. was used induce the status epilepticus (SE). Prior to seizure induction, scopolamine methyl nitrate was injected intraperitoneally (2 mg/kg, Sigma-Aldrich S2250) to antagonize the peripheral effects of pilocarpine. The animals were video-recorded in their the individual cages and the convulsive activity was scored based on the modified Racine scale (K. Borges et al., Experimental neurology 182, 21-34 (2003); C. J. MUller, et al, Experimental neurology vol. 219, 284-297 (2009)) as follows:

Stage 0: Normal activity,

Stage 1: freezing and slight head nodding,

Stage 3: Head bobbing, wet dog shakes, straub tail,

Stage 4: partial myoclonus, occasional jerks, body tremors, intensified freezing or uncontrolled circling movement,

Stage 4: Increased immobility and freezing, uncontrolled circling movement,

Stage 5: Continuous straub tail, loss of limb control followed by generalized tonic clonic seizures, oro-alimentary automatism or one episode of rearing,

Stage 6: loss of balance, more than one episode of rearing followed by occasional falling, jumping and rolling over, generalized tonic extension of the body, cardiopulmonary collapse and death. The continuous behavioral seizures were scored for 180 minutes.

Stage 1 and 2 were categorized as low grade, stage 3 as medium grade and stage 5-6 as high-grade seizures. Each episode counted at least lasted for 30 s, typically separated by the other with variable durations. Periods between stage 3-6 were considered as continuous low-grade seizures as observed in these animals for 180 minutes. Repeated and sufficiently prolonged continuous seizures of medium grade or higher with short intervals (at least 1 such seizure within 5 minutes) was considered as the development of status epilepticus (H. Shibley, B. N. Smith, Epilepsy research 49, 109-120 (2002); E. Trinka et al., Epilepsia 56, 1515-1523 (2015)). Behavioral seizures were monitored and scored using modified Racine's scale (M. N. Arshad, J. R. Naegele, Bio-protocol 10 (2020)).

### Ex Vivo Brain Slices Preparation

CP rabbit kits or Thy1-YFP mice were deeply anesthetized with isoflurane and decapitated. Brains were removed and transferred to oxygenated (95% O₂/5% CO₂), ice-cold N-Methyl-D-glucamin (NMDG)-based buffer (Buffer1, in mM: 92 NMDG, 2.5 KCl, 10 MgSO₄, 0.5 CaCl₂, 1.2 NaH₂PO₄, 30 NaHCO₃, 25 glucose, 20 HEPES, 5 sodium ascorbate, 3 sodium pyruvate, 2 thiourea; pH 7.4). Coronal brain slices (300 μM) were then obtained using a vibratome (VT1200, Leica). Slices were first incubated in the NMDG-based solution at 34° C. for 10 minutes, then transferred and maintained in the same solution for an hour at room temperature before starting subsequent experiment. Dendrimer-Cy5 was added in 5 ml of Buffer2 (in mM: 125 NaCl, 2.5 KCl, 1 MgCl₂, 2 CaCl₂, 1.25 NaH₂PO₄, 26 NaHCO₃, 20 glucose; pH 7.4) containing brain slices and incubated for 30 min while oxygenation at room temperature. Brain slices were fixed with formalin and immune-stained for neuronal (MAP2 or PGP) proteins for visualized under an upright microscope equipped with laser scanning confocal optics (LSM 880, Zeiss).

### Immunohistochemistry

For immunohistochemistry, animals were transcardially perfused with saline and post-fixed in 10% formalin for 24 hours followed by cryoprotection in 30% sucrose solution for 2 days. Coronal sections (30 μm, 1:6 series) were collected and blocked by 5% normal donkey serum in 0.1 M PBS. Subsequently, sections were then incubated overnight at 4° C. with goat anti-IBA1 (1:500, Abcam, MA. USA), followed by incubation with secondary antibodies for 2 hours at room temperature. Upon incubation with DAPI (1:1000, Invitrogen) for 15 minutes, slides were washed, dried and cover slipped with mounting medium. Similarly, for acute CP brain slice immunostaining, slices were fixed overnight, washed and incubated with primary antibody anti-PGP antibody (1:100, Abcam, MA. USA) followed by 2 hour incubation in secondary antibody solution. Confocal images were acquired with Zeiss ZEN LSM 710 (Zeiss, CA, USA) and processed with ZEN software.

### Statistics

The data was summarized using mean values ±S.E.M and used either t-test or one way-ANOVA for group comparisons. As appropriate, the Bonferroni corrections were used to adjust for multiple comparisons. The analyses were performed using GraphPad Prism software. Statistical significance was set as P<0.05, and all tests were two-sided.

### Results

### Synthesis and Characterization of Glucose Dendrimer (GD)

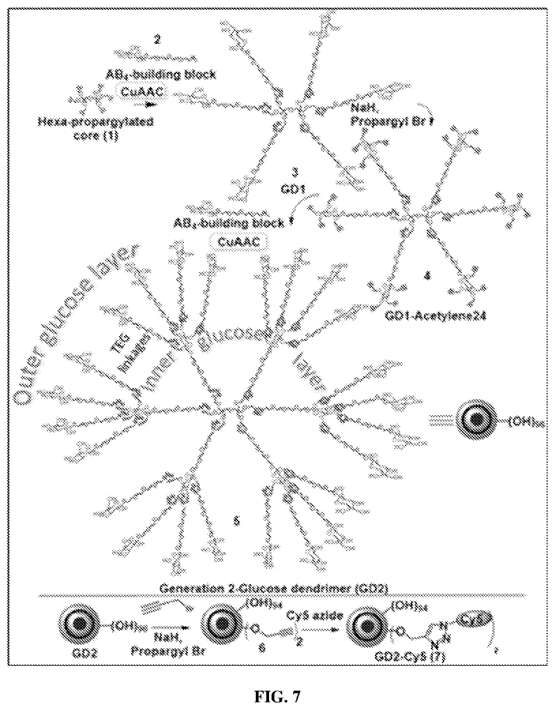

A synthetic plan was designed and standardized (FIG. 7) for the preparation of generation 2 glucose dendrimer (GD2) using a highly efficient click chemistry approach. Approximately 1 g quantities of this glucose dendrimer platform were validated. The synthesis of GD2 was achieved in an expedited manner and began by reacting hexapropargylated core (1) with AB₄ Q-D-glucose-PEG4-azide building block (2) via click reaction to obtain generation 1 glucose dendrimer (GD1, 3, FIG. 7). The hexapropargylated core (1) and AB₄ β-D-glucose-PEG4-azide (2) building block was synthesized using protocols by Sharna et al., Sci Adv 6, eaay8514 (2020); A. Sharma et al., Biomacromolecules 21, 5148-5161 (2020); R. Sharma et al., Biomacromolecules 22, 3574-3589 (2021)). The hydroxyl groups on GD1 were further propargylated to obtain GD1-Acetylene24 (4), which was again reacted with AB₄ β-D-glucose-PEG4-azide (2) to obtain generation 2 glucose dendrimer (GD2, 5) with 24 glucose molecules containing 96 surface hydroxyl groups. A near infrared fluorescent tag Cy5 was attached on GD2 by propargylating ˜2-3 hydroxyl groups to bring alkyne containing dendrimer (6) which was further reacted with Cy5-azide to obtain fluorescently labeled GD2-Cy5 (7). The dendrimers were purified using tangential flow filtration technique leading to highly pure products. The final dendrimer and intermediates were characterized using 1H & 13C NMR for structure, and HPLC for purity. The physicochemical characterization of GD2 is presented in Table 1. The HPLC purity of the final dendrimers was >99%. The GD2 is highly soluble in water/saline (>200 mg/mL). The GD2 is very stable in mouse and human plasma at physiological conditions for 72 hours without showing any sign of degradation via HPLC.

The GD2 demonstrated high stability in mouse and human plasma at physiological conditions for at least 72 hours without showing any signs of degradation (HPLC). Moreover, the liver and kidney extract from mice (intranasal) and rabbits (intravenous) administered with GD2 showed intact dendrimer 4 hours and 24 hours post administration via HPLC, demonstrating that the dendrimer is not degrading in vivo and clearing intact.

### Neuronal Uptake of Glucose Dendrimer (GD2) in In-Vitro Primary Culture and Ex-Vivo Brain Slices

Glutamate excitotoxicity is ubiquitous across many pathological brain conditions. To mimic similar injury conditions, primary rabbit neuron cells were cultured and exposed to 10 μM glutamate for 24 hour that led to excitotoxicity of neurons. Concomitant exposure to 10 μg/ml dendrimer for 24 hours showed significant accumulation of GD2 in neurons. Sister neuronal cultures exposed to 10 μM glutamate and 10 μg/ml PAMAM-OH had negligible accumulation of PAMAM-OH dendrimer in neurons in this in-vitro glutamate-injury model. Data demonstrates intra-neuronal uptake of GD2 in in vitro glutamate injury model. Briefly, 3-4 weeks old primary neuronal cultures were incubated with 10 μM glutamate and 10 μg/ml dendrimer-Cy5 for 24 hours. Subsequently, the cultures were immuno-stained with anti-tubulin antibody to identify neurons. Confocal images were acquired and analyzed for the presence of dendrimer in Cy5-channel (633-666 nm). The data indicated that GD2 localized in pyramidal neurons whereas PAMAM-OH did not.

The unique neuronal targeting propensity of GD2 in ex vivo acute brain slices from new born rabbits with brain injury caused by maternal systemic LPS-induced inflammation (rabbit model of cerebral palsy). This ex vivo brain slice model provides unique advantages over primary neuronal culture as it largely preserves the neuronal intrinsic and synaptic architecture. Acute hippocampal brain sections from newborn rabbits with cerebral palsy, when incubated with GD2-Cy5 (20 μg/ml) for 45 minutes. The treated acute brain sections were formalin-fixed (10% formalin) and immune-stained for neuronal markers: ubiquitin carboxy-terminal hydrolase L1 (PGP) or MAP2. Confocal microscopy images showed abundance of GD2-Cy5 in hippocampal pyramidal neurons (n=3 rabbit kits) (data not shown). Significant levels of GD2-Cy5 were observed in CA1 pyramidal cell layer when hippocampal sections were incubated with GD2-Cy5 (20 μg/ml) for 45 minutes in standard artificial cerebrospinal fluid (ACSF). These data demonstrate that GD2 targets select CA1 neurons in acute hippocampal brain slices collected from rabbit model of cerebral palsy.

### In Vivo GD2-Cy5 Uptake in Neurons Depends on Neuronal Hyperactivity.

Intracranial injection of PAMAM-OH or GD2 conjugated to Cy5 (4 μl of 50 μg/μl) followed by seizure induction with 300 mg/kg of pilocarpine IP after 24 hours, facilitated GD2 uptake by contralateral CA1 neurons. PAMAM-OH uptake was negligible in the contralateral CA1 neurons. 30 minutes after seizure induction (Racine scale 3 seizure), the mice were sacrificed and brains perfused for immunohistochemistry. These data demonstrate the feasibility of specific delivery of drugs by the GD2-dendrimer platform to hyperactive neurons.

### Glucose Transporters Mediates GD2-Cy5 Uptake

Higher metabolic activity by injured/hyperactive neurons can increase glucose requirement and thus can increase GD2 uptake. It was hypothesized that suppression of neuronal activity will inhibit GD2 dendrimer uptake. A significant decrease in GD2 colocalization in neurons was observed when they were incubated with buffer solution containing N-Methyl-D-glucamine (instead of NaCl) and high MgCl₂ (5 mM) known to suppress neuronal activity. Blocking glucose transporters (GLUT) using two different pharmacological antagonists, cytochalasin B (non-specific GLUT inhibition) and glutor (GLUT 1-3 inhibitor), diminished GD2 uptake by neurons, indicating involvement of GLUT-dependent uptake.

### GD2 Localizes in Neurons Upon Intra-Nasal Delivery in a Mouse Model of Pilocarpine Induced Seizures

Glucose dendrimer labeled with Cy5 (GD2-Cy5) was administered intranasally (100 μg in 10 μl) following IP injection of 300 mg/kg of pilocarpine. Mice dosed with GD2-Cy5 was perfused and fixed after 4 hours. Confocal images show Cy5 intensities localized in neuronal layer both in olfactory bulb, cortex and hippocampal CA1 region. These data indicate that GD2-Cy5 localizes in olfactory bulb and CA1 neuronal cell layer upon intra-nasal administration. This indicates that intranasal administration is a viable option for delivery of GD2 to the brain.

### Synthesis and Validation of GD2-VPA Conjugate

Motivated by neuronal targeting by GD2-Cy5, valproate (VPA) was conjugated to the GD2 dendrimer (FIG. 7). The synthesis of GD2-VPA was achieved by first attaching an enzyme-sensitive clickable linker on VPA (FIG. 7). The carboxylic acid group of VPA (8) was reacted with tetraethyleneglycol azide to obtain VPA-azide (9). On the other hand, GD2 was partially modified by reacting by about 7-8 hydroxyl groups with hexynoic acid in the presence of coupling agents DCC, DMAP to obtain an alkyne-terminating GD2-Acetylene7 (10) which was further reacted with VPA-azide using Cu(1) catalyzed click (CuAAC) reaction in the presence of catalytic amount of CuSO₄·5H₂O and sodium ascorbate to obtain GD2-VPA (11) with on an average ˜7-8 molecules of VPA attached on the surface of dendrimer (FIG. 7). The click chemistry makes the synthesis facile and robust thereby providing good control on synthesis and ligand loading. The traces of copper were removed by dialyzing with ethylenediaminetetraacetic acid (EDTA). The final GD2-VPA conjugate was thoroughly characterized by NMR and HPLC, and had an HPLC purity greater than 98%. The GD2-VPA is highly stable at plasma conditions at pH (7.4), while the conjugate releases ˜15% of VPA in 1.5 hours and ˜25% in 24 hours at intracellular conditions (pH 5.5 plus esterases, FIG. 7).

### GD2-VPA Protects Against Pilocarpine-Induced Behavioral Seizures

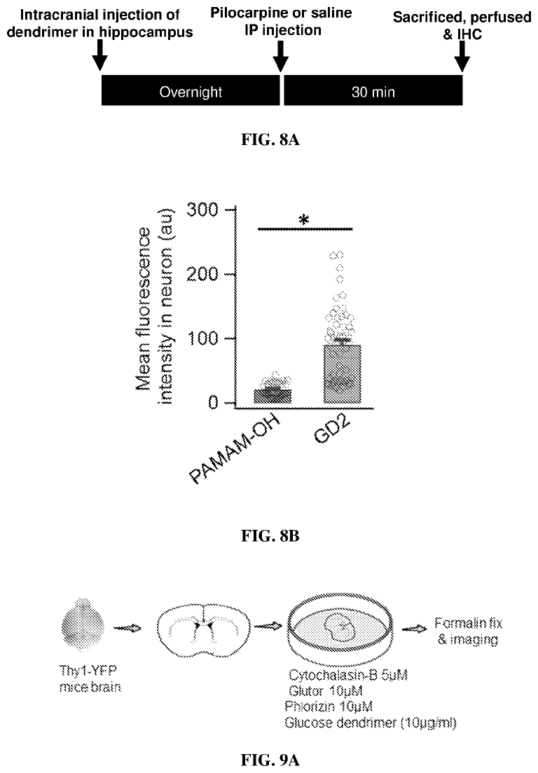

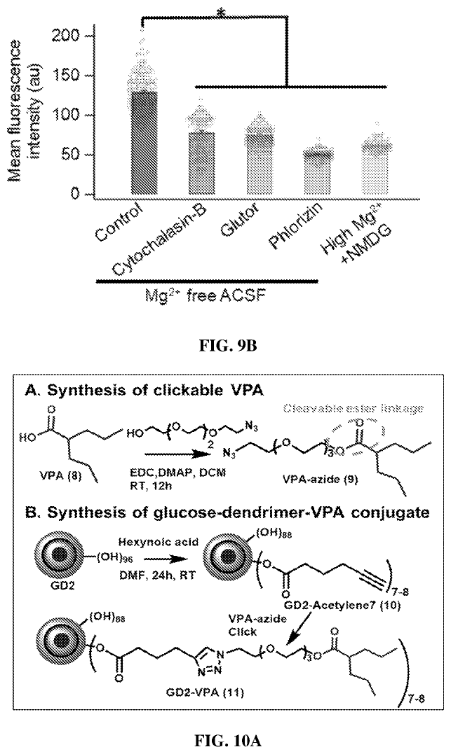

To evaluate the efficacy of GD2-VPA, pilocarpine mouse model of status epilepticus was used. Pilocarpine is a potent muscarinic agonist that can generate sequential behavioral and electrographic seizures. Upon pilocarpine IP injection (300 mg/kg) and followed by visual verification of active seizure (Racine scale 3 and above), GD2-VPA (0.3 mg/kg of VPA basis) was administered intra-nasally (15 minutes after pilocarpine injection). 1-2 μl of saline or GD-VPA solution (10 μg/μl) was administered in each nostril every 2 minutes. Saline treated mice showed increased splaying and tail stiffness compared to GD2 VPA after 1 hour of pilocarpine. Additionally, exploratory behavior was significantly increased with GD2-VPA treatment (FIG. 8C). GD2-VPA prolonged the latency to first episode of both medium and high-grade seizures and reduced the total duration of high-grade seizures post pilocarpine administration.

### Uptake of GD2-Cy5 Dendrimer by Select Neurons (Syngap Mouse Seizure Model)

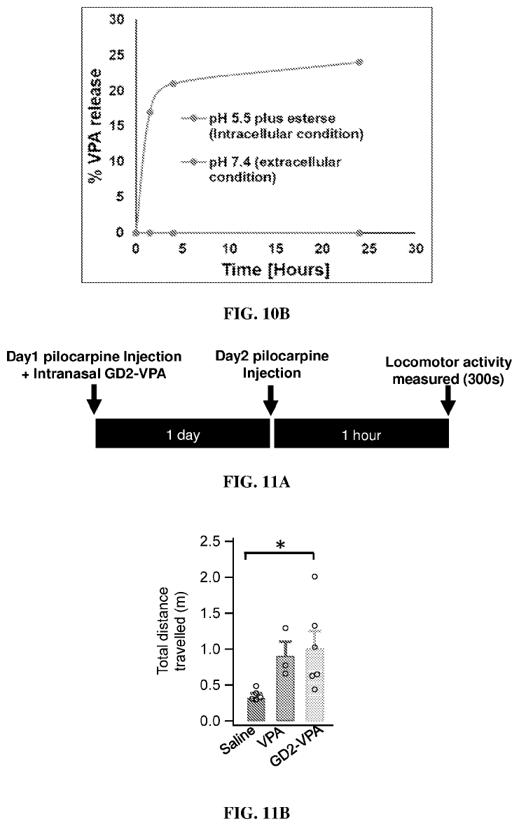

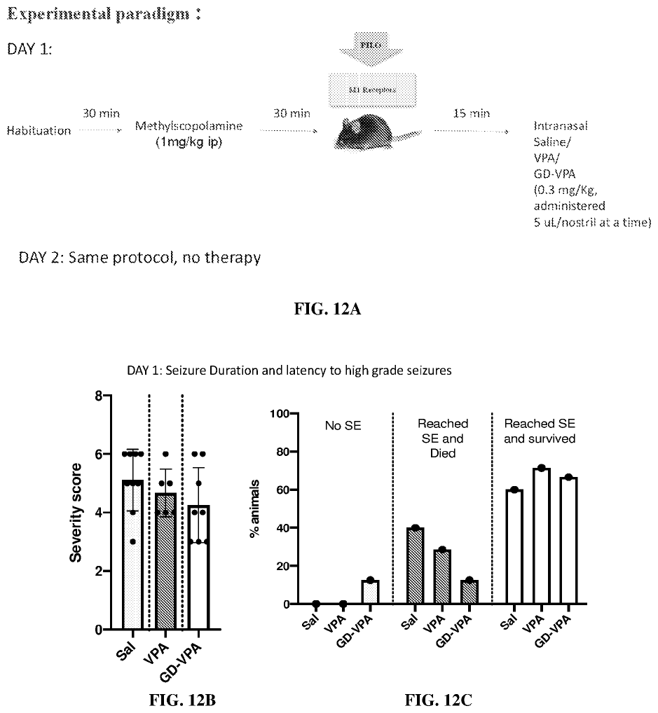

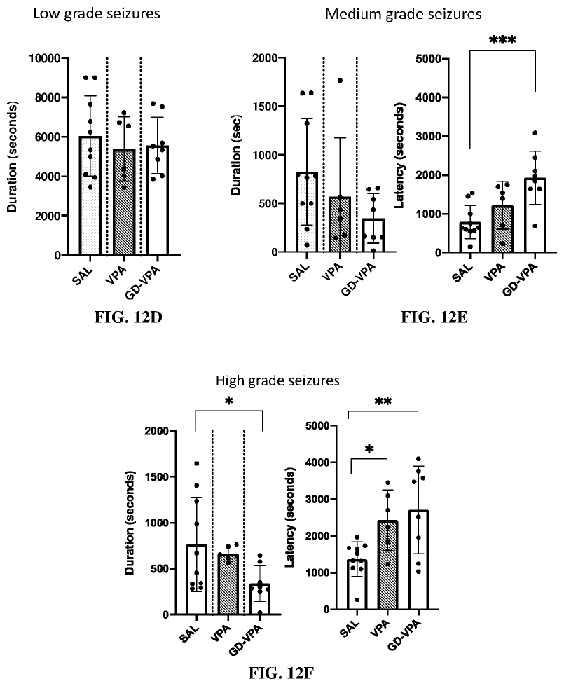

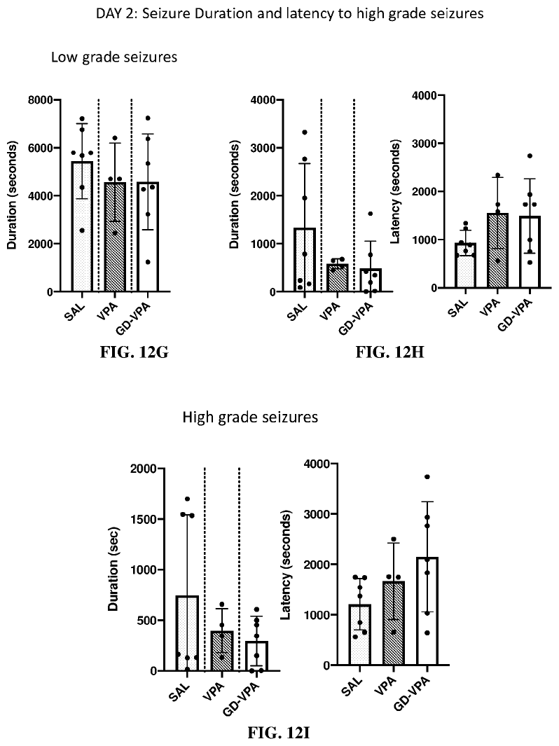

Experiments were performed as demonstrated in the experimental timeline of FIG. 12A. A modified Racine scale was used as shown in Table 3 below. Category 3-5 seizures were typically of 15-90 s duration and were separated by periods of relative inactivity or other stages of variable duration. The periods between category 3-6 seizure events were usually marked by continuous category 1- and 2-type seizure activity. A mouse that experienced a minimum of three category 3-6 seizure events within 2 h following pilocarpine injection was considered to have undergone SE (Shibley et al, 2002).

TABLE 3
Modified Racine Scale (Racine 1972; Borges, 2002)

| Stage | Behavior |
|---|---|
| 1 | freezing |
| | slight head nodding |
| 2 | Head bobbing and shakes |
| | tail arching |
| | wet dog shakes |
| | extended or arched body |
| 3 | partial myoclonus-occassional jerks |
| | body tremors |
| | intensified freezing or uncontrolled |
| | circling movement |
| 4 | intensified immobility, flexed arms |
| | or continous limb clonus |
| | Myoclonic jerks |
| | continous straub tail |
| 5 | loss of limb control |
| | generalized tonic clonic seizures |
| | oro-alimentary automatism |
| | 1 episode of rearing |
| 6 | loss of balance |
| | >1 rearing episodes and occasional falling |
| | jumping, rolling over |
| | generalized tonic extension of body |
| | cardiopulmonary collapse-death |

### Results

The results are shown in FIGS. 12B3-12I. FIGS. 12B-12I show that uptake of GD2-Cy5 dendrimer by select neurons (Syngap mouse seizure model). FIGS. 12B and 12C are bar graphs of the scores for seizure duration and latency to high grade seizures on day 1. FIGS. 12D-12F are bar graphs showing the seizure duration scores for low grade seizures (FIG. 12D), medium grade seizures (FIG. 12E), and high grade seizures (FIG. 12F). FIGS. 12G-12I are bar graphs showing the day 2 seizure duration and latency to high grade seizures for low grade seizures (FIG. 12G), medium grade seizures (FIG. 12H), and high grade seizures (FIG. 12I).

Using different models of increasing complexity, it was demonstrated that GD2-Cy5 internalizes in neurons under hyperactive conditions. Pharmacological blocker experiments suggest that glucose dendrimers localize primarily in neurons and the uptake appears to be mediated by glucose transporter. Moreover, treatment with intranasal GD2-VPA led to improvement in seizure frequency and mobility in the acute phase, indicating that this is a powerful platform to deliver drugs specifically to the neurons.

Neurons maintain negative membrane potential at resting state and transiently depolarizes and repolarizes during an active action potential (Alle, et al., Science 325, 1405-1408 (2009)). Transient membrane potential fluctuations also occur during sub-threshold synaptic neuro-transmission (Harris, et al., Neuron 75, 762-777 (2012)). Preserving the neuronal membrane polarization is an active process that requires cellular ATP as is required for action potential generation, ion concentration restoration, or vesicular recycling (H. Alle, et al., Science 325, 1405-1408 (2009); J. J. Harris, et al., Neuron 75, 762-777 (2012)). With neuronal stimulation, glucose can be directly transported intracellularly through glucose transporters (L. K. Bak et al., J. Neurochem. 109, 87-93 (2009); Díaz-García et al., Cell metabolism 26, 361-374. e364 (2017); Lundgaard et al., Nat Commun 6, 6807 (2015)) unlike neuron-astrocyte lactate shuttle used during resting state glucose metabolism (Yellen, J. Cell Biol. 217, 2235-2246 (2018)). Intra-neuronal glucose can go through the pentose phosphate pathway bypassing glycolytic energy production to reduce oxidative stress (A. Herrero-Mendez et al., Nature Cell Biology 11, 747-752 (2009)). Increased activity and expression of neuronal GLUT transporters during neuronal activity can drive binding of GD2 to the transporter. Based on molecular simulation studies, steric hindrance will not allow GD2 to pass through the GLUT3 transporter (D. S. Dwyer, Proteins: Structure, Function, and Bioinformatics 42, 531-541 (2001)). However, GD2 proximity to neuronal membrane through GLUT receptor interaction can facilitate internalization of GD2-Cy5 through other mechanisms.

GLUT3 transporters are expressed in neuronal dendrites and axons (B. S. McEwen, L. P. Reagan, Eur J Pharmacol 490, 13-24 (2004)). During acute brain injury, synaptic neurotransmission is increased that will necessitate higher synaptic vesicular recycling. In the presence of GD2 in the vicinity, the nanoparticle can be internalized through synaptic vesicular recycling (S. O. Rizzoli, The EMBO journal 33, 788-822 (2014)).

Previous neuron targeting inorganic nanoparticles were limited to surface interactions and not internalization (Dante et al., ACS nano 11, 6630-6640 (2017).). GD2, however, can be internalized into the cytoplasm, to target intracellular organelles and macromolecules. The selectivity of GD2 to target neurons only in brain-injury conditions such as the seizure mice model demonstrated in FIGS. 8A and 8B and not accumulating in neurons in healthy conditions provides much-needed selectivity in neurotherapeutics development. Overall, a dendrimer that intrinsically targets hyperactive neurons and localizes intracellularly was developed in this study.

In summary, the data demonstrates that: Cy5-labeled glucose dendrimer (GD2-Cy5) localizes in hyperexcitable neurons in vitro and in vivo (2) glucose transporters regulate GD2 uptake by neurons, and (3) intranasally delivered glucose dendrimer-valproate conjugate (GD2-VPA) significantly decreases the seizure-severity in a pilocarpine induced mouse model of epilepsy.

Modifications and variations of the present invention will be apparent to those skilled in the art and are intended to come within the scope of the appended claims. All references cited herein are incorporated by reference.

1. A glucose dendrimer, comprising
(a) a central core,
(b) one or more branching units, wherein the branching units are glucose-based branching units, wherein the glucose may be a mono, di or oligosacchardie, optionally with a linker conjugate thereto; and optionally
(c) one or more therapeutic, prophylactic or diagnostic agents,
wherein the one or more branching units are conjugated to the central core, and wherein the surface groups of the dendrimer comprise monosaccharide glucose molecules.

2. The glucose dendrimer of claim 1, wherein the central core is dipentaerythritol, or a hexa-propargylated derivative thereof.

3. The glucose dendrimer of claim 1, wherein the branching unit is conjugated to the central core via a linker selected from a hydrocarbon and an oligoethylene glycol chain.

4. The glucose dendrimer of claim 1, wherein the branching units are β-D-Glucopyranoside tetraethylene glycol azide having the following structure, 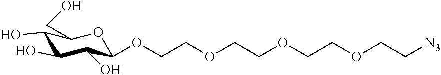
or peracetylated derivatives thereof.

5. The glucose dendrimer of any one of claim 1, wherein the dendrimer is a generation 1, generation 2, generation 3, generation 4, generation 5, or generation 6 dendrimer.

6. The glucose dendrimer of claim 1, wherein the dendrimer is a generation 1 dendrimer having the following structure:

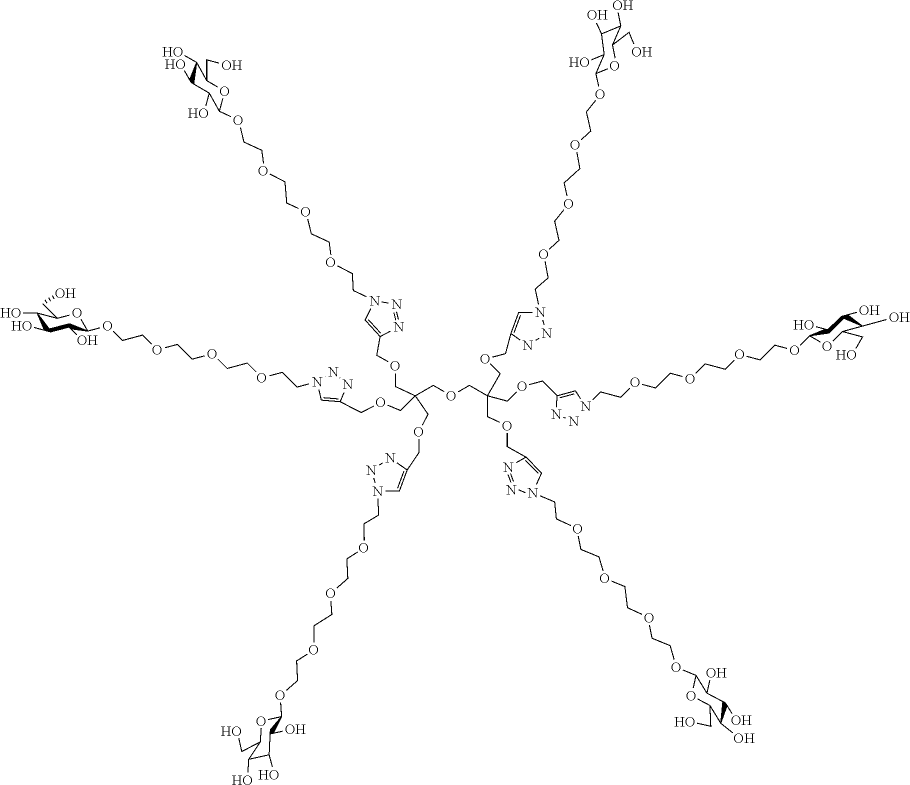

7. The glucose dendrimer of claim 1, wherein the dendrimer is a generation 2 dendrimer having the following structure:

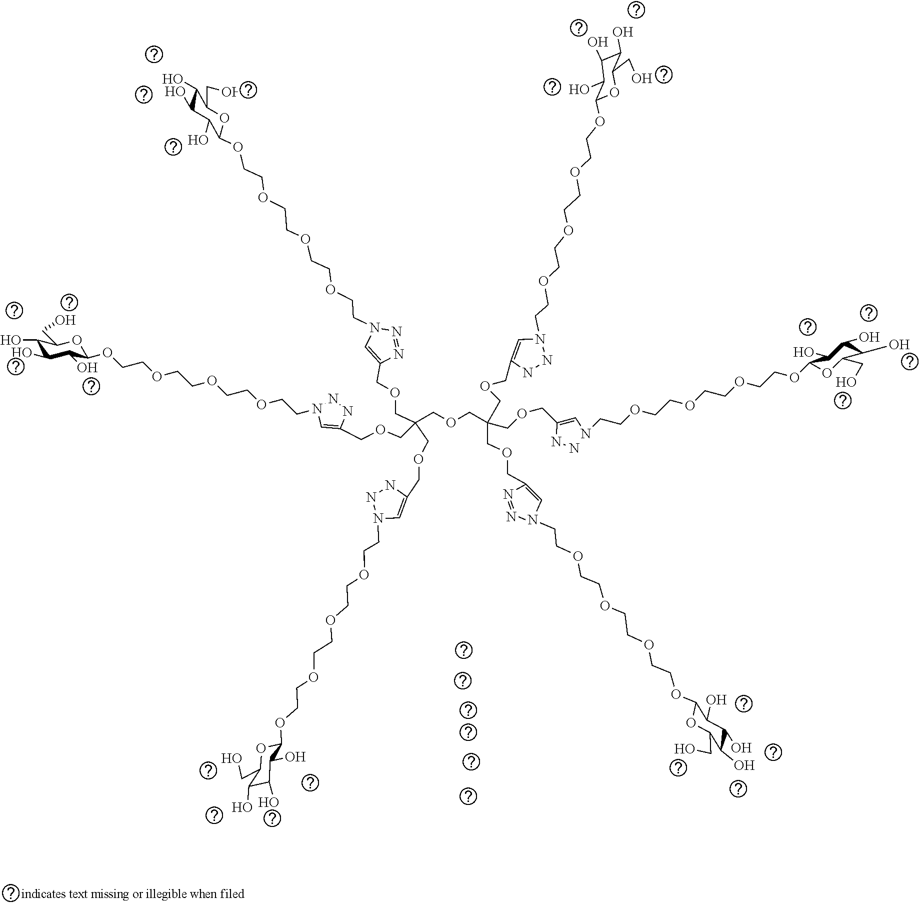

8. The glucose dendrimer of claim 1, wherein the one or more therapeutic, prophylactic or diagnostic agents encapsulated, associated, and/or conjugated in the dendrimer are selected from the group consisting of therapeutic agents, prophylactic agents, and diagnostic agents.

9. The glucose dendrimer of claim 1, the one or more prophylactic, therapeutic, and/or diagnostic agents encapsulated, associated, and/or conjugated in the dendrimer are at a concentration of about 0.01% to about 30%, preferably about 1% to about 20%, more preferably about 5% to about 20% by weight.

10. The glucose dendrimer of claim 1, wherein the dendrimer is conjugated to one or more therapeutic, prophylactic or diagnostic agents selected from the group consisting of a small molecule, an antibody or antigen-binding fragment thereof, a nucleic acid, and a polypeptide.

11. The glucose dendrimer of claim 8, wherein the therapeutic agent is selected from the group consisting of anti-inflammatory agents, antioxidant agents, and immune-modulating agents.

12. The glucose dendrimer of claim 8, wherein the diagnostic agents are selected from the group consisting of fluorescent dyes, near infra-red dyes, SPECT imaging agents, PET imaging agents, and radioisotopes.

13. The glucose dendrimer of claim 1, comprising one or more linkers or coupling agents between the dendrimer and the therapeutic, prophylactic or diagnostic agent.

14. The glucose dendrimer of claim 13, wherein the one or more linkers or coupling agents between the dendrimer and the therapeutic, prophylactic or diagnostic agent, are one or more hydrocarbon or oligoethylene glycol chains.

15. The glucose dendrimer of claim 13, wherein the therapeutic, prophylactic or diagnostic agents are conjugated to the dendrimer via one or more linkages selected from the group consisting of disulfide, ester, ether, thioester, and amide linkages.

16. The dendrimer of claim 1 in combination with a pharmaceutically acceptable carrier or excipient.

17. (canceled)

18. The pharmaceutical formulation of claim 16, wherein the formulation is formulated for enteral or parenteral administration.

19. The pharmaceutical formulation of claim 16, wherein the formulation is formulated for intramuscular, intraperitoneal, intravenous, or subcutaneous injection administration.

20. A method for treating or preventing one or more diseases, conditions, and/or injuries of the eye, the brain and/or the nervous system (CNS) comprising administering to a subject in need thereof the pharmaceutical formulation of claim 16.

21. The method of claim 20, wherein the one or more diseases, conditions, and/or injuries of the eye, the brain and/or the nervous system are diseases, conditions, and injuries associated with neurons and/or activated microglia.

22. The method of claim 20, wherein the one or more diseases, conditions, and/or injuries of the eye are eye diseases associated with retinal ganglion cells selected from the group consisting of glaucoma, diabetic retinopathy, acute retinal ischemia, traumatic optic nerve injury, optic nerve atrophy, and Leber's hereditary optic neuropathy.

23. The method of claim 22, wherein the one or more therapeutic agents encapsulated, associated, and/or conjugated in the dendrimer are selected from the group consisting of ROCK inhibitors, α-2 adrenergic receptor agonists, and caspase inhibitors.

24. The method of claim 20, wherein the one or more diseases, conditions, and/or injuries of the brain and/or the nervous system are neurological and/or neurodegenerative diseases selected from the group consisting of traumatic brain injury, demyelinating diseases, epilepsy, neuralgia, Alzheimer's disease, Parkinson's disease, Huntington's disease, stroke, cerebral palsy, autism, multiple sclerosis, spinal muscular atrophy, neuronal ceroid lipofuscinoses, and neuronopathic Goucher disease.

25. The method of claim 24, wherein the one or more therapeutic agents encapsulated, associated, and/or conjugated in the dendrimer are selected from the group consisting of calpain inhibitors, GPR52 antagonists, NMDA antagonists, mTOR inhibitors, LLRK2 inhibitors, nuclear factor erythroid 2 related factor 2 activators, and SMN-2 promotors.

26. The method of claim 20, wherein the one or more diseases, conditions, and/or injuries of the brain and/or the nervous system are neurological diseases associated with motor neurons.

27. The method of claim 26, wherein the neurological diseases are motor neuron diseases selected from the group consisting of amyotrophic lateral sclerosis, primary lateral sclerosis, progressive bulbar palsy, pseudo bulbar palsy, progressive muscular atrophy, spinal muscular atrophy, Kennedy's disease.

28. The method of claim 26, wherein the neurological disease is spinal muscular atrophy.

29. The method of claim 28, wherein the one or more therapeutic agents encapsulated, associated, and/or conjugated in the dendrimer are HDAC inhibitors or antisense oligonucleotides.

30. The method of claim 29, wherein the antisense oligonucleotides are nusinersen.

31. The method of claim 20, wherein the dendrimer formulation is administered orally, intravenously, intraperitoneally, or intravitreally.

32. The method of claim 20, wherein the amount of therapeutic, prophylactic or diagnostic agent effective to treat or prevent the one or more symptoms is less than the amount of the same therapeutic, prophylactic or diagnostic agent administered in the absence of the glucose dendrimers, or administered as a formulation in combination with dendrimers in the absence of surface glucose molecules.

33-35. (canceled)

36. A method for labeling one or more neurons and/or activated microglia associated with one or more diseases, conditions, and/or injuries of the eye, the brain and/or the nervous system (CNS) comprising administering to the subject the pharmaceutical formulation of claim 33,
wherein the formulation is administered in an amount effective to label one or more cells associated with the one or more diseases, conditions, and/or injuries of the eye, the brain and/or the nervous system (CNS).

37. The method of claim 36, wherein the labeling is used to diagnose or identify the one or more diseases, conditions, and/or injuries of the eye, the brain and/or the nervous system (CNS) in the subject.

38. The method of claim 36, wherein the labeling is used to monitor or guide therapy and/or surgery.

39. The method of claim 36, wherein the dendrimer formulation is administered orally, intravenously, intraperitoneally, or intravitreally.

40. A method of delivering one or more therapeutic, prophylactic or diagnostic agents to one or more neurons in a subject in need thereof comprising administering to a subject in need thereof the pharmaceutical formulation of claim 16.

41. The method of claim 40, wherein the one or more neurons are selected from the group consisting of cerebral cortex neurons, motor neurons, dopaminergic neurons, hypothalamus neurons, thalamus neurons, brain stem neurons, raphe nucleus neurons, Purkinje neurons, retinal ganglion cells, and other neurons in of the central nervous system.

42. The method of claim 40, wherein the amount of the one or more therapeutic, prophylactic or diagnostic agents accumulated within the one or more neurons is at least 5-fold, 10-fold, 20-fold, 30-fold, 40-fold, or 50-fold more than the amount of the same therapeutic, prophylactic or diagnostic agent administered in the absence of the dendrimers, or administered as a formulation in combination with dendrimers in the absence of surface glucose molecules.

43. (canceled)

44. The method of claim 40, wherein the formulation is administered orally, intravenously, intraperitoneally, or intravitreally.

45. A method of making dendrimers with high density surface glucose groups comprising
(a) preparing a hypercore by performing propargylation of a central core, wherein the central core comprises two or more reactive groups for propargylation;
(b) preparing first hyper monomers AB4 from a branching unit having (n+1) reactive groups by conjugating protection groups on n number of reactive groups, and conjugating one azide group onto one of the reactive groups of the branching unit, wherein n is equal or greater than 2;
(c) mixing the hypercore and hyper monomers for copper (I) catalyzed alkyne azide click chemistry to yield a generation 1 dendrimer.

46. The method of claim 45, wherein the method further comprising
(d) propargylation of the generation 1 dendrimer;
(e) preparing second hyper monomers AB4 from a branching unit having (n+1) reactive groups by conjugating protection groups on n number of reactive groups, and conjugating one azide group onto one of the reactive groups of the branching unit, wherein n is equal or greater than 2;
(f) mixing the propargylated generation 1 dendrimer from step (d) and the second hyper monomers from step (e) for copper (I) catalyzed alkyne azide click chemistry to yield a generation 2 dendrimer.

47. The method of claim 45, wherein the central core is dipentaerythritol, or a derivative thereof.

48. The method of claim 45, wherein first and second hyper monomers AB4 are peracetylated β-D-Glucopyranoside tetraethylene glycol azide having the follow structure:

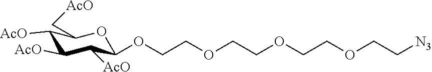

49. The method of claim 45, further comprising a step of deprotecting one or more functional groups of the dendrimer.

50. The method of claim 49, wherein the one or more functional groups of the dendrimer are hydroxyl groups.

51. The method of claim 45, wherein the dendrimer is further complexed and/or conjugated to one or more therapeutic, prophylactic, and/or diagnostic agents.
